(19) United States
(12) Patent Application Publication
(10) Pub. No.: US 2020/0040056 A1
(43) Pub. Date: Feb. 6, 2020

(54) GENOME-EDITED INVARIANT NATURAL KILLER T (INKT) CELLS FOR THE TREATMENT OF HEMATOLOGIC MALIGNANCIES

(71) Applicant: Washington University, St. Louis, MO (US)

(72) Inventors: John DiPersio, St. Louis, MO (US); Matthew Cooper, St. Louis, MO (US); Julie O'Neal, St. Louis, MO (US)

(21) Appl. No.: 16/428,789

(22) Filed: May 31, 2019

Related U.S. Application Data
(60) Provisional application No. 62/678,883, filed on May 31, 2018.

(51) Int. Cl. C07K 14/725; C12N 5/0783; C07K 14/705; A61K 35/17; C12N 15/85; C07K 16/28; A61P 35/00

(52) U.S. Cl. CPC C07K 14/7051; C12N 5/0646; C07K 14/70575; C07K 14/70521; A61K 35/17; C07K 2319/03; C07K 16/2806; C07K 16/2803; A61P 35/00; C07K 16/2878; C07K 2319/02; C12N 15/85

(57) ABSTRACT

Disclosed herein are genome-edited invariant natural killer T (iNKT) cells and methods of immunotherapy using them. In particular, the disclosure relates to engineered chimeric antigen receptor (CAR)-bearing INKT cells (CAR-iNKTs) and methods of using the same for the treatment of cancer.

This application claims the benefit of priority of U.S. Provisional Application No. 62/678,883, filed May 31, 2018, the disclosure of which is hereby incorporated by reference as if written herein in its entirety.

### SEQUENCE LISTING

The instant application contains a Sequence Listing which has been submitted electronically in ASCII format and is hereby incorporated by reference in its entirety. Said ASCII copy, created on Aug. 14, 2019, is named WGN0002-401-PC.txt and is 301,002 bytes in size.

Disclosed herein are genome-edited invariant natural killer T (iNKT) cells and methods of immunotherapy using them. In particular, the disclosure relates to engineered chimeric antigen receptor (CAR)-bearing INKT cells (CAR-iNKTs) and methods of using the same for the treatment of cancer.

Chimeric antigen receptor T cell (CAR-T) immunotherapy is increasingly well known. T cells are genetically modified to express chimeric antigen receptors (CARs), which are fusion proteins comprised of an antigen recognition moiety and T cell activation domains. The CARs are designed to recognize antigens that are overexpressed on cancer cells. CAR-Ts demonstrate exceptional clinical efficacy against B cell malignancies, and two therapies, Kymriah™ (tisagenlecleucel, Novartis) and Yescarta™ (axicabtagene ciiloleucel, Kite/Gilead), were recently approved by the FDA. However, broad applicability of CAR-T therapy has been limited in two ways. First, the development of CAR-T cell therapy against T cell malignancies has proven problematic, in part due to the shared expression of target antigens between malignant T cells and effector T cells, because expression of target antigens on CAR-T cells may induce fratricide of CAR-T cells and loss of efficacy. Second, the use of T-cells other than an individual patient's own (allogenic) in CAR-T therapy may lead to allogenic reactivity including graft-versus-host disease.

Invariant natural killer T cells, also called iNKT cells or type-I NKT cells, represent a distinct lymphocyte population, characterized by expression of an invariant T cell receptor α-chain and certain TCR β-chains (Vα24-Jα18 combined with Vβ11). iNKT TCR-mediated responses are restricted by CD1d, a member of the non-polymorphic CD1 antigen presenting protein family, which promotes the presentation of endogenous and pathogen-derived lipid antigens to the TCR. The prototypical ligand for invariant receptor is α-Galactosylceramide (αGalCer). Upon binding of the invariant TCR to CD1d-αGalCer, iNKT will expand. The CD1d gene is monomorphic and expressed by only a few cell types, limiting the potential toxicity of NKT cells in the autologous or allogeneic settings.

### BRIEF DESCRIPTION OF THE DRAWINGS

FIG. 1 shows a method of producing genome-edited CAR-iNKT cells targeting one or more targets, for example CD7.

FIG. 2 shows a method of producing genome-edited tandem CAR-iNKT cells targeting two targets, here, CD7 and CD2.

FIG. 3 shows a method of producing genome-edited CAR-iNKT cells targeting CD7.

FIG. 4 shows a method of producing genome-edited CAR-iNKT cells targeting another target, designated “A.”

FIG. 5 shows a method of producing genome-edited tandem CAR-iNKT cells targeting CD7 and CD2.

FIG. 6 shows a method of producing genome-edited dual CAR-iNKT cells targeting two other targets, designated “A” and “B.”

FIG. 7 shows a flow diagram of a method of treatment of cancer (for example, T-cell malignancies) by preparing and infusing gene-edited iNKT cells.

FIG. 8. CAR-iNKT effectivity kill tumor cells in vitro and in vivo. FIG. 8A. shows transduction efficiency of iNKT with CAR19 and CAR-BCMA. FIG. 8B shows CAR19 and CAR-BCMA specifically kill antigen positive target cells (Ramos) in 4 hr Cr release assay. FIG. 8C shows iNKT-BCMA effectively kill MM1.s cells in vivo. FIG. 8D shows iNKT-BCMA prolongs survival of mice in xenogeneic MM1.s mouse model of multiple myeloma.

FIG. 9 shows CAR2-iNKT cells effectively killing CD2⁺ T-ALL and CTCL cell lines in vitro.

### DETAILED DESCRIPTION

The following disclosure will detail embodiments, alternatives, and uses of engineered iNKT cells such as genome-edited iNKT cells, CAR-iNKT cells, dual-CAR iNKT cells, and tandem-CAR iNKT cells, as well as the uses of such cells in, for example immunotherapy and adoptive cell transfer for the treatment of diseases. Accordingly, provided herein are the following embodiments.

### Embodiment 1

A genome-edited iNKT cell.

### Embodiment 2

A population of genome-edited iNKT cells as recited in embodiment 1, wherein the genome-edited iNKT cells are from multiple donors that can be maintained or expanded for at least three weeks without being frozen.

### Embodiment 3

The iNKT cell as recited in embodiment 1, wherein the iNKT cell comprises at least one chimeric antigen receptor (CAR) targeting one or more antigens, and wherein the iNKT cell is deficient in an antigen to which the CAR specifically binds

### Embodiment 4

The iNKT cell as recited in any of embodiments 1 to 3, wherein the chimeric antigen receptor specifically binds at least one antigen expressed on a malignant T cell.

### Embodiment 5

The iNKT cell, as recited in any of embodiments 1 to 4, wherein the antigen is selected from CD2, CD3ε, CD4, CD5, CD7, TRAC, and TCRβ.

### Embodiment 6

The iNKT cell, as recited in any of embodiments 1 to 5, wherein the chimeric antigen receptor specifically binds at least one antigen expressed on a malignant plasma cell.

### Embodiment 7

The iNKT cell, as recited in any of embodiments 1 to 6, wherein the antigen is selected from BCMA, CS1, CD38, and CD19.

### Embodiment 8

The iNKT cell, as recited in any of embodiments 1 to 7, wherein the chimeric antigen receptor expresses the extracellular portion of the APRIL protein, the ligand for BCMA and TACI, effectively co-targeting both BCMA and TACI.

### Embodiment 9

The iNKT cell, as recited in any of embodiments 1 to 8, wherein the CAR-T cell further comprises a suicide gene.

### Embodiment 10

The iNKT cell, as recited in any of embodiments 1 to 9, wherein endogenous T cell receptor mediated signaling is negligible in the iNKT cell.

### Embodiment 11

The iNKT cell, as recited in any of embodiments 1 to 10, wherein the iNKT cells do not induce alloreactivity or graft-versus-host disease.

### Embodiment 12

The iNKT cell as recited in any of embodiments 1 to 11, wherein the iNKT cells do not induce fratricide.

### Embodiment 13

The iNKT cell as recited in any of Embodiments 1-12, wherein the chimeric antigen receptor(s) specifically binds at least one antigen expressed on a malignant B cell.

### Embodiment 14

The iNKT cell as recited in Embodiment 13, wherein the antigen expressed on a malignant B cell is chosen from CD19, CD20, CD21, CD22, CD23, CD24, CD25, CD27, CD38, and CD45.

### Embodiment 15

The iNKT cell as recited in Embodiment 14, wherein the antigen expressed on a malignant B cell is chosen from CD19 and CD20.

### Embodiment 16

The iNKT cell, as recited in any of embodiments 1 to 15, wherein the iNKT cell is a dual iNKT-CAR cell.

### Embodiment 17

A dual iNKT-CAR cell as recited in embodiment 16, wherein the dual iNKT-CAR cell comprises two or more CARs each targeting different T cell antigens.

### Embodiment 18

The dual iNKT-CAR cell as recited in embodiment 16 and 17, wherein the different T-cell antigens are chosen from CD2×CD3ε, CD2×CD4, CD2×CD5, CD2×CD7, CD3εxCD4, CD3εxCD5, CD3εxCD7, CD4×CD5, CD4×CD7, CD5×CD7, TRAC×CD2, TRAC×CD3ε, TRAC×CD4, TRAC×CD5, TRAC×CD7, TCRβ×CD2, TCRβ×CD3ε, TCRβ×CD4, TCRβ×CD5, TCRβ×CD7, BCMA×CS1, BCMA×CD19, BCMA×CD38, CS1×CD19, CS1×CD38, CD19×CD38, APRIL×CS1, APRIL×BCMA, APRIL×CD19, and APRIL×CD38

### Embodiment 19

The dual iNKT-CAR cell as recited in embodiments 16 to 18, wherein each of the V_{H} and V_{L} chains is derived from an scFv that recognizes a different antigen is chosen from CD5, CD7, CD2, CD4, and CD3.

### Embodiment 20

The dual iNKT-CAR cell as recited in embodiments 16 to 19, wherein each of the V_{H} and V_{L} chains is different and displays at least 95% sequence identity to an amino acid sequence chosen from SEQ ID NO:12 to SEQ ID NO:31.

### Embodiment 21

The dual iNKT-CAR cell as recited in embodiments 16 and 20, wherein each of the V_{H} and V_{L} chains is different and displays at least 98% sequence identity to an amino acid sequence chosen from SEQ ID NO:12 to SEQ ID NO:31.

### Embodiment 22

The dual iNKT-CAR cell as recited in embodiments 16 and 21, wherein each of the V_{H} and V_{L} chains is different and is a sequence chosen from SEQ ID NO:12 to SEQ ID NO:31.

### Embodiment 23

The dual iNKT-CAR cell as recited in embodiments 16 and 22, comprising at least one costimulatory domain chosen from CD28 and 4-1BB.

### Embodiment 24

The dual iNKT-CAR cell as recited in embodiments 16 and 23, wherein the costimulatory domain is CD28.

### Embodiment 25

The dual iNKT-CAR cell as recited in embodiments 16 and 24, comprising a CD3ζ signaling domain.

### Embodiment 26

The dual iNKT-CAR cell as recited in embodiments 16 and 25, wherein the each of the V_{H} and V_{L} chains is derived from an scFv recognizing CD2 or an scFv recognizing CD3.

### Embodiment 27

The tandem iNKT-CAR cell as recited in embodiments 1 to 15, wherein the tandem iNKT-CAR cell comprises one CAR targeting two or more T cell antigens.

### Embodiment 28

The tandem iNKT-CAR cell as recited in embodiment 27, wherein the antigen pair is chosen from CD2×CD3ε, CD2×CD4, CD2×CD5, CD2×CD7, CD3εxCD4, CD3εxCD5, CD3εxCD7, CD4×CD5, CD4×CD7, CD5×CD7, TRAC×CD2, TRAC×CD3ε, TRAC×CD4, TRAC×CD5, TRAC×CD7, TCRβ×CD2, TCRβ×CD3ε, TCRβ×CD4, TCRβ×CD5, TCRβ×CD7, BCMA×CS1, BCMA×CD19, BCMA×CD38, CS1×CD19, CS1×CD38, CD19×CD38, APRIL×CS1, APRIL×BCMA, APRIL×CD19, and APRIL×CD38.

### Embodiment 29

The tandem iNKT-CAR cell as recited in embodiments 27 and 28, wherein the linear tCAR construct comprises a first heavy (V_{H}) chain variable fragment and a first light (V_{L}) chain variable fragment, designated V_{H}1 and V_{L}1, joined by a (GGGGS)₂₋₆ (SEQ ID NO:447) linker to a second light (V_{L}) chain variable fragment and a first heavy (V_{H}) chain variable fragment, designated V_{L}2 and V_{H}2.

### Embodiment 30

The tandem iNKT-CAR cell as recited in embodiments 27 to 29, wherein the linear tCAR construct comprises a first heavy (V_{H}) chain variable fragment and a first light (V_{L}) chain variable fragment, designated V_{H}2 and V_{L}2, joined by a (GGGGS)₂₋₆ (SEQ ID NO:447) linker to a second light (V_{L}) chain variable fragment and a first heavy (V_{H}) chain variable fragment, designated V_{H}1 and V_{L}1.

### Embodiment 31

The tandem iNKT-CAR cell as recited in embodiments 27 and 30, wherein the linear tCAR construct comprises a first light (V_{L}) chain variable fragment and a first heavy (V_{H}) chain variable fragment, designated V_{L}1 and V_{H}1, joined by a (GGGGS)₂₋₆ (SEQ ID NO:447) linker to a second heavy (V_{H}) chain variable fragment and a first light (V_{L}) chain variable fragment, designated V_{H}2 and V_{L}2.

### Embodiment 32

The tandem iNKT-CAR cell as recited in embodiments 27 and 31, wherein the linear tCAR construct comprises a first light (V_{L}) chain variable fragment and a first heavy (V_{H}) chain variable fragment, designated V_{L}2 and V_{H}2, joined by a (GGGGS)₂₋₆ (SEQ ID NO:447) linker to a second heavy (V_{H}) chain variable fragment and a first light (V_{L}) chain variable fragment, designated V_{H}1 and V_{L}1.

### Embodiment 33

The tandem iNKT-CAR cell as recited in embodiments 27 and 32, wherein the linear tCAR construct comprises a structure chosen from 6-I to 6-XXXII.

### Embodiment 34

The tandem iNKT-CAR cell as recited in embodiments 27 and 33, wherein the CAR construct is a hairpin tCAR construct.

### Embodiment 35

The tandem iNKT-CAR cell as recited in embodiments 27 and 34, wherein the hairpin tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₂₋₆ (SEQ ID NO:447) linker to a first light (V_{L}) chain variable fragment derived from the second scFv, and a second light (V_{L}) chain variable fragment derived from the first scFv, designated V_{L}2 and V₁2.

### Embodiment 36

The tandem iNKT-CAR cell as recited in embodiments 27 and 35, wherein the hairpin tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₂₋₆ (SEQ ID NO:447) linker to a first light (V_{L}) chain variable fragment derived from the first scFv, and a second light (V_{L}) chain variable fragment derived from the second scFv, designated V_{L}1 and V_{L}2.

### Embodiment 37

The tandem iNKT-CAR cell as recited in embodiments 27 and 36, wherein the hairpin tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₂₋₆ (SEQ ID NO:447) linker to a first heavy (V_{H}) chain variable fragment derived from the first scFv, and a second heavy (V_{L}) chain variable fragment derived from the second scFv, designated V_{H}2 and V_{H}1.

### Embodiment 38

The tandem iNKT-CAR cell as recited in embodiments 27 and 37, wherein the hairpin tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₂₋₆ (SEQ ID NO:447) linker to a first heavy (V_{H}) chain variable fragment derived from the first scFv, and a second light heavy (V_{H}) variable fragment derived from the second scFv, designated V_{H}1 and V_{H}2.

### Embodiment 39

The tandem iNKT-CAR cell as recited in embodiments 27 and 38, wherein the hairpin tCAR construct comprises a structure chosen from 8-I to 8-XXXII.

### Embodiment 40

The tandem iNKT-CAR cell as recited in embodiments 27 and 39, wherein the CAR construct is a hairpin DSB tCAR construct with a (Cys=Cys) Double-Stranded Bond (DSB) in the linker.

### Embodiment 41

The tandem iNKT-CAR cell as recited in embodiments 27 and 40, wherein the hairpin DSB tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first light (V_{L}) chain variable fragment derived from the second scFv, and a second light (V_{L}) chain variable fragment derived from the first scFv, designated V_{L}2 and V₁2.

### Embodiment 42

The tandem iNKT-CAR cell as recited in embodiments 27 and 41, wherein the hairpin DSB tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first light (V_{L}) chain variable fragment derived from the first scFv, and a second light (V_{L}) chain variable fragment derived from the second scFv, designated V_{L}1 and V_{L}2.

### Embodiment 43

The tandem iNKT-CAR cell as recited in embodiments 27 and 42, wherein the hairpin DSB tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first heavy (V_{H}) chain variable fragment derived from the first scFv, and a second heavy (V_{L}) chain variable fragment derived from the second scFv, designated V_{H}2 and V_{H}1.

### Embodiment 44

The tandem iNKT-CAR cell as recited in embodiments 27 and 43, wherein the hairpin DSB tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first heavy (V_{H}) chain variable fragment derived from the first scFv, and a second light heavy (V_{H}) variable fragment derived from the second scFv, designated V_{H}1 and V_{H}2.

### Embodiment 45

The tandem iNKT-CAR cell as recited in embodiments 27 and 44, wherein the hairpin DSB tCAR construct comprises a structure chosen from 10-I to 10-XXXII.

### Embodiment 46

The tandem iNKT-CAR cell as recited in embodiments 27 and 45, wherein each of the V_{H} and V_{L} chains is derived from an scFv that recognizes a different antigen chosen from CD5, CD7, CD2, CD4, and CD3.

### Embodiment 47

The tandem iNKT-CAR cell as recited in embodiments 27 and 46, wherein each of the V_{H} and V_{L} chains is different and displays at least 95% sequence identity to an amino acid sequence chosen from SEQ ID NO:12 to SEQ ID NO:31.

### Embodiment 48

The tandem iNKT-CAR cell as recited in embodiments 27 and 47, wherein each of the V_{H} and V_{L} chains is different and displays at least 98% sequence identity to an amino acid sequence chosen from SEQ ID NO:12 to SEQ ID NO:31.

### Embodiment 49

The tandem iNKT-CAR cell as recited in embodiments 27 and 48, wherein each of the V_{H} and V_{L} chains is different and is a sequence chosen from SEQ ID NO:12 to SEQ ID NO:31.

### Embodiment 50

The tandem iNKT-CAR cell as recited in embodiments 27 and 49, comprising at least one costimulatory domain chosen from CD28 and 4-1BB.

### Embodiment 51

The tandem iNKT-CAR cell as recited in embodiments 27 and 50, wherein the costimulatory domain is CD28.

### Embodiment 52

The tandem iNKT-CAR cell as recited in embodiments 27 and 51, comprising a CD3_{ζ} signaling domain.

### Embodiment 53

The tandem iNKT-CAR cell as recited in embodiments 27 and 52, wherein the each of the V_{H} and V_{L} chains is derived from an scFv recognizing CD2 or an scFv recognizing CD3.

### Embodiment 54

The tandem iNKT-CAR cell as recited in embodiments 27 and 53, wherein the tCAR construct is chosen from Clone 5, Clone 6, Clone 7, Clone 8, Clone 13, Clone 14, Clone 15, and Clone 16.

### Embodiment 55

The tandem iNKT-CAR cell as recited in embodiments 27 and 54, wherein the tCAR construct displays at least 95% sequence identity to an amino acid sequence chosen from SEQ ID NO:41 to SEQ ID NO:46.

### Embodiment 55

A therapeutic composition comprising the iNKT cells as recited in any of embodiments 1 to 54, and at least one therapeutically acceptable carrier and/or adjuvant.

### Embodiment 56

A therapeutic composition comprising the iNKT cells as recited in any of embodiments 1 to 55, wherein the composition comprises at least one adjuvant chosen from IL-7, IL-15, 11-2, or an analogue of any of the foregoing.

### Embodiment 57

A therapeutic composition comprising the iNKT cells as recited in any of embodiments 1 to 56, wherein the composition comprises IL-2.

### Embodiment 58

A therapeutic composition comprising the iNKT cells as recited in any of embodiments 1 to 57, wherein the composition comprises a combination of any two or more of IL-7, IL-15, 11-2, or an analogue of any of the foregoing.

### Embodiment 59

A therapeutic composition comprising the iNKT cells as recited in any of embodiments 1 to 58, wherein the composition comprises IL-7, IL-15, 11-2.

### Embodiment 60

A method of treatment of a hematologic malignancy in a patient comprising administering genome-edited iNKT cell, population of genome-edited iNKT cells, dual iNKT-CAR cell, or tandem iNKT-CAR cell as recited in any of embodiments 1 to 59, or the therapeutic composition as recited in any of claims 55-59 to a patient in need thereof.

### Embodiment 61

The method as recited in embodiment 60, wherein the hematologic malignancy is a T-cell malignancy.

### Embodiment 62

The method as recited in embodiment 61, wherein the T cell malignancy is T-cell acute lymphoblastic leukemia (T-ALL).

### Embodiment 63

The method as recited in embodiment 61, wherein the T cell malignancy is non-Hodgkins lymphoma.

### Embodiment 64

The method as recited in embodiment 60, wherein the hematologic malignancy is multiple myeloma.

### Embodiment 65

A method of making a gene-edited iNKT cell comprising the steps of:
a) activating isolated and purified iNKT cells;
b) deleting or suppressing expression of a cell surface protein in the iNKT cell; and
c) optionally, transducing the iNKT cell with a chimeric antigen receptor that recognizes one or more antigen or cell surface protein targets.

### Embodiment 66

The method as recited in embodiment 65, which includes the step of transducing the iNKT cell with a chimeric antigen receptor that recognizes one or more antigen or cell surface protein targets.

### Embodiment 67

The method as recited in embodiment 66, wherein the antigen that is the target of the CAR is deleted from the cell.

### Embodiment 68

A method of making a population of genome-edited iNKT cells from multiple donors comprising the steps of:
a) activating isolated and purified iNKT cells from each donor;
b) deleting or suppressing expression of a cell surface protein in the iNKT cell;
c) optionally, transducing the iNKT cell with a chimeric antigen receptor that recognizes one or more antigen or cell surface protein targets;
d) expanding the population of genome-edited iNKT cells;
e) pooling the genome-edited iNKT cells.

### Embodiment 69

A method of making a CAR-T cell as recited in any embodiment above or herein, using Cas9-CRISPR and a gRNA chosen from those disclosed herein.

### Embodiment 70

A method of making a CAR-T cell as recited in any embodiment above or herein, using Cas9-CRISPR and a gRNA chosen from those disclosed Table 12 and Tables 14-26.

### Embodiment 71

A method of making a CAR-T cell as recited in any embodiment above or herein, using Cas9-CRISPR and a gRNA chosen from those disclosed in Table 12 and those in boldface in Tables 14-26.

### Embodiment 72

A method of making a CAR-T cell as recited in any embodiment above or herein, using Cas9-CRISPR and a gRNA chosen from those disclosed in Tables 12.

Disclosed herein are genome-edited invariant natural killer T (iNKT) cells.

Also provided is a population of genome-edited iNKT cells from multiple donors that can be maintained or expanded for at least three weeks without being frozen.

Also provided is an iNKT cell, which comprises at least one chimeric antigen receptor (CAR) targeting one or more antigens, and which is deficient in an antigen to which the CAR specifically binds.

In certain embodiments, the chimeric antigen receptor specifically binds at least one antigen expressed on a malignant T cell.

In certain embodiments, the antigen is selected from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

In certain embodiments, the chimeric antigen receptor specifically binds at least one antigen expressed on a malignant plasma cell.

In certain embodiments, the antigen is selected from BCMA, CS1, CD38, and CD19.

In certain embodiments, the chimeric antigen receptor(s) specifically binds at least one antigen expressed on a malignant B cell.

In certain embodiments, the antigen expressed on a malignant B cell is chosen from CD19, CD20, CD21, CD22, CD23, CD24, CD25, CD27, CD38, and CD45.

In certain embodiments, the antigen expressed on a malignant B cell is chosen from CD19 and CD20.

In certain embodiments, the chimeric antigen receptor expresses the extracellular portion of the APRIL protein, the ligand for BCMA and TACI, effectively co-targeting both BCMA and TACI.

In certain embodiments, the iNKT cell further comprises a suicide gene.

In certain embodiments, endogenous T cell receptor mediated signaling is blocked in the iNKT cell.

In certain embodiments, the iNKT cells do not induce alloreactivity or graft-versus-host disease.

In certain embodiments, the iNKT cells do not induce fratricide.

Also provided is a dual or tandem iNKT-CAR cell.

Also provided is a therapeutic composition comprising the population of iNKT cells as disclosed herein, and at least one therapeutically acceptable carrier and/or adjuvant.

In certain embodiments, the composition comprises at least one adjuvant chosen from IL-7, IL-15, IL-2, or an analogue of any of the foregoing.

In certain embodiments, the composition comprises IL-2.

In certain embodiments, the composition comprises a combination of any two or more of IL-7, IL-15, IL-2, or an analogue of any of the foregoing.

In certain embodiments, the composition comprises IL-7, IL-15, and IL-2.

Also provided is a method of treatment of a hematologic malignancy in a patient comprising administering genome-edited iNKT cell, population of genome-edited iNKT cells, dual iNKT-CAR cell, or tandem iNKT-CAR cell as disclosed herein, or the therapeutic composition as disclosed herein to a patient in need thereof.

In certain embodiments, the hematologic malignancy is a T-cell malignancy.

In certain embodiments, the T cell malignancy is T-cell acute lymphoblastic leukemia (T-ALL).

In certain embodiments, the T cell malignancy is non-Hodgkins lymphoma.

In certain embodiments, the hematologic malignancy is multiple myeloma.

Also provided is a method of making a gene-edited iNKT cell comprising the steps of:
d) activating isolated and purified iNKT cells;
e) deleting or suppressing expression of a cell surface protein in the iNKT cell; and
f) optionally, transducing the iNKT cell with a chimeric antigen receptor that recognizes one or more antigen or cell surface protein targets.

In certain embodiments, the method includes the step of transducing the iNKT cell with a chimeric antigen receptor that recognizes one or more antigen or cell surface protein targets.

In certain embodiments, the antigen that is the target of the CAR is deleted from the cell.

Also provided is a method of making a population of genome-edited iNKT cells from multiple donors comprising the steps of:
f) activating isolated and purified iNKT cells from each donor;
g) deleting or suppressing expression of a cell surface protein in the iNKT cell;
h) optionally, transducing the iNKT cell with a chimeric antigen receptor that recognizes one or more antigen or cell surface protein targets;
i) expanding the population of genome-edited iNKT cells; and
j) pooling the genome-edited iNKT cells.

Other embodiments are disclosed below.

Genome-Edited iNKT Cells and iNKT-CARs

Fratricide Resistance.

iNKT cells disclosed herein may be deficient in an antigen to which the chimeric antigen receptor specifically binds and are therefore fratricide-resistant. In some embodiments, the antigen of the iNKT cell is modified such that the chimeric antigen receptor no longer specifically binds the modified antigen. For example, the epitope of the antigen recognized by the chimeric antigen receptor may be modified by one or more amino acid changes (e.g., substitutions or deletions) or the epitope may be deleted from the antigen. In other embodiments, expression of the antigen is reduced in the iNKT cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T iNKT cell is modified such that the antigen is not expressed, e.g., by deletion or disruption of the gene encoding the antigen. In each of the above embodiments, the iNKT cell may be deficient in one or preferably all the antigens to which the chimeric antigen receptor specifically binds. Methods for genetically modifying an iNKT cell to be deficient in an antigen are well known in art, and non-limiting examples are provided above. In an exemplary embodiment, CRISPR/cas9 gene editing can be used to modify an iNKT cell to be deficient in an antigen, for example as described below. Alternatively, TALENs may be used to edit genes.

In an variation of the method above, a construct encoding one or more protein expression blocker (PEBL) may be transduced into the cell, either as the editing step or part of the editing step, or as part of CAR transduction. For example, an construct encoding an antibody-derived single-chain variable fragment specific for CD3ε may be transduced, e.g. by a lentiviral vector. Once expressed, the PEBL colocalizes intracellularly with CD3ε, blocking surface CD3 and TCRαβ expression. Accordingly, PEBL blockade of surface CD3/TCRαβ expression is an alternative method of preparing allogeneic CAR-T cells. Furthermore, PEBL and CAR expression can be combined in a single construct. Either of these methods may be achieved using the methods disclosed herein, and PEBLs may be produced for blockade of any of the targets of gene suppression disclosed herein.

The methods described above may be adapted to insert a CAR into a locus for a gene encoding an antigen, cell surface protein, or secretable protein, such as a cytokine. In this way, editing of the genome is effected by transfection of CAR. Thereafter, cells may be activated as described herein, removing separate genome editing step in certain embodiments. Ideally, such a step should be performed while cells are actively dividing. Such methods are also expected to result in robust expansion of engineered cells.

In certain circumstances, an iNKT cell may be selected for deficiency in the antigen to which the chimeric antigen receptor specifically binds. Certain iNKT cells will produce and display less of a given surface protein; instead if deleting or non-functionalizing the antigen that will be the target of the iNKT-CAR, the iNKT cell can be selected for deficiency in the antigen, and the population of antigen-deficient cells expanded for transduction of the CAR. Such a cell would also be fratricide-resistant.

CAR Antigens.

Suitable antigens to be genome-edited in the iNKT cells disclosed herein, and to be recognized by the CARs of iNKT-CARs disclosed herein, include antigens specific to hematologic malignancies. These can include T cell-specific antigens and/or antigens that are not specific to T cells. The antigen may be specifically bound by the chimeric antigen receptor of an iNKT-CARs cell, and the antigen for which the iNKT-CARs cell is deficient, is an antigen expressed on a malignant T cell, preferably an antigen that is overexpressed on malignant T cell (i.e., a T cell derived from a T-cell malignancy) in comparison to a nonmalignant T cell. Examples of such antigens include BCMA, CS1, CD38, CD138, CD19, CD33, CD123, CD371, CD117, CD135, Tim-3, CD5, CD7, CD2, CD4, CD3ε, CD79A, CD79B, APRIL, CD56, and CD1a, TRAC, and TCRβ.

T-cell malignancies comprise malignancies derived from T-cell precursors, mature T cells, or natural killer cells. Examples of T-cell malignancies include T-cell acute lymphoblastic leukemia/lymphoma (T-ALL), T-cell large granular lymphocyte (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCLs), including but not limited to angioimmunoblastic T-cell lymphoma (AITL), ALK-positive anaplastic large cell lymphoma, and ALK-negative anaplastic large cell lymphoma.

Suitable CAR antigens can also include antigens found on the surface of a multiple myeloma cell, i.e., a malignant plasma cell, such as BCMA, CS1, CD38, and CD19. Alternatively, the CAR may be designed to express the extracellular portion of the APRIL protein, the ligand for BCMA and TACI, effectively co-targeting both BCMA and TACI for the treatment of multiple myeloma.

Additional examples of suitable antigens to be genome-edited in the iNKT cells disclosed herein, and to be recognized by the CARs of iNKT-CARs disclosed herein, are given below in Tables 4, 5, 11. These include BCMA, CS1, CD38, CD138, CD19, CD33, CD123, CD371, CD117, CD135, Tim-3, CD5, CD7, CD2, CD4, CD3ε, CD79A, CD79B, APRIL, CD56, and CD1a, CD2, CD3ε, CD4, CD5, CD7, TRAC, and TCRβ.

Suicide Genes.

Alternatively, or in addition, genome-edited iNKT cells may further comprise one or more suicide genes. As used herein, “suicide gene” refers to a nucleic acid sequence introduced into an iNKT cell by standard methods known in the art that, when activated, results in the death of the iNKT cell. Suicide genes may facilitate effective tracking and elimination of the iNKT cells in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

Methods of CAR and iNKT-CAR Construction

A “chimeric antigen receptor (CAR),” as used herein and generally used in the art, refers to a recombinant fusion protein that has an antigen-specific extracellular domain (antigen recognition domain) coupled to an intracellular domain (signaling domain) that directs the cell to perform a specialized function upon binding of an antigen to the extracellular domain. Chimeric antigen receptors are distinguished from other antigen binding agents by their ability to both bind MHC-independent antigen and transduce activation signals via their intracellular domain.

Methods for CAR design, delivery and expression, and the manufacturing of clinical-grade iNKT cell populations are known in the art. See, for example, Lee et al., Clin. Cancer Res., 2012, 18(10): 2780-90. An engineered chimeric antigen receptor polynucleotide that encodes for a CAR comprises: a signal peptide, an extracellular ligand-binding domain, i.e., an antigen-recognition domain, a transmembrane domain, and a signaling transducing domain.

The extracellular ligand-binding domain of a chimeric antigen receptor recognizes and specifically binds an antigen, typically a surface-expressed antigen of a malignancy. An “antigen-specific extracellular domain” (or, equivalently, “antigen-binding domain”) specifically binds an antigen when, for example, it binds the antigen with an affinity constant or affinity of interaction (KD) between about 0.1 pM to about 10 μM, preferably about 0.1 pM to about 1 μM, more preferably about 0.1 pM to about 100 nM. Methods for determining the affinity of interaction are known in the art. An extracellular ligand-binding domain suitable for use in a CAR of the present disclosure may be any antigen-binding polypeptide, a wide variety of which are known in the art. In some instances, the antigen-binding domain is a single chain Fv (scFv). Other antibody-based recognition domains (cAb V_{HH} (camelid antibody variable domains) and humanized versions thereof, IgNAR V_{H} (shark antibody variable domains) and humanized versions thereof, sdAb V_{H} (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

A chimeric antigen receptor of the present disclosure also comprises an “intracellular domain” that provides an intracellular signal to the iNKT cell upon antigen binding to the antigen-specific extracellular domain. The intracellular signaling domain of a chimeric antigen receptor of the present disclosure is responsible for activation of at least one of the effector functions of the iNKT cell in which the chimeric receptor is expressed. The term “effector function” refers to a specialized function of a differentiated cell, such as an iNKT cell. An effector function of an iNKT cell, for example, may be NK transactivation, T cell activation and differentiation, B cell activation, dendritic cell activation and cross-presentation activity, and macrophage activation. Thus, the term “intracellular domain” refers to the portion of a CAR that transduces the effector function signal upon binding of an antigen to the extracellular domain and directs the iNKT cell to perform a specialized function. Non-limiting examples of suitable intracellular domains include the zeta chain of the T-cell receptor or any of its homologs (e.g., eta, delta, gamma, or epsilon) and combinations of signaling molecules, such as CD3ζ and CD28, CD27, 4-1 BB, DAP-1 0, OX40, and combinations thereof, as well as other similar molecules and fragments. Intracellular signaling portions of other members of the families of activating proteins may be used, such as FcγRIII and FcεRI. While usually the entire intracellular domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular domain is thus meant to include any truncated portion of the intracellular domain sufficient to transduce the effector function signal.

Typically, the antigen-specific extracellular domain is linked to the intracellular domain of the chimeric antigen receptor by a “transmembrane domain.” A transmembrane domain traverses the cell membrane, anchors the CAR to the T cell surface, and connects the extracellular domain to the intracellular signaling domain, thus impacting expression of the CAR on the T cell surface. Chimeric antigen receptors may also further comprise one or more costimulatory domain and/or one or more spacer. A “costimulatory domain” is derived from the intracellular signaling domains of costimulatory proteins that enhance cytokine production, proliferation, cytotoxicity, and/or persistence in vivo. A “peptide hinge” connects the antigen-specific extracellular domain to the transmembrane domain. The transmembrane domain is fused to the costimulatory domain, optionally a costimulatory domain is fused to a second costimulatory domain, and the costimulatory domain is fused to a signaling domain, not limited to CD3ζ. For example, inclusion of a spacer domain between the antigen-specific extracellular domain and the transmembrane domain, and between multiple scFvs in the case of tandem CAR, may affect flexibility of the antigen-binding domain(s) and thereby CAR function. Suitable transmembrane domains, costimulatory domains, and spacers are known in the art.

TABLE 1
Amino acid sequences of different CAR components.

| | SEQ ID | |
|---|---|---|
| Functional domains | NO: | Amino acid sequence |
| CD8α signal peptide | SEQ ID | MALPVTALLLPLALLLHAARP |
| | NO: 1 | |
| CD8α hinge | SEQ ID | TTTPAPRPPTPAPTIASQPLSLRPEACRPAAGGA |
| | NO: 2 | VHTRGLDFACD |
| CD28 Transmembrane | SEQ ID | FWVLVVVGGVLACYSLLVTVAFIIFWV |
| (Tₘ) domain | NO: 3 | |
| Surface glycoprotein CD8 | SEQ ID | MALPVTALLLPLALLLHAARPSQFRVSPLDRT |
| alpha chain isoform 1 | NO: 4 | WNLGETVELKCQVLLSNPTSGCSWLFQPRGAA |
| precursor | | ASPTFLLYLSQNKPKAAEGLDTQRFSGKRLGDT |
| (NP_001139345.1) | | FVLTLSDFRRENEGYYFCSALSNSIMYFSHFVP |
| | | VFLPAKPTTTPAPRPPTPAPTIASQPLSLRPEACR |
| | | PAAGGAVHTRGLDFACDIYIWAPLAGTCGVLL |
| | | LSLVITLYCNHRNRRRVCKCPRPVVKSGDKPSL |
| | | SARYV |
| 4-1BB costimulatory | SEQ ID | KRGRKKLLYIFKQPFMRPVQTTQEEDGCSCRFP |
| domain | NO: 5 | EEEEGGCEL |
| CD28 costimulatory | SEQ ID | RSKRSRLLHSDYMNMTPRRPGPTRKHYQPYAP |
| domain | NO: 6 | PRDFAAYRS |
| CD3 zeta (ζ) | SEQ ID | RVKFSRSADAPAYKQGQNQLYNELNLGRREEY |
| | NO: 7 | DVLDKRRGRDPEMGGKPRRKNPQEGLYNELQ |
| | | KDKMAEAYSEIGMKGERRRGKGHDGLYQGLS |
| | | TATKDTYDALHMQALPPR |
| P2A peptide | SEQ ID | GSGATNFSLLKQAGDVEENPGP |
| | NO: 8 | |
| (GGGGS)₄ linker | SEQ ID | GGGGSGGGGSGGGGSGGGGS |
| | NO: 9 | |
| hCD34 | SEQ ID | MPRGWTALCLLSLLPSGFMSLDNNGTATPELP |
| | NO: 10 | TQGTFSNVSTNVSYQETTTPSTLGSTSLHPVSQ |
| | | HGNEATTNITETTVKFTSTSVITSVYGNTNSSVQ |
| | | SQTSVISTVFTTPANVSTPETTLKPSLSPGNVSD |
| | | LSTTSTSLATSPTKPYTSSSPILSDIKAEIKCSGIR |
| | | EVKLTQGICLEQNKTSSCAEFKKDRGEGLARV |
| | | LCGEEQADADAGAQVCSLLLAQSEVRPQCLLL |
| | | VLANRTEISSKLQLMKKHQSDLKKLGILDFTEQ |
| | | DVASHQSYSQKTLIALVTSGALLAVLGITGYFL |
| | | MNRRSWSPI |
| Human-Herpes Simplex | SEQ ID | MPRGWTALCLLSLLPSGFMSLDNNGTATPELP |
| Virus-1 (HSV)-thymidine | NO: 11 | TQGTFSNVSTNVSYQETTTPSTLGSTSLHPVSQ |
| kinase (TK) | | HGNEATTNITETTVKFTSTSVITSVYGNTNSSVQ |
| | | SQTSVISTVFTTPANVSTPETTLKPSLSPGNVSD |
| | | LSTTSTSLATSPTKPYTSSSPILSDIKAEIKCSGIR |
| | | EVKLTQGICLEQNKTSSCAEFKKDRGEGLARV |
| | | LCGEEQADADAGAQVCSLLLAQSEVRPQCLLL |
| | | VLANRTEISSKLQLMKKHQSDLKKLGILDFTEQ |
| | | DVASHQSYSQKTLIALVTSGALLAVLGITGYFL |
| | | MNRRSWSPTGEGGGGGDLGGVKLPHLFGKRL |
| | | VEARMASYPCHQHASAFDQAARSRGHSNRRT |
| | | ALRPRRQQEATEVRLEQKMPTLLRVYIDGPHG |
| | | MGKTTTTQLLVALGSRDDIVYVPEPMTYWQV |
| | | LGASETIANIYTTQHRLDQGEISAGDAAVVMTS |
| | | AQITMGMPYAVTDAVLAPHVGGEAGSSHAPPP |
| | | ALTLLLDRHPIAVMLCYPAARYLMGSMTPQAV |
| | | LAFVALIPPTLPGTNIVLGALPEDRHIDRLAKRQ |
| | | RPGERLDLAMLAAIRRVYGLLANTVRYLQGGG |
| | | SWWEDWGQLSGTAVPPQGAEPQSNAGPRPHIG |
| | | DTLFTLFRAPELLAPNGDLYNVFAWALDVLAK |
| | | RLRPMHVFILDYDQSPAGCRDALLQLTSGMVQ |
| | | THVTTPGSIPTICDLARTFAREMGEAN |

TABLE 2
Amino acid sequences of the variable heavy (V_{H}) and
variable light (V_{L}) chains of the scFvs.

| | SEQ ID | |
|---|---|---|
| ScFv sequences | NO: | Amino acid sequence |
| CD2 heavy chain variable | SEQ ID | EVKLEESGAELVKPGASVKLSCRTSGFNlKDTI |
| region (35.1 ATCC ®HB- | NO: 12 | HWVKQRPEQGLKWIGRIDPANGNTKYDPKFQ |
| 222 ™) | | DKATVTADTSSNTAYLQLSSLTSEDTAVYYCV |
| | | TYAYDGNWYFDVWGAGTAVTVSS |
| CD2 light chain variable | SEQ ID | DIKNITQSPSSMYVSLGERVTITCKASQDINSFL |
| region (35.1 ATCC ®HB- | NO: 13 | SWFQQKPGKSPKTLIYRANRLVDGVPSRFSGS |
| 222 ™) | | GSGQDYSLTISSLEYEDMEIYYCLQYDEFPYTF |
| | | GGGTKLEMKR |
| CD2 heavy chain variable | SEQ ID | EVQLEESGAELVRPGTSVKLSCKASGYTFTSY |
| region (OKT 11 | NO: 14 | WMHWIKQRPEQGLEWIGRIDPYDSETHYNEK |
| ATCC ®CRL-8027 ™) | | FKDKAILSVDKSSSTAYIQLSSLTSDDSAVYYC |
| | | SRRDAKYDGYALDYWGQGTSVTVSS |
| CD2 light chain variable | SEQ ID | DI[[J]]VMTQAAPSVPVTPGESVSISCRSSKTLL |
| region (OKT 11 | NO: 15 | HSNGNTYLYWFLQRPGQSPQVLIYRMSNLAS |
| ATCC ®CRL-8027 ™) | | GVPNRFSGSGSETTFTLRISRVEAEDVGIYYCM |
| | | QHLEYPYTFGGGTKLEIER |
| CD3 heavy chain variable | SEQ ID | GSQVQLQQSGAELARPGASVKMSCKASGYTF |
| region (OKT 3) | NO: 16 | TRYTMHWVKQRPGQGLEWIGYINPSRGYTNY |
| | | NQKFKDKATLTTDKSSSTAYMQLSSLTSEDSA |
| | | VYYCARYYDDHYCLDYWGQGTTLTVSS |
| CD3 light chain variable | SEQ ID | QIVLTQSPAIMSASPGEKVTMTCSASSSVSYM |
| region (OKT 3) | NO: 17 | NWYQQKSGTSPKRWIYDTSKLASGVPAHFRG |
| | | SGSGTSYSLTISGMEAEDAATYYCQQWSSNPF |
| | | TFGSGTKLEINR |
| CD3 heavy chain variable | SEQ ID | EVQLVESGGGLVQPGGSLRLSCAASGYSFTGY |
| region (UCHT1) | NO: 18 | TMNWVRQAPGKCLEWVALINPYKGVSTYNQ |
| | | KFKDRFTISVDKSKNTAYLQMNSLRAEDTAV |
| | | YYCARSGYYGDSDWYFDVWGQGTLVTVSS |
| CD3 heavy chain variable | SEQ ID | DIQMTQSPSSLSASVGDRVTITCRASQDIRNYL |
| region (UCHT1) | NO: 19 | NWYQQKPGKAPKLLIYYTSRLESGVPSRFSGS |
| | | GSGTDYTLTISSLQPEDFATYYCQQGNTLPWT |
| | | FGCGTKVEIK |
| CD7 heavy chain variable | SEQ ID | EVQLVESGGGLVKPGGSLKLSCAASGLTFSSY |
| region | NO: 20 | AMSWVRQTPEKRLEWVASISSGGFTYYPDSV |
| | | KGRFTISRDNARNILYLQMSSLRSEDTAMYYC |
| | | ARDEVRGYLDVWGAGTTVTVS |
| CD7 light chain variable | SEQ ID | DIQMTQTTSSLSASLGDRVTISCSASQGISNYL |
| region | NO: 21 | NWYQQKPDGTVKLLIYYTSSLHSGVPSRFSGS |
| | | GSGTDYSLTISNLEPEDIATYYCQQYSKLPYTF |
| | | GGGTKLEIKR |
| FTL3 heavy chain | SEQ ID | EVQLVQSGAEVKKPGASVKVSCKASGYTFTS |
| variable region | NO: 22 | YYMHWVRQAPGQGLEWMGIINPSGGSTSYAQ |
| | | KFQGRVTMTRDTSTSTVYMELSSLRSEDTAVY |
| | | YCARGVGAHDAFDIWGQGTTVTVSS |
| FTL3 light chain variable | SEQ ID | DVVMTQSPLSLPVTPGEPASISCRSSQSLLHSN |
| region | NO: 23 | GNNYLDWYLQKPGQSPQLLIYLGSNRASGVP |
| | | DRFSGSGSDTDFTLQISRVEAEDVGVYYCMQG |
| | | THPAISFGQGTRLEIK |
| FTL3 heavy chain | SEQ ID | EVQLVQSGAEVKKPGSSVKVSCKASGGTFSSY |
| variable region | NO: 24 | AISWVRQAPGQGLEWMGGIIPIFGTANYAQKF |
| | | QGRVTITADKSTSTAYMELSSLRSEDTAVYYC |
| | | ATFALFGFREQAFDIWGQGTTVTVSS |
| FTL3 light chain variable | SEQ ID | DIQMTQSPSSLSASVGDRVTITCRASQSISSYLN |
| region | NO: 25 | WYQQKPGKAPKLLIYAASSLQSGVPSRFSGSG |
| | | SGTDFTLTISSLQPEDLATYYCQQSYSTPFTFGP |
| | | GTKVDIK |
| FTL3 heavy chain | SEQ ID | EVQLVQSGAEVKKPGASVKVSCKASGYTFTS |
| variable region | NO: 26 | YYMHWARQAPGQGLEWMGIINPSGGSTSYAQ |
| | | KFQGRVTMTRDTSTSTVYMELSSLRSEDTAVY |
| | | YCARVVAAAVADYWGQGTLVTVSS |
| FTL3 light chain variable | SEQ ID | DVVMTQSPLSLPVTPGEPASISCRSSQSLLHSN |
| region | NO: 27 | GYNYLDWYLQKPGQSPQLLIYLGSNRASGVP |
| | | DRFSGSGSGTDFTLKISRVEAEDVGVYYCMQS |
| | | LQTPFTFGPGTKVDIK |
| CS1 heavy chain variable | SEQ ID | QVQLQQPGAELVRPGASVKLSCKASGYSFTTY |
| region | NO: 28 | WMNWVKQRPGQGLEWIGMIHPSDSETRL |
| | | NQKFKDKATLTVDKSSSTAYMQLSSPTSEDSA |
| | | VYYCARSTMIATRAMDYWGQGTSVTVSS |
| CS1 light chain variable | SEQ ID | DIVMTQSQKSMSTSVGDRVSITCKASQDVITG |
| region | NO: 29 | VAWYQQKPGQSPKLLIYSASYRYTGVPD |
| | | RFTGSGSGTDFTFTISNVQAEDLAVYYCQQHY |
| | | STPLTFGAGTKLELK |
| CD33 heavy chain | SEQ ID | QVQLQQPGAEVVKPGASVKMSCKASGYTFTS |
| variable region | NO: 30 | YYIHWIKQTPGQGLEWVGVIYPGNDDISYNQK |
| | | FQGKATLTADKSSTTAYMQLSSLTSEDSAVYY |
| | | CAREVRLRYFDVWGQGTTVTVSSSG |
| CD33 light chain variable | SEQ ID | GSEIVLTQSPGSLAVSPGERVTMSCKSSQSVFF |
| region | NO: 31 | SSSQKNYLAWYQQIPGQSPRLLIYWASTRESG |
| | | VPDRFTGSGSGTDFTLTISSVQPEDLAIYYCHQ |
| | | YLSSRTFGQGTKLEIKR |

Mono iNKT-CAR Cells (miNKT)

In certain embodiments, the disclosure provides an engineered iNKT cell comprising a single CAR, that specifically binds CD7, wherein the iNKT cell is deficient in CD7 (e.g., CD7-iNKT-CARΔCD7 cell). In non-limiting examples, the deficiency in CD7 resulted from (a) modification of CD7 expressed by the iNKT cell such that the chimeric antigen receptors no longer specifically binds the modified CD7, (b) modification of the iNKT cell such that expression of CD7 is reduced in the iNKT cell by at least 50%, at least 60%, at least 70%, at least 80%, at least 90% or more, or (c) modification of the iNKT cell such that CD7 is not expressed (e.g., by deletion or disruption of the gene encoding CD7. In further embodiments, the iNKT cell comprises a suicide gene. In non-limiting examples the suicide gene expressed in the CD7-iNKT-CARΔCD7 cells encodes a modified Human-Herpes Simplex Virus-1-thymidine kinase (TK) gene fused in-frame to the extracellular and transmembrane domains of the human CD34 cDNA.

The CAR for a CD7 specific iNKT-CAR cell may be generated by cloning a commercially synthesized anti-CD7 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making CARs specific for other malignant T cell antigens.

Disclosed are embodiments of CAR amino acid sequences that can be expressed on the surface of a genome-edited iNKT cell derived from an iNKT cell.

TABLE 3
Amino Acid Sequences of Mono Chimeric
Antigen Receptors (CARs).

| Mono CAR | SEQ ID | |
|---|---|---|
| Constructs | NO: | Amino acid sequence |
| CD7-CAR-4- | SEQ ID | MALPVTALLLPLALLLHAARPDIQMTQTTSSLSA |
| 1BB_CD34 | NO: 32 | SLGDRVTISCSASQGISNYLNWYQQKPDGTVKLL |
| | | IYYTSSLHSGVPSRFSGSGSGTDYSLTISNLEPEDI |
| | | ATYYCQQYSKLPYTFGGGTKLEIKRGGGGSGGG |
| | | GSGGGGSGGGGSEVQLVESGGGLVKPGGSLKLS |
| | | CAASGLTFSSYAMSWVRQTPEKRLEWVASISSG |
| | | GFTYYPDSVKGRFTISRDNARNILYLQMSSLRSE |
| | | DTAMYYCARDEVRGYLDVWGAGTTVTVSPRAS |
| | | TTTPAPRPPTPAPTIASQPLSLRPEACRPAAGGAV |
| | | HTRGLDFACDFWVLVVVGGVLACYSLLVTVAFI |
| | | IFWVKRGRKKLLYIFKQPFMRPVQTTQEEDGCSC |
| | | RFPEEEEGGCELRVKFSRSADAPAYKQGQNQLY |
| | | NELNLGRREEYDVLDKRRGRDPEMGGKPRRKNP |
| | | QEGLYNELQKDKMAEAYSEIGMKGERRRGKGH |
| | | DGLYQGLSTATKDTYDALHMQALPPRRTDGSGA |
| | | TNFSLLKQAGDVEENPGPVSEAMPRGWTALCLL |
| | | SLLPSGFMSLDNNGTATPELPTQGTFSNVSTNVS |
| | | YQETTTPSTLGSTSLHPVSQHGNEATTNITETTVK |
| | | FTSTSVITSVYGNTNSSVQSQTSVISTVFTTPANVS |
| | | TPETTLKPSLSPGNVSDLSTTSTSLATSPTKPYTSS |
| | | SPILSDIKAEIKCSGIREVKLTQGICLEQNKTSSCA |
| | | EFKKDRGEGLARVLCGEEQADADAGAQVCSLLL |
| | | AQSEVRPQCLLLVLANRTEISSKLQLMKKHQSDL |
| | | KKLGILDFTEQDVASHQSYSQKTLIALVTSGALL |
| | | AVLGITGYFLMNRRSWSPI |
| CD7-CAR-4- | SEQ ID | MALPVTALLLPLALLLHAARPDIQMTQTTSSLSA |
| 1BB_CD34_TK | NO: 33 | SLGDRVTISCSASQGISNYLNWYQQKPDGTVKLL |
| | | IYYTSSLHSGVPSRFSGSGSGTDYSLTISNLEPEDI |
| | | ATYYCQQYSKLPYTFGGGTKLEIKRGGGGSGGG |
| | | GSGGGGSGGGGSEVQLVESGGGLVKPGGSLKLS |
| | | CAASGLTFSSYAMSWVRQTPEKRLEWVASISSG |
| | | GFTYYPDSVKGRFTISRDNARNILYLQMSSLRSE |
| | | DTAMYYCARDEVRGYLDVWGAGTTVTVSPRAS |
| | | TTTPAPRPPTPAPTIASQPLSLRPEACRPAAGGAV |
| | | HTRGLDFACDFWVLVVVGGVLACYSLLVTVAFI |
| | | IFWVKRGRKKLLYIFKQPFMRPVQTTQEEDGCSC |
| | | RFPEEEEGGCELRVKFSRSADAPAYKQGQNQLY |
| | | NELNLGRREEYDVLDKRRGRDPEMGGKPRRKNP |
| | | QEGLYNELQKDKMAEAYSEIGMKGERRRGKGH |
| | | DGLYQGLSTATKDTYDALHMQALPPRRTDGSGA |
| | | TNFSLLKQAGDVEENPGPVSEAMPRGWTALCLL |
| | | SLLPSGFMSLDNNGTATPELPTQGTFSNVSTNVS |
| | | YQETTTPSTLGSTSLHPVSQHGNEATTNITETTVK |
| | | FTSTSVITSVYGNTNSSVQSQTSVISTVFTTPANVS |
| | | TPETTLKPSLSPGNVSDLSTTSTSLATSPTKPYTSS |
| | | SPILSDIKAEIKCSGIREVKLTQGICLEQNKTSSCA |
| | | EFKKDRGEGLARVLCGEEQADADAGAQVCSLLL |
| | | AQSEVRPQCLLLVLANRTEISSKLQLMKKHQSDL |
| | | KKLGILDFTEQDVASHQSYSQKTLIALVTSGALL |
| | | AVLGITGYFLMNRRSWSPTGEGGGGGDLGGVKL |
| | | PHLFGKRLVEARMASYPCHQHASAFDQAARSRG |
| | | HSNRRTALRPRRQQEATEVRLEQKMPTLLRVYID |
| | | GPHGMGKTTTTQLLVALGSRDDIVYVPEPMTYW |
| | | QVLGASETIANIYTTQHRLDQGEISAGDAAVVMT |
| | | SAQITMGMPYAVTDAVLAPHVGGEAGSSHAPPP |
| | | ALTLLLDRHPIAVMLCYPAARYLMGSMTPQAVL |
| | | AFVALIPPTLPGTNIVLGALPEDRHIDRLAKRQRP |
| | | GERLDLAMLAAIRRVYGLLANTVRYLQGGGSW |
| | | WEDWGQLSGTAVPPQGAEPQSNAGPRPHIGDTL |
| | | FTLFRAPELLAPNGDLYNVFAWALDVLAKRLRP |
| | | MHVFILDYDQSPAGCRDALLQLTSGMVQTHVTT |
| | | PGSIPTICDLARTFAREMGEAN |
| CD7-CAR- | SEQ ID | MALPVTALLLPLALLLHAARPDIQMTQTTSSLSA |
| CD28_CD34 | NO: 34 | SLGDRVTISCSASQGISNYLNWYQQKPDGTVKLL |
| | | IYYTSSLHSGVPSRFSGSGSGTDYSLTISNLEPEDI |
| | | ATYYCQQYSKLPYTFGGGTKLEIKRGGGGSGGG |
| | | GSGGGGSGGGGSEVQLVESGGGLVKPGGSLKLS |
| | | CAASGLTFSSYAMSWVRQTPEKRLEWVASISSG |
| | | GFTYYPDSVKGRFTISRDNARNILYLQMSSLRSE |
| | | DTAMYYCARDEVRGYLDVWGAGTTVTVSPRAS |
| | | TTTPAPRPPTPAPTIASQPLSLRPEACRPAAGGAV |
| | | HTRGLDFACDFWVLVVVGGVLACYSLLVTVAFI |
| | | IFWVRSKRSRLLHSDYMNMTPRRPGPTRKHYQP |
| | | YAPPRDFAAYRSRVKFSRSADAPAYKQGQNQLY |
| | | NELNLGRREEYDVLDKRRGRDPEMGGKPRRKNP |
| | | QEGLYNELQKDKMAEAYSEIGMKGERRRGKGH |
| | | DGLYQGLSTATKDTYDALHMQALPPRRTDGSGA |
| | | TNFSLLKQAGDVEENPGPVSEAMPRGWTALCLL |
| | | SLLPSGFMSLDNNGTATPELPTQGTFSNVSTNVS |
| | | YQETTTPSTLGSTSLHPVSQHGNEATTNITETTVK |
| | | FTSTSVITSVYGNTNSSVQSQTSVISTVFTTPANVS |
| | | TPETTLKPSLSPGNVSDLSTTSTSLATSPTKPYTSS |
| | | SPILSDIKAEIKCSGIREVKLTQGICLEQNKTSSCA |
| | | EFKKDRGEGLARVLCGEEQADADAGAQVCSLLL |
| | | AQSEVRPQCLLLVLANRTEISSKLQLMKKHQSDL |
| | | KKLGILDFTEQDVASHQSYSQKTLIALVTSGALL |
| | | AVLGITGYFLMNRRSWSPI |
| CD7-CAR- | SEQ ID | MALPVTALLLPLALLLHAARPDIQMTQTTSSLSA |
| CD28_CD34_TK | NO: 35 | SLGDRVTISCSASQGISNYLNWYQQKPDGTVKLL |
| | | IYYTSSLHSGVPSRFSGSGSGTDYSLTISNLEPEDI |
| | | ATYYCQQYSKLPYTFGGGTKLEIKRGGGGSGGG |
| | | GSGGGGSGGGGSEVQLVESGGGLVKPGGSLKLS |
| | | CAASGLTFSSYAMSWVRQTPEKRLEWVASISSG |
| | | GFTYYPDSVKGRFTISRDNARNILYLQMSSLRSE |
| | | DTAMYYCARDEVRGYLDVWGAGTTVTVSPRAS |
| | | TTTPAPRPPTPAPTIASQPLSLRPEACRPAAGGAV |
| | | HTRGLDFACDFWVLVVVGGVLACYSLLVTVAFI |
| | | IFWVRSKRSRLLHSDYMNMTPRRPGPTRKHYQP |
| | | YAPPRDFAAYRSRVKFSRSADAPAYKQGQNQLY |
| | | NELNLGRREEYDVLDKRRGRDPEMGGKPRRKNP |
| | | QEGLYNELQKDKMAEAYSEIGMKGERRRGKGH |
| | | DGLYQGLSTATKDTYDALHMQALPPRRTDGSGA |
| | | TNFSLLKQAGDVEENPGPVSEAMPRGWTALCLL |
| | | SLLPSGFMSLDNNGTATPELPTQGTFSNVSTNVS |
| | | YQETTTPSTLGSTSLHPVSQHGNEATTNITETTVK |
| | | FTSTSVITSVYGNTNSSVQSQTSVISTVFTTPANVS |
| | | TPETTLKPSLSPGNVSDLSTTSTSLATSPTKPYTSS |
| | | SPILSDIKAEIKCSGIREVKLTQGICLEQNKTSSCA |
| | | EFKKDRGEGLARVLCGEEQADADAGAQVCSLLL |
| | | AQSEVRPQCLLLVLANRTEISSKLQLMKKHQSDL |
| | | KKLGILDFTEQDVASHQSYSQKTLIALVTSGALL |
| | | AVLGITGYFLMNRRSWSPTGEGGGGGDLGGVKL |
| | | PHLFGKRLVEARMASYPCHQHASAFDQAARSRG |
| | | HSNRRTALRPRRQQEATEVRLEQKMPTLLRVYID |
| | | GPHGMGKTTTTQLLVALGSRDDIVYVPEPMTYW |
| | | QVLGASETIANIYTTQHRLDQGEISAGDAAVVMT |
| | | SAQITMGMPYAVTDAVLAPHVGGEAGSSHAPPP |
| | | ALTLLLDRHPIAVMLCYPAARYLMGSMTPQAVL |
| | | AFVALIPPTLPGTNIVLGALPEDRHIDRLAKRQRP |
| | | GERLDLAMLAAIRRVYGLLANTVRYLQGGGSW |
| | | WEDWGQLSGTAVPPQGAEPQSNAGPRPHIGDTL |
| | | FTLFRAPELLAPNGDLYNVFAWALDVLAKRLRP |
| | | MHVFILDYDQSPAGCRDALLQLTSGMVQTHVTT |
| | | PGSIPTICDLARTFAREMGEAN |
| CD79B-CAR- | SEQ ID | MALPVTALLLPLALLLHAARPGSDIQLTQSPSSLS |
| CD28_CD34 | NO: 36 | ASVGDRVTITCKASQSVDYEGDSFLNWYQQKPG |
| | | KAPKLLIYAASNLESGVPSRFSGSGSGTDFTLTISS |
| | | LQPEDFATYYCQQSNEDPLTFGQGTKVEIKRGGG |
| | | GSGGGGSGGGGSGGGGSGGGGSEVQLVESGGG |
| | | LVQPGGSLRLSCAASGYTFSSYWIEWVRQAPGK |
| | | GLEWIGEILPGGGDTNYNEIFKGRATFSADTSKN |
| | | TAYLQMNSLRAEDTAVYYCTRRVPIRLDYWGQ |
| | | GTLVTVSSPRASTTTPAPRPPTPAPTIASQPLSLRP |
| | | EACRPAAGGAVHTRGLDFACDFWVLVVVGGVL |
| | | ACYSLLVTVAFIIFWVRSKRSRLLHSDYMNMTPR |
| | | RPGPTRKHYQPYAPPRDFAAYRSRVKFSRSADAP |
| | | AYKQGQNQLYNELNLGRREEYDVLDKRRGRDP |
| | | EMGGKPRRKNPQEGLYNELQKDKMAEAYSEIG |
| | | MKGERRRGKGHDGLYQGLSTATKDTYDALHMQ |
| | | ALPPRRTDGSGATNFSLLKQAGDVEENPGPVSEA |
| | | MPRGWTALCLLSLLPSGFMSLDNNGTATPELPTQ |
| | | GTFSNVSTNVSYQETTTPSTLGSTSLHPVSQHGN |
| | | EATTNITETTVKFTSTSVITSVYGNTNSSVQSQTS |
| | | VISTVFTTPANVSTPETTLKPSLSPGNVSDLSTTST |
| | | SLATSPTKPYTSSSPILSDIKAEIKCSGIREVKLTQ |
| | | GICLEQNKTSSCAEFKKDRGEGLARVLCGEEQA |
| | | DADAGAQVCSLLLAQSEVRPQCLLLVLANRTEIS |
| | | SKLQLMKKHQSDLKKLGILDFTEQDVASHQSYS |
| | | QKTLIALVTSGALLAVLGITGYFLMNRRSWSPTG |
| | | EGGGGGFKRDLGGVKLPHLFGKRLVEARMASYP |
| | | CHQHASAFDQAARSRGHSNRRTALRPRRQQEAT |
| | | EVRLEQKMPTLLRVYIDGPHGMGKTTTTQLLVA |
| | | LGSRDDIVYVPEPMTYWQVLGASETIANIYTTQH |
| | | RLDQGEISAGDAAVVMTSAQITMGMPYAVTDA |
| | | VLAPHVGGEAGSSHAPPPALTLLLDRHPIAVMLC |
| | | YPAARYLMGSMTPQAVLAFVALIPPTLPGTNIVL |
| | | GALPEDRHIDRLAKRQRPGERLDLAMLAAIRRV |
| | | YGLLANTVRYLQGGGSWWEDWGQLSGTAVPPQ |
| | | GAEPQSNAGPRPHIGDTLFTLFRAPELLAPNGDL |
| | | YNVFAWALDVLAKRLRPMHVFILDYDQSPAGCR |
| | | DALLQLTSGMVQTHVTTPGSIPTICDLARTFARE |
| | | MGEAN |
| CD2-CAR- | SEQ ID | MALPVTALLLPLALLLHAARPDIVMTQAAPSVPV |
| CD28_CD34 | NO: 37 | TPGESVSISCRSSKTLLHSNGNTYLYWFLQRPGQ |
| | | SPQVLIYRMSNLASGVPNRFSGSGSETTFTLRISR |
| | | VEAEDVGIYYCMQHLEYPYTFGGGTKLEIERGG |
| | | GGSGGGGSGGGGSGGGGSEVQLEESGAELVRPG |
| | | TSVKLSCKASGYTFTSYWMHWIKQRPEQGLEWI |
| | | GRIDPYDSETHYNEKFKDKAILSVDKSSSTAYIQL |
| | | SSLTSDDSAVYYCSRRDAKYDGYALDYWGQGT |
| | | SVTVSSPRASTTTPAPRPPTPAPTIASQPLSLRPEA |
| | | CRPAAGGAVHTRGLDFACDFWVLVVVGGVLAC |
| | | YSLLVTVAFIIFWVRSKRSRLLHSDYMNMTPRRP |
| | | GPTRKHYQPYAPPRDFAAYRSRVKFSRSADAPA |
| | | YKQGQNQLYNELNLGRREEYDVLDKRRGRDPE |
| | | MGGKPRRKNPQEGLYNELQKDKMAEAYSEIGM |
| | | KGERRRGKGHDGLYQGLSTATKDTYDALHMQA |
| | | LPPRRTDGSGATNFSLLKQAGDVEENPGPVSEA |
| | | MPRGWTALCLLSLLPSGFMSLDNNGTATPELPTQ |
| | | GTFSNVSTNVSYQETTTPSTLGSTSLHPVSQHGN |
| | | EATTNITETTVKFTSTSVITSVYGNTNSSVQSQTS |
| | | VISTVFTTPANVSTPETTLKPSLSPGNVSDLSTTST |
| | | SLATSPTKPYTSSSPILSDIKAEIKCSGIREVKLTQ |
| | | GICLEQNKTSSCAEFKKDRGEGLARVLCGEEQA |
| | | DADAGAQVCSLLLAQSEVRPQCLLLVLANRTEIS |
| | | SKLQLMKKHQSDLKKLGILDFTEQDVASHQSYS |
| | | QKTLIALVTSGALLAVLGITGYFLMNRRSWSPI |
| CD2-CAR-4- | SEQ ID | MALPVTALLLPLALLLHAARPDIVMTQAAPSVPV |
| 1BB_CD34 | NO: 38 | TPGESVSISCRSSKTLLHSNGNTYLYWFLQRPGQ |
| | | SPQVLIYRMSNLASGVPNRFSGSGSETTFTLRISR |
| | | VEAEDVGIYYCMQHLEYPYTFGGGTKLEIERGG |
| | | GGSGGGGSGGGGSGGGGSEVQLEESGAELVRPG |
| | | TSVKLSCKASGYTFTSYWMHWIKQRPEQGLEWI |
| | | GRIDPYDSETHYNEKFKDKAILSVDKSSSTAYIQL |
| | | SSLTSDDSAVYYCSRRDAKYDGYALDYWGQGT |
| | | SVTVSSPRASTTTPAPRPPTPAPTIASQPLSLRPEA |
| | | CRPAAGGAVHTRGLDFACDFWVLVVVGGVLAC |
| | | YSLLVTVAFIIFWVKRGRKKLLYIFKQPFMRPVQ |
| | | TTQEEDGCSCRFPEEEEGGCELRVKFSRSADAPA |
| | | YKQGQNQLYNELNLGRREEYDVLDKRRGRDPE |
| | | MGGKPRRKNPQEGLYNELQKDKMAEAYSEIGM |
| | | KGERRRGKGHDGLYQGLSTATKDTYDALHMQA |
| | | LPPRRTDGSGATNFSLLKQAGDVEENPGPVSEA |
| | | MPRGWTALCLLSLLPSGFMSLDNNGTATPELPTQ |
| | | GTFSNVSTNVSYQETTTPSTLGSTSLHPVSQHGN |
| | | EATTNITETTVKFTSTSVITSVYGNTNSSVQSQTS |
| | | VISTVFTTPANVSTPETTLKPSLSPGNVSDLSTTST |
| | | SLATSPTKPYTSSSPILSDIKAEIKCSGIREVKLTQ |
| | | GICLEQNKTSSCAEFKKDRGEGLARVLCGEEQA |
| | | DADAGAQVCSLLLAQSEVRPQCLLLVLANRTEIS |
| | | SKLQLMKKHQSDLKKLGILDFTEQDVASHQSYS |
| | | QKTLIALVTSGALLAVLGITGYFLMNRRSWSPI |
| CD3-CD28- | SEQ ID | MALPVTALLLPLALLLHAARPGSQVQLQQSGAE |
| CD34 | NO: 39 | LARPGASVKMSCKASGYTFTRYTMHWVKQRPG |
| | | QGLEWIGYINPSRGYTNYNQKFKDKATLTTDKSS |
| | | STAYMQLSSLTSEDSAVYYCARYYDDHYCLDY |
| | | WGQGTTLTVSSGGGGSGGGGSGGGGSGGGGSQI |
| | | VLTQSPAIMSASPGEKVTMTCSASSSVSYMNWY |
| | | QQKSGTSPKRWIYDTSKLASGVPAHFRGSGSGTS |
| | | YSLTISGMEAEDAATYYCQQWSSNPFTFGSGTKL |
| | | EINRPRASTTTPAPRPPTPAPTIASQPLSLRPEACR |
| | | PAAGGAVHTRGLDFACDFWVLVVVGGVLACYS |
| | | LLVTVAFIIFWVRSKRSRLLHSDYMNMTPRRPGP |
| | | TRKHYQPYAPPRDFAAYRSRVKFSRSADAPAYK |
| | | QGQNQLYNELNLGRREEYDVLDKRRGRDPEMG |
| | | GKPRRKNPQEGLYNELQKDKMAEAYSEIGMKG |
| | | ERRRGKGHDGLYQGLSTATKDTYDALHMQALP |
| | | PRRTDGSGATNFSLLKQAGDVEENPGPVSEAMP |
| | | RGWTALCLLSLLPSGFMSLDNNGTATPELPTQGT |
| | | FSNVSTNVSYQETTTPSTLGSTSLHPVSQHGNEA |
| | | TTNITETTVKFTSTSVITSVYGNTNSSVQSQTSVIS |
| | | TVFTTPANVSTPETTLKPSLSPGNVSDLSTTSTSL |
| | | ATSPTKPYTSSSPILSDIKAEIKCSGIREVKLTQGIC |
| | | LEQNKTSSCAEFKKDRGEGLARVLCGEEQADAD |
| | | AGAQVCSLLLAQSEVRPQCLLLVLANRTEISSKL |
| | | QLMKKHQSDLKKLGILDFTEQDVASHQSYSQKT |
| | | LIALVTSGALLAVLGITGYFLMNRRSWSPI |

In a similar manner, other mono-iNKT cells may be constructed and are given below in Table 4.

TABLE 4
Mono-iNKT cells

| | Antigen Target | Antigen Deletion/ |
|---|---|---|
| Example | of iNKT-CAR | Suppression |
| M1 | CD2 | — |
| M2 | CD3ε | — |
| M3 | CD4 | — |
| M4 | CD5 | — |
| M5 | CD7 | — |
| M6 | TRAC | — |
| M7 | TCRβ | — |
| M8 | CD2 | CD2 |
| M9 | CD3ε | CD3ε |
| M10 | CD4 | CD4 |
| M11 | CD5 | CD5 |
| M12 | CD7 | CD7 |
| M13 | TRAC | TRAC |
| M14 | TCRβ | TCRβ |
| M15 | BCMA | |
| M16 | CS1 | |
| M17 | CD19 | |
| M18 | CS1 | CS1 |
| M19 | CD38 | CD38 |

Tandem iNKT-CAR Cells

In certain embodiments, the disclosure provides an engineered iNKT cell comprising a tandem CAR (tCAR), i.e., two scFv sharing a single intracellular domain, that specifically binds CD7 and CD2, wherein the iNKT cell is deficient in CD7 and CD2 (e.g., CD7×CD2-iNKT-tCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-tCARΔCD7ΔCD2 cells encodes a modified Human-Herpes Simplex Virus-1-thymidine kinase (TK) gene fused in-frame to the extracellular and transmembrane domains of the human CD34 cDNA.

A tCAR for a genome-edited, tandem iNKT-CAR cell, i.e., CD7*CD2-iNKT-tCARΔCD7ΔCD2, may be generated by cloning a commercially synthesized anti-CD7 single chain variable fragment (scFv) and an anti-CD2 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making tCARs specific for other malignant T cell antigens.

### Linear Tandem CAR Constructs

In one embodiment of a linear tandem CAR construct, the first extracellular ligand-binding domain comprises a single chain antibody fragment (scFv), comprising the heavy (V_{H}) and the light (V_{L}) variable fragment, designated V_{H}1 and V_{L}1, and joined by a linker (e.g., GGGGS)₂₋₆ (SEQ ID NO:447). The second extracellular ligand-binding domain antigen recognition comprises a single chain antibody fragment (scFv), comprising the light (V_{L}) and the heavy (V_{H}) variable fragment, designated V_{L}2 and V_{H}2, and joined by a linker (e.g., GGGGS)₂₋₆ (SEQ ID NO:447).

In a second embodiment of a linear tandem CAR construct, the first extracellular ligand-binding domain comprises a single chain antibody fragment (scFv), comprising the heavy (V_{H}) and the light (V_{L}) variable fragment, designated V_{H}2 and V_{L}2, and joined by a linker (e.g., GGGGS)₂₋₆ (SEQ ID NO:447). The second extracellular ligand-binding domain antigen recognition comprises a single chain antibody fragment (scFv), comprising the light (V_{L}) and the heavy (V_{H}) variable fragment, designated V_{L}1 and V_{H}1, and joined by a linker (e.g., GGGGS)₂₋₆ (SEQ ID NO:447).

In a third embodiment of a linear tandem CAR construct, the first extracellular ligand-binding domain comprises a single chain antibody fragment (scFv), comprising the heavy (VL) and the light (VH) variable fragment, designated V_{L}1 and V_{H}1, and joined by a linker (e.g., GGGGS)₂₋₆ (SEQ ID NO:447). The second extracellular ligand-binding domain antigen recognition comprises a single chain antibody fragment (scFv), comprising the light (V_{H}) and the heavy (V_{L}) variable fragment, designated V_{H}2 and V_{L}2, and joined by a linker (e.g., GGGGS)₂₋₆ (SEQ ID NO:447).

In a fourth embodiment of a linear tandem CAR construct, the first extracellular ligand-binding domain comprises a single chain antibody fragment (scFv), comprising the heavy (V_{L}) and the light (V_{H}) variable fragment, designated V_{L}2 and V_{H}2, and joined by a linker (e.g., GGGGS)₂₋₆ (SEQ ID NO:447). The second extracellular ligand-binding domain antigen recognition comprises a single chain antibody fragment (scFv), comprising the light (V_{H}) and the heavy (V_{L}) variable fragment, designated V_{H}1 and V_{L}1, and joined by a linker (e.g., GGGGS)₂₋₆ (SEQ ID NO:447).

For each of the linear tandem CAR construct embodiments, the first and second extracellular ligand-binding domains targets a surface molecule, i.e., an antigen expressed on a malignant T cell is selected from, but not limited to, BCMA, CS1, CD38, CD138, CD19, CD33, CD123, CD371, CD117, CD135, Tim-3, CD5, CD7, CD2, CD4, CD3ε, CD79A, CD79B, APRIL, CD56, and CD1a, TRAC, and TCRβ.

Further examples of linear tandem CAR are given below in Table 5.

TABLE 5
Tandem CARs and iNKT cells (Linear or Hairpin).

| | Antigen Target | Antigen Deletion/ |
|---|---|---|
| Example | of iNKT-CAR | Suppression |
| T1 | CD2 × CD3ε | — |
| T2 | CD2 × CD3ε | CD2 |
| T3 | CD2 × CD3ε | CD3ε |
| T4 | CD2 × CD3ε | CD2 and CD3ε |
| T5 | CD2 × CD4 | — |
| T6 | CD2 × CD4 | CD2 |
| T7 | CD2 × CD4 | CD4 |
| T8 | CD2 × CD4 | CD2 and CD4 |
| T9 | CD2 × CD5 | — |
| T10 | CD2 × CD5 | CD2 |
| T11 | CD2 × CD5 | CD5 |
| T12 | CD2 × CD5 | CD2 and CD5 |
| T13 | CD2 × CD7 | — |
| T14 | CD2 × CD7 | CD2 |
| T15 | CD2 × CD7 | CD7 |
| T16 | CD2 × CD7 | CD2 and CD7 |
| T17 | CD3ε × CD4 | — |
| T18 | CD3ε × CD4 | CD3ε |
| T19 | CD3ε × CD4 | CD4 |
| T20 | CD3ε × CD4 | CD3ε and CD4 |
| T21 | CD3ε × CD5 | — |
| T22 | CD3ε × CD5 | CD3ε |
| T23 | CD3ε × CD5 | CD5 |
| T24 | CD3ε × CD5 | CD3ε and CD5 |
| T25 | CD3ε × CD7 | — |
| T26 | CD3ε × CD7 | CD3ε |
| T27 | CD3ε × CD7 | CD7 |
| T28 | CD3ε × CD7 | CD3ε and CD7 |
| T29 | CD4 × CD5 | — |
| T30 | CD4 × CD5 | CD4 |
| T31 | CD4 × CD5 | CD5 |
| T32 | CD4 × CD5 | CD4 and CD5 |
| T33 | CD4 × CD7 | — |
| T34 | CD4 × CD7 | CD4 |
| T35 | CD4 × CD7 | CD7 |
| T36 | CD4 × CD7 | CD5 and CD7 |
| T37 | CD5 × CD7 | — |
| T38 | CD5 × CD7 | CD5 |
| T39 | CD5 × CD7 | CD7 |
| T40 | CD5 × CD7 | CD5 and CD7 |
| T41 | TRAC × CD2 | — |
| T42 | TRAC × CD2 | TRAC |
| T43 | TRAC × CD2 | CD2 |
| T44 | TRAC × CD2 | TRAC and CD2 |
| T45 | TRAC × CD3ε | — |
| T46 | TRAC × CD3ε | TRAC |
| T47 | TRAC × CD3ε | CD3ε |
| T48 | TRAC × CD3ε | TRAC and CD3ε |
| T49 | TRAC × CD4 | — |
| T50 | TRAC × CD4 | TRAC |
| T51 | TRAC × CD4 | CD4 |
| T52 | TRAC × CD4 | TRAC and CD4 |
| T53 | TRAC × CD5 | — |
| T54 | TRAC × CD5 | TRAC |
| T55 | TRAC × CD5 | CD5 |
| T56 | TRAC × CD5 | TRAC and CD5 |
| T57 | TRAC × CD7 | — |
| T58 | TRAC × CD7 | TRAC |
| T59 | TRAC × CD7 | CD7 |
| T60 | TRAC × CD7 | TRAC and CD7 |
| T61 | TCRβ × CD2 | — |
| T62 | TCRβ × CD2 | TCRβ |
| T63 | TCRβ × CD2 | CD2 |
| T64 | TCRβ × CD2 | TCRβ and CD2 |
| T65 | TCRβ × CD3ε | — |
| T66 | TCRβ × CD3ε | TCRβ |
| T67 | TCRβ × CD3ε | CD3ε |
| T68 | TCRβ × CD3ε | TCRβ and CD3ε |
| T69 | TCRβ × CD4 | — |
| T70 | TCRβ × CD4 | TCRβ |
| T71 | TCRβ × CD4 | CD4 |
| T72 | TCRβ × CD4 | TCRβ and CD4 |
| T73 | TCRβ × CD5 | — |
| T74 | TCRβ × CD5 | TCRβ |
| T75 | TCRβ × CD5 | CD5 |
| T76 | TCRβ × CD5 | TCRβ and CD5 |
| T77 | TCRβ × CD7 | — |
| T78 | TCRβ × CD7 | TCRβ |
| T79 | TCRβ × CD7 | CD7 |
| T80 | TCRβ × CD7 | TCRβ and CD7 |
| T81 | BCMA × CS1 | — |
| T82 | BCMA × CS1 | CS1 |
| T83 | BCMA × CD19 | — |
| T84 | BCMA × CD38 | — |
| T85 | BCMA × CD38 | CD38 |
| T86 | CS1 × CD19 | — |
| T87 | CS1 × CD19 | CS1 |
| T88 | CS1 × CD38 | — |
| T89 | CS1 × CD38 | CS1 |
| T90 | CS1 × CD38 | CD38 |
| T91 | CS1 × CD38 | CS1 and CD38 |
| T92 | CD19 × CD38 | — |
| T93 | CD19 × CD38 | CD38 |
| T94 | APRIL × CS1 | — |
| T95 | APRIL × CS1 | CS1 |
| T96 | APRIL × BCMA | — |
| T97 | APRIL × CD19 | — |
| T98 | APRIL × CD38 | — |
| T99 | APRIL × CD38 | CD38 |

For example, provided herein are linear tandem CAR constructs which may incorporate the V_{H} and V_{L} domains of scFvs targeting any of the antigen pairs provided in the Examples in Table 5 above.

TABLE 6
Linear Tandem CAR Constructs.

| 6-I | 6-II | 6-III | 6-IV | 6-V | 6-VI | 6-VII | 6-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447)₎ | NO: 447) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 6-IX | 6-X | 6-XI | 6-XII | 6-XIII | 6-XIV | 6-XV | 6-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 6-XVII | 6-XVIII | 6-XIX | 6-XX | 6-XXI | 6-XXII | 6-XXIII | 6-XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| | | | 6- | | | | |
| 6-XXV | 6-XXVI | 6-XXVII | XXVIII | 6-XIX | 6-XXX | 6-XXXI | 6-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |

Hairpin Tandem CAR Constructs

In one embodiment of a hairpin tandem CAR construct, the first extracellular ligand-binding domain comprises a single chain antibody fragment (scFv), comprising two heavy chain variable fragments, designated V_{H}1 and V_{H}2, and joined by a linker (e.g., GGGGS)₂₋₆ (SEQ ID NO:447). The second extracellular ligand-binding domain antigen recognition comprises a single chain antibody fragment (scFv), comprising two light chain variable fragments, designated V_{L}2 and V_{L}1, and joined by a linker (e.g., GGGGS)₂₋₆ (SEQ ID NO:447).

In a second embodiment of a hairpin tandem CAR construct, the first extracellular ligand-binding domain comprises a single chain antibody fragment (scFv), comprising two heavy chain variable fragments, designated V_{H}2 and V_{H}1, and joined by a linker (e.g., GGGGS)₂₋₆ (SEQ ID NO:447). The second extracellular ligand-binding domain antigen recognition comprises a single chain antibody fragment (scFv), comprising two light chain variable fragments, designated V_{L}1 and V_{L}2, and joined by a linker (e.g., GGGGS)₂₋₆ (SEQ ID NO:447).

In a third embodiment of a hairpin tandem CAR construct, the first extracellular ligand-binding domain comprises a single chain antibody fragment (scFv), comprising two light chain variable fragments, designated V_{L}1 and V_{L}2, and joined by a linker (e.g., GGGGS)₂₋₆ (SEQ ID NO:447). The second extracellular ligand-binding domain antigen recognition comprises a single chain antibody fragment (scFv), comprising two heavy chain variable fragments, designated V_{H}2 and V_{H}1, and joined by a linker (e.g., GGGGS)₂₋₆ (SEQ ID NO:447).

In a fourth embodiment of a hairpin tandem CAR construct, the first extracellular ligand-binding domain comprises a single chain antibody fragment (scFv), comprising two light chain variable fragments, designated V_{L}2 and V_{L}1, and joined by a linker (e.g., GGGGS)₂₋₆ (SEQ ID NO:447). The second extracellular ligand-binding domain antigen recognition comprises a single chain antibody fragment (scFv), comprising two heavy chain variable fragments, designated V_{H}1 and V_{H}2, and joined by a linker (e.g., GGGGS)₂₋₆ (SEQ ID NO:447).

For each of the hairpin tandem CAR construct embodiments, the first and second extracellular ligand-binding domains targets a surface molecule, i.e., an antigen expressed on a malignant T cell is selected from, but not limited to, BCMA, CS1, CD38, CD138, CD19, CD33, CD123, CD371, CD117, CD135, Tim-3, CD5, CD7, CD2, CD4, CD3ε, CD79A, CD79B, APRIL, CD56, and CD1a, TRAC, and TCRβ.

Additional examples of hairpin tandem CARs are given above in Table 5.

Furthermore, provided herein are CAR constructs and iNKT cells which may incorporate the V_{H} and V_{L} domains of scFvs targeting (1) CD2 and CD3; and (2) CD2 and CD7 and are provided below in Table 7.

TABLE 7
Amino Acid Sequences of Hairpin Tandem
Chimeric Antigen Receptors (CARs).

| Hairpin | | |
|---|---|---|
| Tandem | SEQ ID | |
| CAR Constructs | NO: | Amino acid sequence |
| OKT3 V_{L}- | SEQ ID | MALPVTALLLPLALLLHAARPQIVLTQSPAIM |
| CD2 V_{L}- | NO: 41 | SASPGEKVTMTCSASSSVSYMNWYQQKSGTS |
| CD2 V_{H}- | | PKRWIYDTSKLASGVPAHFRGSGSGTSYSLTI |
| OKT3 V_{H} | | SGMEAEDAATYYCQQWSSNPFTFGSGTKLEI |
| | | NRGGGGSGGGGSGGGGSGGGGSDIKNITQSP |
| | | SSMYVSLGERVTITCKASQDINSFLSWFQQKP |
| | | GKSPKTLIYRANRLVDGVPSRFSGSGSGQDYS |
| | | LTISSLEYEDMEIYYCLQYDEFPYTFGGGTKL |
| | | EMKRGGGGSGGGGSGGGGSGGGGSGGGGS |
| | | GGGGSGGGGSGGGGSGGGGSGGGGSEVKLE |
| | | ESGAELVKPGASVKLSCRTSGFN1KDTIHWVK |
| | | QRPEQGLKWIGRIDPANGNTKYDPKFQDKAT |
| | | VTADTSSNTAYLQLSSLTSEDTAVYYCVTYA |
| | | YDGNWYFDVWGAGTAVTVSSGGGGSGGGG |
| | | SGGGGSGGGGSGSQVQLQQSGAELARPGAS |
| | | VKMSCKASGYTFTRYTMHWVKQRPGQGLE |
| | | WIGYINPSRGYTNYNQKFKDKATLTTDKSSS |
| | | TAYMQLSSLTSEDSAVYYCARYYDDHYCLD |
| | | YWGQGTTLTVSSPRASTTTPAPRPPTPAPTIAS |
| | | QPLSLRPEACRPAAGGAVHTRGLDFACDFWV |
| | | LVVVGGVLACYSLLVTVAFIIFWVRSKRSRLL |
| | | HSDYMNMTPRRPGPTRKHYQPYAPPRDFAA |
| | | YRSRVKFSRSADAPAYKQGQNQLYNELNLG |
| | | RREEYDVLDKRRGRDPEMGGKPRRKNPQEG |
| | | LYNELQKDKMAEAYSEIGMKGERRRGKGHD |
| | | GLYQGLSTATKDTYDALHMQALPPRRTDGS |
| | | GATNFSLLKQAGDVEENPGPVSEAMPRGWT |
| | | ALCLLSLLPSGFMSLDNNGTATPELPTQGTFS |
| | | NVSTNVSYQETTTPSTLGSTSLHPVSQHGNEA |
| | | TTNITETTVKFTSTSVITSVYGNTNSSVQSQTS |
| | | VISTVFTTPANVSTPETTLKPSLSPGNVSDLST |
| | | TSTSLATSPTKPYTSSSPILSDIKAEIKCSGIREV |
| | | KLTQGICLEQNKTSSCAEFKKDRGEGLARVL |
| | | CGEEQADADAGAQVCSLLLAQSEVRPQCLLL |
| | | VLANRTEISSKLQLMKKHQSDLKKLGILDFTE |
| | | QDVASHQSYSQKTLIALVTSGALLAVLGITGY |
| | | FLMNRRSWSPI |
| CD3 V_{L}- | SEQ ID | MALPVTALLLPLALLLHAARPDIQMTQSPSSL |
| CD2 V_{L}- | NO: 42 | SASVGDRVTITCRASQDIRNYLNWYQQKPGK |
| CD2-V_{H}- | | APKLLIYYTSRLESGVPSRFSGSGSGTDYTLTI |
| CD3 V_{H} | | SSLQPEDFATYYCQQGNTLPWTFGCGTKVEI |
| | | KGGGGSGGGGSGGGGSGGGGSDIKNITQSPS |
| | | SMYVSLGERVTITCKASQDINSFLSWFQQKPG |
| | | KSPKTLIYRANRLVDGVPSRFSGSGSGQDYSL |
| | | TISSLEYEDMEIYYCLQYDEFPYTFGGGTKLE |
| | | MKRGGGGSGGGGSGGGGSGGGGSGGGGSG |
| | | GGGSGGGGSGGGGSGGGGSGGGGSEVKLEE |
| | | SGAELVKPGASVKLSCRTSGFN1KDTIHWVK |
| | | QRPEQGLKWIGRIDPANGNTKYDPKFQDKAT |
| | | VTADTSSNTAYLQLSSLTSEDTAVYYCVTYA |
| | | YDGNWYFDVWGAGTAVTVSSGGGGSGGGG |
| | | SGGGGSGGGGSEVQLVESGGGLVQPGGSLRL |
| | | SCAASGYSFTGYTMNWVRQAPGKCLEWVAL |
| | | INPYKGVSTYNQKFKDRFTISVDKSKNTAYL |
| | | QMNSLRAEDTAVYYCARSGYYGDSDWYFD |
| | | VWGQGTLVTVSSPRASTTTPAPRPPTPAPTIA |
| | | SQPLSLRPEACRPAAGGAVHTRGLDFACDFW |
| | | VLVVVGGVLACYSLLVTVAFIIFWVRSKRSR |
| | | LLHSDYMNMTPRRPGPTRKHYQPYAPPRDFA |
| | | AYRSRVKFSRSADAPAYKQGQNQLYNELNL |
| | | GRREEYDVLDKRRGRDPEMGGKPRRKNPQE |
| | | GLYNELQKDKMAEAYSEIGMKGERRRGKGH |
| | | DGLYQGLSTATKDTYDALHMQALPPRRTDG |
| | | SGATNFSLLKQAGDVEENPGPVSEAMPRGWT |
| | | ALCLLSLLPSGFMSLDNNGTATPELPTQGTFS |
| | | NVSTNVSYQETTTPSTLGSTSLHPVSQHGNEA |
| | | TTNITETTVKFTSTSVITSVYGNTNSSVQSQTS |
| | | VISTVFTTPANVSTPETTLKPSLSPGNVSDLST |
| | | TSTSLATSPTKPYTSSSPILSDIKAEIKCSGIREV |
| | | KLTQGICLEQNKTSSCAEFKKDRGEGLARVL |
| | | CGEEQADADAGAQVCSLLLAQSEVRPQCLLL |
| | | VLANRTEISSKLQLMKKHQSDLKKLGILDFTE |
| | | QDVASHQSYSQKTLIALVTSGALLAVLGITGY |
| | | FLMNRRSWSPI |
| CD2 V_{L}- | SEQ ID | MALPVTALLLPLALLLHAARPDIKNITQSPSS |
| CD3 V_{L}- | NO: 43 | MYVSLGERVTITCKASQDINSFLSWFQQKPG |
| CD3 V_{H}- | | KSPKTLIYRANRLVDGVPSRFSGSGSGQDYSL |
| CD2-V_{H} | | TISSLEYEDMEIYYCLQYDEFPYTFGGGTKLE |
| | | MKRGGGGSGGGGSGGGGSGGGGSDIQMTQS |
| | | PSSLSASVGDRVTITCRASQDIRNYLNWYQQ |
| | | KPGKAPKLLIYYTSRLESGVPSRFSGSGSGTD |
| | | YTLTISSLQPEDFATYYCQQGNTLPWTFGCGT |
| | | KVEIKGGGGSGGGGSGGGGSGGGGSGGGGS |
| | | GGGGSGGGGSGGGGSGGGGSGGGGSEVQLV |
| | | ESGGGLVQPGGSLRLSCAASGYSFTGYTMN |
| | | WVRQAPGKCLEWVALINPYKGVSTYNQKFK |
| | | DRFTISVDKSKNTAYLQMNSLRAEDTAVYYC |
| | | ARSGYYGDSDWYFDVWGQGTLVTVSSGGG |
| | | GSGGGGSGGGGSGGGGSEVKLEESGAELVKP |
| | | GASVKLSCRTSGFN1KDTIHWVKQRPEQGLK |
| | | WIGRIDPANGNTKYDPKFQDKATVTADTSSN |
| | | TAYLQLSSLTSEDTAVYYCVTYAYDGNWYF |
| | | DVWGAGTAVTVSSPRASTTTPAPRPPTPAPTI |
| | | ASQPLSLRPEACRPAAGGAVHTRGLDFACDF |
| | | WVLVVVGGVLACYSLLVTVAFIIFWVRSKRS |
| | | RLLHSDYMNMTPRRPGPTRKHYQPYAPPRDF |
| | | AAYRSRVKFSRSADAPAYKQGQNQLYNELN |
| | | LGRREEYDVLDKRRGRDPEMGGKPRRKNPQ |
| | | EGLYNELQKDKMAEAYSEIGMKGERRRGKG |
| | | HDGLYQGLSTATKDTYDALHMQALPPRRTD |
| | | GSGATNFSLLKQAGDVEENPGPVSEAMPRG |
| | | WTALCLLSLLPSGFMSLDNNGTATPELPTQG |
| | | TFSNVSTNVSYQETTTPSTLGSTSLHPVSQHG |
| | | NEATTNITETTVKFTSTSVITSVYGNTNSSVQS |
| | | QTSVISTVFTTPANVSTPETTLKPSLSPGNVSD |
| | | LSTTSTSLATSPTKPYTSSSPILSDIKAEIKCSGI |
| | | REVKLTQGICLEQNKTSSCAEFKKDRGEGLA |
| | | RVLCGEEQADADAGAQVCSLLLAQSEVRPQ |
| | | CLLLVLANRTEISSKLQLMKKHQSDLKKLGIL |
| | | DFTEQDVASHQSYSQKTLIALVTSGALLAVL |
| | | GITGYFLMNRRSWSPI |
| CD2 V_{L}- | SEQ ID | MALPVTALLLPLALLLHAARPDIKNITQSPSS |
| OKT3 V_{L}- | NO: 44 | MYVSLGERVTITCKASQDINSFLSWFQQKPG |
| OKT3 V_{H}- | | KSPKTLIYRANRLVDGVPSRFSGSGSGQDYSL |
| CD2 V_{H} | | TISSLEYEDMEIYYCLQYDEFPYTFGGGTKLE |
| | | MKRGGGGSGGGGSGGGGSGGGGSQIVLTQS |
| | | PAIMSASPGEKVTMTCSASSSVSYMNWYQQ |
| | | KSGTSPKRWIYDTSKLASGVPAHFRGSGSGTS |
| | | YSLTISGMEAEDAATYYCQQWSSNPFTFGSG |
| | | TKLEINRGGGGSGGGGSGGGGSGGGGSGGG |
| | | GSGGGGSGGGGSGGGGSGGGGSGGGGSGSQ |
| | | VQLQQSGAELARPGASVKMSCKASGYTFTR |
| | | YTMHWVKQRPGQGLEWIGYINPSRGYTNYN |
| | | QKFKDKATLTTDKSSSTAYMQLSSLTSEDSA |
| | | VYYCARYYDDHYCLDYWGQGTTLTVSSGG |
| | | GGSGGGGSGGGGSGGGGSEVKLEESGAELV |
| | | KPGASVKLSCRTSGFN1KDTIHWVKQRPEQGL |
| | | KWIGRIDPANGNTKYDPKFQDKATVTADTSS |
| | | NTAYLQLSSLTSEDTAVYYCVTYAYDGNWY |
| | | FDVWGAGTAVTVSSPRASTTTPAPRPPTPAPT |
| | | IASQPLSLRPEACRPAAGGAVHTRGLDFACDF |
| | | WVLVVVGGVLACYSLLVTVAFIIFWVRSKRS |
| | | RLLHSDYMNMTPRRPGPTRKHYQPYAPPRDF |
| | | AAYRSRVKFSRSADAPAYKQGQNQLYNELN |
| | | LGRREEYDVLDKRRGRDPEMGGKPRRKNPQ |
| | | EGLYNELQKDKMAEAYSEIGMKGERRRGKG |
| | | HDGLYQGLSTATKDTYDALHMQALPPRRTD |
| | | GSGATNFSLLKQAGDVEENPGPVSEAMPRG |
| | | WTALCLLSLLPSGFMSLDNNGTATPELPTQG |
| | | TFSNVSTNVSYQETTTPSTLGSTSLHPVSQHG |
| | | NEATTNITETTVKFTSTSVITSVYGNTNSSVQS |
| | | QTSVISTVFTTPANVSTPETTLKPSLSPGNVSD |
| | | LSTTSTSLATSPTKPYTSSSPILSDIKAEIKCSGI |
| | | REVKLTQGICLEQNKTSSCAEFKKDRGEGLA |
| | | RVLCGEEQADADAGAQVCSLLLAQSEVRPQ |
| | | CLLLVLANRTEISSKLQLMKKHQSDLKKLGIL |
| | | DFTEQDVASHQSYSQKTLIALVTSGALLAVL |
| | | GITGYFLMNRRSWSPI |
| CD7 V_{L}- | SEQ ID | MALPVTALLLPLALLLHAARPDIQMTQTTSSL |
| CD2 V_{L}- | NO: 45 | SASLGDRVTISCSASQGISNYLNWYQQKPDG |
| CD2 V_{H}- | | TVKLLIYYTSSLHSGVPSRFSGSGSGTDYSLTI |
| CD7 V_{H} | | SNLEPEDIATYYCQQYSKLPYTFGGGTKLEIK |
| | | RGGGGSGGGGSGGGGSGGGGSDIKNITQSPS |
| | | SMYVSLGERVTITCKASQDINSFLSWFQQKPG |
| | | KSPKTLIYRANRLVDGVPSRFSGSGSGQDYSL |
| | | TISSLEYEDMEIYYCLQYDEFPYTFGGGTKLE |
| | | MKRGGGGSGGGGSGGGGSGGGGSGGGGSG |
| | | GGGSGGGGSGGGGSGGGGSGGGGSEVKLEE |
| | | SGAELVKPGASVKLSCRTSGFN1KDTIHWVK |
| | | QRPEQGLKWIGRIDPANGNTKYDPKFQDKAT |
| | | VTADTSSNTAYLQLSSLTSEDTAVYYCVTYA |
| | | YDGNWYFDVWGAGTAVTVSSGGGGSGGGG |
| | | SGGGGSGGGGSEVQLVESGGGLVKPGGSLKL |
| | | SCAASGLTFSSYAMSWVRQTPEKRLEWVASI |
| | | SSGGFTYYPDSVKGRFTISRDNARNILYLQMS |
| | | SLRSEDTAMYYCARDEVRGYLDVWGAGTTV |
| | | TVSPRASTTTPAPRPPTPAPTIASQPLSLRPEAC |
| | | RPAAGGAVHTRGLDFACDFWVLVVVGGVLA |
| | | CYSLLVTVAFIIFWVRSKRSRLLHSDYMNMT |
| | | PRRPGPTRKHYQPYAPPRDFAAYRSRVKFSRS |
| | | ADAPAYKQGQNQLYNELNLGRREEYDVLDK |
| | | RRGRDPEMGGKPRRKNPQEGLYNELQKDKM |
| | | AEAYSEIGMKGERRRGKGHDGLYQGLSTAT |
| | | KDTYDALHMQALPPRRTDGSGATNFSLLKQ |
| | | AGDVEENPGPVSEAMPRGWTALCLLSLLPSG |
| | | FMSLDNNGTATPELPTQGTFSNVSTNVSYQE |
| | | TTTPSTLGSTSLHPVSQHGNEATTNITETTVKF |
| | | TSTSVITSVYGNTNSSVQSQTSVISTVFTTPAN |
| | | VSTPETTLKPSLSPGNVSDLSTTSTSLATSPTK |
| | | PYTSSSPILSDIKAEIKCSGIREVKLTQGICLEQ |
| | | NKTSSCAEFKKDRGEGLARVLCGEEQADAD |
| | | AGAQVCSLLLAQSEVRPQCLLLVLANRTEISS |
| | | KLQLMKKHQSDLKKLGILDFTEQDVASHQSY |
| | | SQKTLIALVTSGALLAVLGITGYFLMNRRSWSPI |
| CD2 V_{L}- | SEQ ID | MALPVTALLLPLALLLHAARPDIKNITQSPSS |
| CD7 V_{L}- | NO: 46 | MYVSLGERVTITCKASQDINSFLSWFQQKPG |
| CD7 V_{H}- | | KSPKTLIYRANRLVDGVPSRFSGSGSGQDYSL |
| CD2 V_{H} | | TISSLEYEDMEIYYCLQYDEFPYTFGGGTKLE |
| | | MKRGGGGSGGGGSGGGGSGGGGSDIQMTQT |
| | | TSSLSASLGDRVTISCSASQGISNYLNWYQQK |
| | | PDGTVKLLIYYTSSLHSGVPSRFSGSGSGTDY |
| | | SLTISNLEPEDIATYYCQQYSKLPYTFGGGTK |
| | | LEIKRGGGGSGGGGSGGGGSGGGGSGGGGS |
| | | GGGGSGGGGSGGGGSGGGGSGGGGSEVQLV |
| | | ESGGGLVKPGGSLKLSCAASGLTFSSYAMSW |
| | | VRQTPEKRLEWVASISSGGFTYYPDSVKGRFT |
| | | ISRDNARNILYLQMSSLRSEDTAMYYCARDE |
| | | VRGYLDVWGAGTTVTVSGGGGSGGGGSGG |
| | | GGSGGGGSEVKLEESGAELVKPGASVKLSCR |
| | | TSGFN1KDTIHWVKQRPEQGLKWIGRIDPANG |
| | | NTKYDPKFQDKATVTADTSSNTAYLQLSSLT |
| | | SEDTAVYYCVTYAYDGNWYFDVWGAGTAV |
| | | TVSSPRASTTTPAPRPPTPAPTIASQPLSLRPEA |
| | | CRPAAGGAVHTRGLDFACDFWVLVVVGGVL |
| | | ACYSLLVTVAFIIFWVRSKRSRLLHSDYMNM |
| | | TPRRPGPTRKHYQPYAPPRDFAAYRSRVKFS |
| | | RSADAPAYKQGQNQLYNELNLGRREEYDVL |
| | | DKRRGRDPEMGGKPRRKNPQEGLYNELQKD |
| | | KMAEAYSEIGMKGERRRGKGHDGLYQGLST |
| | | ATKDTYDALHMQALPPRRTDGSGATNFSLLK |
| | | QAGDVEENPGPVSEAMPRGWTALCLLSLLPS |
| | | GFMSLDNNGTATPELPTQGTFSNVSTNVSYQ |
| | | ETTTPSTLGSTSLHPVSQHGNEATTNITETTVK |
| | | FTSTSVITSVYGNTNSSVQSQTSVISTVFTTPA |
| | | NVSTPETTLKPSLSPGNVSDLSTTSTSLATSPT |
| | | KPYTSSSPILSDIKAEIKCSGIREVKLTQGICLE |
| | | QNKTSSCAEFKKDRGEGLARVLCGEEQADA |
| | | DAGAQVCSLLLAQSEVRPQCLLLVLANRTEIS |
| | | SKLQLMKKHQSDLKKLGILDFTEQDVASHQS |
| | | YSQKTLIALVTSGALLAVLGITGYFLMNRRSWSPI |

Additionally, provided herein are hairpin tandem CAR constructs which may incorporate V_{H} and V_{L} domains of scFvs targeting any of the antigen pairs provided in Table 5.

TABLE 8
Hairpin Tandem CAR Constructs

| 8-I | 8-II | 8-III | 8-IV | 8-V | 8-VI | 8-VII | 8-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₃₁₀₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 8-IX | 8-X | 8-XI | 8-XII | 8-XIII | 8-XIV | 8-XV | 8-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ | (SEQ | (SEQ | (SEQ | (SEQ | (SEQ | (SEQ |
| NO: 449) | ID | ID | ID | ID | ID | ID | ID |
| | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 8-XVII | 8-XVIII | 8-XIX | 8-XX | 8-XXI | 8-XXII | 8-XXIII | 8-XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ | (SEQ | (SEQ | (SEQ | (SEQ | (SEQ | (SEQ |
| NO: 449) | ID | ID | ID | ID | ID | ID | ID |
| | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 8-XXV | 8-XXVI | 8-XXVII | 8-XXVIII | 8-XIX | 8-XXX | 8-XXXI | 8-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ | (SEQ | (SEQ | (SEQ | (SEQ | (SEQ | (SEQ |
| NO: 447) | ID | ID | ID | ID | ID | ID | ID |
| | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ | (SEQ | (SEQ | (SEQ | (SEQ | (SEQ | (SEQ |
| NO: 449) | ID | ID | ID | ID | ID | ID | ID |
| | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |

For example, provided herein in Table 9 are hairpin tandem CAR constructs which incorporate the V_{H} and V_{L} domains of CD2 and CD3 scFvs.

TABLE 9
Hairpin Tandem CAR Constructs Targeting CD2 and CD3

| Example 1 | Example 2 | Example 3 | Example 4 | Example 5 | Example 6 | Example 7 | Example 8 |
|---|---|---|---|---|---|---|---|
| CD8a | CD8a | CD8a | CD8a | CD8a | CD8a | CD8a | CD8a |
| CD3-V_{L} | CD3-V_{L} | CD3-V_{L} | CD3-V_{L} | CD2-V_{L} | CD2-V_{L} | CD3-V_{L} | CD3-V_{L} |
| GGGGS₄ | GGGGS₄ | GGGGS₄ | GGGGS₄ | GGGGS₄ | GGGGS₄ | GGGGS₄ | GGGGS4 |
| (SEQ ID | (SEQ ID | (SEQ ID | (SEQ ID | (SEQ ID | (SEQ ID | (SEQ ID | (SEQ ID |
| NO: 450) | NO: 450) | NO: 450) | NO: 450) | NO: 450) | NO: 450) | NO: 450) | NO: 450) |
| CD2-V_{L} | CD2-V_{L} | CD2-V_{L} | CD2-V_{L} | CD3-V_{L} | CD3-V_{L} | CD2-V_{L} | CD2-V_{L} |
| (GGGGS)₁₀ | GGGGS | (GGGGS)₁₀ | GGGGS | (GGGGS)₁₀ | GGGGS | (GGGGS)₁₀ | GGGGS |
| (SEQ | GGGGC | (SEQ | GGGGC | (SEQ | GGGGC | (SEQ | GGGGC |
| ID | (GGGGS)₂ | ID | (GGGGS)₂ | ID | (GGGGS)₂ | ID | (GGGGS)₂ |
| NO: 451) | GGGGP | NO: 451) | GGGGP | NO: 451) | GGGGP | NO: 451) | GGGGP |
| | (GGGGS)₃ | | (GGGGS)₃ | | (GGGGS)₃ | | (GGGGS)₃ |
| | GGGG | | GGGG | | GGGG | | GGGG |
| | CGGGG | | CGGGG | | CGGGG | | CGGGG |
| | S (SEQ | | S (SEQ | | S (SEQ | | S (SEQ |
| | ID | | ID | | ID | | ID |
| | NO: 448) | | NO: 448) | | NO: 448) | | NO: 448) |
| CD2-V_{H} | CD2-V_{H} | CD2-V_{H} | CD2-V_{H} | CD3-V_{H} | CD3-V_{H} | CD2-V_{H} | CD2-V_{H} |
| GGGGS₄ | GGGGS₄ | GGGGS₄ | GGGGS₄ | GGGGS₄ | GGGGS₄ | GGGGS₄ | GGGGS₄ |
| CD3-V_{H} | CD3-V_{H} | CD3-V_{H} | CD3-V_{H} | CD2-V_{H} | CD2-V_{H} | CD3-V_{H} | CD3-V_{H} |
| CD28 Tm | CD28 Tm | CD28 Tm | CD28 Tm | CD28 Tm | CD28 Tm | CD28 Tm | CD28 Tm |
| CD28 | CD28 | CD28 | CD28 | CD28 | CD28 | CD28 | CD28 |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| P2A | P2A | P2A | P2A | P2A | P2A | P2A | P2A |
| CD34 | CD34 | CD34 | CD34 | CD34 | CD34 | CD34 | CD34 |

Also provided herein in Table 10 are hairpin tandem CAR constructs with a (Cys=Cys) double-stranded bond (DSB) which may incorporate the VH and VL domains of scFvs targeting any of the antigen pairs provided in Table 5.

TABLE 10
Hairpin Tandem DSB CAR Constructs with a (Cys = Cys) Double-Stranded Bond
(DSB).

| 10-I | 10-II | 10-III | 10-IV | 10-V | 10-VI | 10-VII | 10-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 10-IX | 10-X | 10-XI | 10-XII | 10-XIII | 10-XIV | 10-XV | 10-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| 10-XVII | 10-XVIII | 10-XIX | 10-XX | 10-XXI | 10-XXII | 10-XXIII | 10-XXIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| | | | 10- | | | | |
| 10-XXV | 10-XXVI | 10-XXVII | XXVIII | 10-XXIX | 10-XXX | 10-XXXI | 10-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ SEQ | SEQ | SEQ | SEQ | SEQ | SEQ | SEQ | |
| ID | ID | ID | ID | ID | ID | ID | ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| SC₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ SEQ | S₍₀₋₁₎ SEQ | S₍₀₋₁₎ SEQ | S₍₀₋₁₎ SEQ | S₍₀₋₁₎ SEQ | S₍₀₋₁₎ SEQ | S₍₀₋₁₎ SEQ | S₍₀₋₁₎ SEQ |
| ID | ID | ID | ID | ID | ID | ID | ID |
| NO: 448) | NO: 448) | NO: 448) | NO: 448) | NO: 448) | NO: 448) | NO: 448) | NO: 448) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| SEQ | SEQ | SEQ | SEQ | SEQ | SEQ | SEQ | SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |

Dual iNKT-CAR Cells

In certain embodiments, the disclosure provides an engineered iNKT cell comprising a dual CAR (dCAR), i.e., two CARs expressed from a single lentivirus construct, that specifically binds CD7 and CD2, wherein the iNKT cell is deficient in CD7 and CD2 (e.g., CD7×CD2-iNKT-dCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-dCARΔCD7ΔCD2 cells encodes a modified Human-Herpes Simplex Virus-1-thymidine kinase (TK) gene fused in-frame to the extracellular and transmembrane domains of the human CD34 cDNA.

In a similar manner, other dual iNKT-CARs may be constructed, and are given below in Table 11.

TABLE 11
Dual CARs and inKTs.

| | Antigen Target | Antigen Deletion/ |
|---|---|---|
| Example | of iNKT-CAR | Suppression |
| D1 | CD2 × CD3ε | — |
| D2 | CD2 × CD3ε | CD2 |
| D3 | CD2 × CD3ε | CD3ε |
| D4 | CD2 × CD3ε | CD2 and CD3ε |
| D5 | CD2 × CD4 | — |
| D6 | CD2 × CD4 | CD2 |
| D7 | CD2 × CD4 | CD4 |
| D8 | CD2 × CD4 | CD2 and CD4 |
| D9 | CD2 × CD5 | — |
| D10 | CD2 × CD5 | CD2 |
| D11 | CD2 × CD5 | CD5 |
| D12 | CD2 × CD5 | CD2 and CD5 |
| D13 | CD2 × CD7 | — |
| D14 | CD2 × CD7 | CD2 |
| D15 | CD2 × CD7 | CD7 |
| D16 | CD2 × CD7 | CD2 and CD7 |
| D17 | CD3ε × CD4 | — |
| D18 | CD3ε × CD4 | CD3ε |
| D19 | CD3ε × CD4 | CD4 |
| D20 | CD3ε × CD4 | CD3ε and CD4 |
| D21 | CD3ε × CD5 | — |
| D22 | CD3ε × CD5 | CD3ε |
| D23 | CD3ε × CD5 | CD5 |
| D24 | CD3ε × CD5 | CD3ε and CD5 |
| D25 | CD3ε × CD7 | — |
| D26 | CD3ε × CD7 | CD3ε |
| D27 | CD3ε × CD7 | CD7 |
| D28 | CD3ε × CD7 | CD3ε and CD7 |
| D29 | CD4 × CD5 | — |
| D30 | CD4 × CD5 | CD4 |
| D31 | CD4 × CD5 | CD5 |
| D32 | CD4 × CD5 | CD4 and CD5 |
| D33 | CD4 × CD7 | — |
| D34 | CD4 × CD7 | CD4 |
| D35 | CD4 × CD7 | CD7 |
| D36 | CD4 × CD7 | CD5 and CD7 |
| D37 | CD5 × CD7 | — |
| D38 | CD5 × CD7 | CD5 |
| D39 | CD5 × CD7 | CD7 |
| D40 | CD5 × CD7 | CD5 and CD7 |
| D41 | TRAC × CD2 | — |
| D42 | TRAC × CD2 | TRAC |
| D43 | TRAC × CD2 | CD2 |
| D44 | TRAC × CD2 | TRAC and CD2 |
| D45 | TRAC × CD3ε | — |
| D46 | TRAC × CD3ε | TRAC |
| D47 | TRAC × CD3ε | CD3ε |
| D48 | TRAC × CD3ε | TRAC and CD3ε |
| D49 | TRAC × CD4 | — |
| D50 | TRAC × CD4 | TRAC |
| D51 | TRAC × CD4 | CD4 |
| D52 | TRAC × CD4 | TRAC and CD4 |
| D53 | TRAC × CD5 | — |
| D54 | TRAC × CD5 | TRAC |
| D55 | TRAC × CD5 | CD5 |
| D56 | TRAC × CD5 | TRAC and CD5 |
| D57 | TRAC × CD7 | — |
| D58 | TRAC × CD7 | TRAC |
| D59 | TRAC × CD7 | CD7 |
| D60 | TRAC × CD7 | TRAC and CD7 |
| D61 | TCRβ × CD2 | — |
| D62 | TCRβ × CD2 | TCRβ |
| D63 | TCRβ × CD2 | CD2 |
| D64 | TCRβ × CD2 | TCRβ and CD2 |
| D65 | TCRβ × CD3ε | — |
| D66 | TCRβ × CD3ε | TCRβ |
| D67 | TCRβ × CD3ε | CD3ε |
| D68 | TCRβ × CD3ε | TCRβ and CD3ε |
| D69 | TCRβ × CD4 | — |
| D70 | TCRβ × CD4 | TCRβ |
| D71 | TCRβ × CD4 | CD4 |
| D72 | TCRβ × CD4 | TCRβ and CD4 |
| D73 | TCRβ × CD5 | — |
| D74 | TCRβ × CD5 | TCRβ |
| D75 | TCRβ × CD5 | CD5 |
| D76 | TCRβ × CD5 | TCRβ and CD5 |
| D77 | TCRβ × CD7 | — |
| D78 | TCRβ × CD7 | TCRβ |
| D79 | TCRβ × CD7 | CD7 |
| D80 | TCRβ × CD7 | TCRβ and CD7 |
| D81 | BCMA × CS1 | — |
| D82 | BCMA × CS1 | CS1 |
| D83 | BCMA × CD19 | — |
| D84 | BCMA × CD38 | — |
| D85 | BCMA × CD38 | CD38 |
| D86 | CS1 × CD19 | — |
| D87 | CS1 × CD19 | CS1 |
| D88 | CS1 × CD38 | — |
| D89 | CS1 × CD38 | CS1 |
| D90 | CS1 × CD38 | CD38 |
| D91 | CS1 × CD38 | CS1 and CD38 |
| D92 | CD19 × CD38 | — |
| D93 | CD19 × CD38 | CD38 |
| D94 | APRIL × CS1 | — |
| D95 | APRIL × CS1 | CS1 |
| D96 | APRIL × BCMA | — |
| D97 | APRIL × CD19 | — |
| D98 | APRIL × CD38 | — |
| D99 | APRIL × CD38 | CD38 |

In a further aspect, an iNKT-CAR cell to be used as a control in certain circumstances may be created. For example, when designing iNKT-CARs binding T-cell antigens, the control iNKT-CAR may include an extracellular domain that binds to an antigen not expressed on a malignant T-cell. The antigen that the control iNKT-CAR cell binds to may be, e.g., CD19. CD19 is an antigen expressed on B cells but not on T cells, so an iNKT-CAR with an extracellular domain adapted to bind to CD19 will not bind to T cells. These iNKT-CARs may be called iNKT-CAR19 cells. These control iNKT-CAR cells may be used as controls to analyze the binding efficiencies and non-specific binding of iNKT-CAR cells targeted to the cancer of interest and/or recognizing the antigen of interest.

CARs may be further designed as disclosed in WO2018027036A1, optionally employing variations which will be known to those of skill in the art. Lentiviral vectors and cell lines can be obtained, and guide RNAs designed, validated, and synthesized, as disclosed therein as well as by methods known in the art and from commercial sources.

Engineered CARs may be introduced into iNKT cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1 0, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer RF and Saunders DN, “Experimental design for stable genetic manipulation in mammalian cell lines: lentivirus and alternatives,” Genes Cells 2015 January; 20(1):1-10.

### Cytokine Gene Deletion or Suppression

In addition to gene-editing the TCR and cell surface proteins and antigens, genes for secretable proteins such as cytokines and chemokines may be edited. Such editing would be done, e.g., to reduce or prevent the development or maintenance of cytokine release syndrome (CRS). CRS is caused by a large, rapid release of cytokines from immune cells in response to immunotherapy (or other immunological stimulus). Modifying, disrupting, or deleting one or more cytokine or chemokine genes can be accomplished using the methods known in the art, such as genetic ablation (gene silencing) in which gene expression is abolished through the alteration or deletion of genetic sequence information. This can be accomplished using known genetic engineering tools in the art, such as Transcription Activator-like Effector Nucleases (TALENs), Zinc Finger Nucleases (ZFNs), CRISPR, by transduction of small hairpin RNAs (shRNAs), by targeted transduction of a CAR into the gene sequence of the cytokine, and the like.

Cytokines or chemokines that can be deleted from immune effector cells as disclosed herein, e.g., using Cas9-CRISPR or by targeted transduction of a CAR into the gene sequence of the cytokine, include without limitation the following: XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, MSP, A2M, ACKR1, ACKR2, ACKR3, ACVR1, ACVR2B, ACVRL1, ADIPOQ, AGER, AGRN, AIMP1, AREG, BMP1, BMP10, BMP15, BMP2, BMP3, BMP4, BMP5, BMP6, BMP7, BMP8A, BMP8B, BMPR2, C10orf99, C1QTNF4, C5, CCL28, CCR1, CCR2, CCR3, CCR5, CCR6, CCR7, CD109, CD36, CD4, CD40LG, CD74, CER1, CHRD, CKLF, CLCF1, CMTM1, CMTM2, CMTM3, CMTM4, CMTM5, CMTM6, CMTM7, CMTM8, CNTF, CNTFR, COPS5, CRLF1, CSF1, CSF1R, CSF2, CSF3, CSF3R, CTF1, CX3CR1, CXCL16, CXCL17, CXCR1, CXCR2, CXCR3, CXCR4, CXCR6, EBI3, EDN1, ELANE, ENG, FAM3B, FAM3C, FAM3D, FAS, FASLG, FGF2, FLT3LG, FZD4, GBP1, GDF1, GDF10, GDF11, GDF15, GDF2, GDF3, GDF5, GDF6, GDF7, GDF9, GPI, GREM1, GREM2, GRN, HAX1, HFE2, HMGB1, HYAL2, IFNA10, IFNA14, IFNA16, IFNA2, IFNA5, IFNA6, IFNA8, IFNAR1, IFNAR2, IFNB1, IFNE, IFNG, IFNGR1, IFNK, IFNL1, IFNL3, IFNW1, IL10RA, IL11RA, IL12A, IL12B, IL12RB1, IL17A, IL17B, IL17C, IL17D, IL17F, IL18BP, IL-19, IL1F10, IL1R1, IL1R2, IL1RAPL1, IL1RL1, IL1RN, IL20RA, IL20RB, IL21, IL22, IL22RA1, IL22RA2, IL23A, IL23R, IL24, IL25, IL26, IL27, IL2RA, IL2RB, IL2RG, IL31, IL31RA, IL32, IL33, IL34, IL36A, IL36B, IL36G, IL36RN, IL37, IL6R, IL6ST, INHA, INHBA, INHBB, INHBC, INHBE, ITGA4, ITGAV, ITGB1, ITGB3, KIT, KITLG, KLHL20, LEFTY1, LEFTY2, LIFR, LTA, LTB, LTBP1, LTBP3, LTBP4, MIF, MINOS1-, MSTN, NAMPT, NBL1, NDP, NLRP7, NODAL, NOG, NRG1, NRP1, NRP2, OSMR, PARK7, PDPN, PF4, PF4V1, PGLYRP1, PLP2, PPBP, PXDN, SCG2, SCGB3A1, SECTM1, SLURP1, SOSTDC1, SP100, SPP1, TCAP, TGFBR1, TGFBR2, TGFBR3, THBS1, THNSL2, THPO, TIMP1, TNF, TNFRSF11, TNFRSF1A, TNFRSF9, TNFRSF10, TNFSF11, TNFSF12, TNFSF12-, TNFSF13, TNFSF13B, TNFSF14, TNFSF15, TNFSF18, TNFSF4, TNFSF8, TNFSF9, TRIM16, TSLP, TWSG1, TXLNA, VASN, VEGFA, VSTM1, WFIKKN1, WFIKKN2, WNT1, WNT2, WNT5A, WNT7A, and ZFP36.

The sequences of these genes are known and available in the art.

Indications and Standards of Care in ACT (iNKT) Therapy

In some embodiment, the genome-edited immune effector cells disclosed herein, and/or generated using the methods disclosed herein, express one or more chimeric antigen receptors (CARs) and can be used as a medicament, i.e., for the treatment of disease. In many embodiments, the cells are iNKT cells.

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin's lymphoma, and T-cell chronic lymphocytic leukemia (T-CLL).

In some embodiments, the cells may be used in the treatment of autoimmune diseases such as lupus, autoimmune (rheumatoid) arthritis, multiple sclerosis, transplant rejection, Crohn's disease, ulcerative colitis, dermatitis, and the like. In some embodiments, the cells are chimeric autoantibody receptor T-cells, or iNKT displaying antigens or fragments thereof, instead of antibody fragments; in this version of adoptive cell transfer, the B cells that cause autoimmune diseases will attempt to attack the engineered T cells, which will respond by killing them.

In some embodiments, the cells may be used in the treatment of infectious diseases such as HIV and tuberculosis.

In another embodiment, the iNKT cells of the present disclosure can undergo robust in vivo T cell expansion and can persist for an extended amount of time.

In some embodiments, the treatment of a patient with iNKT cells of the present disclosure can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic, is meant that the cells or population of cells used for treating patients are not originating from the patient but from a donor.

The treatment of cancer with iNKT cells of the present disclosure may be in combination with one or more therapies selected from antibody therapy, chemotherapy, cytokine therapy, dendritic cell therapy, gene therapy, hormone therapy, radiotherapy, laser light therapy, and radiation therapy.

The administration of iNKT cells or a population of iNKT cells of the present disclosure of the present disclosure be carried out by aerosol inhalation, injection, ingestion, transfusion, implantation or transplantation. The iNKT cells compositions described herein, i.e., mono CAR, dual CAR, tandem CARs, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

The administration of iNKT cells or a population of iNKT cells can consist of the administration of 10⁴-10⁹ cells per kg body weight, preferably 10⁵ to 10⁶ cells/kg body weight including all integer values of cell numbers within those ranges. The iNKT cells or a population of iNKT cells can be administrated in one or more doses. In another embodiment, the effective amount of iNKT cells or a population of iNKT cells are administrated as a single dose. In another embodiment, the effective amount of cells are administered as more than one dose over a period time. Timing of administration is within the judgment of a health care provider and depends on the clinical condition of the patient. The iNKT cells or a population of iNKT cells may be obtained from any source, such as a blood bank or a donor. While the needs of a patient vary, determination of optimal ranges of effective amounts of a given iNKT cell population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of iNKT cells or a population of iNKT cells or composition comprising those iNKT cells are administered parenterally. The administration can be an intravenous administration. The administration of iNKT cells or a population of iNKT cells or composition comprising those iNKT cells can be directly done by injection within a tumor.

In one embodiment of the present disclosure, the iNKT cells or a population of the iNKT cells are administered to a patient in conjunction with, e.g., before, simultaneously or following, any number of relevant treatment modalities, including but not limited to, treatment with cytokines, or expression of cytokines from within the iNKT cells, that enhance iNKT cell proliferation and persistence and, include but not limited to, IL-2, IL-7, and IL-15 or analogues thereof.

In some embodiments, the iNKT cells or a population of iNKT cells of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the iNKT cells or a population of iNKT cells of the present disclosure may be used in combination with T-cell checkpoint inhibitors, including but not limited to, anti-CTLA4 (Ipilimumab) anti-PD1 (Pembrolizumab, Nivolumab, Cemiplimab), anti-PDL1 (Atezolizumab, Avelumab, Durvalumab), anti-PDL2, anti-BTLA, anti-LAG3, anti-TIM3, anti-VISTA, anti-TIGIT, and anti-MR.

In another embodiment, the iNKT cells or a population of iNKT cells of the present disclosure may be used in combination with T cell agonists, including but not limited to, antibodies that stimulate CD28, ICOS, OX-40, CD27, 4-1BB, CD137, GITR, and HVEM.

In another embodiment, the iNKT cells or a population of iNKT cells of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, iNKT cells may be co-administered with α-GalCer and IL-12, as both of these compounds work synergistically for iNKT activation.

In another embodiment, the iNKT cells or a population of iNKT cells of the present disclosure may be used in combination with immunostimulatory therapies, such as toll-like receptors agonists, including but not limited to, TLR3, TLR4, TLR7 and TLR9 agonists.

In another embodiment, the iNKT cells or a population of iNKT cells of the present disclosure may be used in combination with stimulator of interferon gene (STING) agonists, such as cyclic GMP-AMP synthase (cGAS).

Immune effector cell aplasia, particularly T cell aplasia is also a concern after adoptive cell transfer therapy. When the malignancy treated is a T-cell malignancy, and iNKT cells target a T cell antigen, normal T cells and their precursors expressing the antigen will become depleted, and the immune system will be compromised. Accordingly, methods for managing these side effects are attendant to therapy. Such methods include selecting and retaining non-malignant T cells or precursors, either autologous or allogeneic (optionally engineered not to cause rejection or be rejected), for later expansion and re-infusion into the patient, after iNKT cells are exhausted or deactivated. Alternatively, iNKT cells which recognize and kill subsets of TCR-bearing cells, such as normal and malignant TRBC1⁺, but not TRBC2⁺ cells, or alternatively, TRBC2⁺, but not TRBC1⁺ cells, may be used to eradicate a T cell malignancy while preserving sufficient normal T cells to maintain normal immune system function.

### Definitions

Unless specifically defined herein, all technical and scientific terms used have the same meaning as commonly understood by a skilled artisan in the fields of gene therapy, biochemistry, genetics, and molecular biology. All disclosed compositions and methods similar or equivalent to those described herein can be used in the practice or testing of the present disclosure.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “activation” (and other conjugations thereof) in reference to cells is generally understood to be synonymous with “stimulating” and as used herein refers to treatment of cells that results in expansion of cell populations. In T cells, activation is often accomplished by exposure to CD2 and CD28 (and sometimes CD2 as well) agonists, typically antibodies, optionally coated onto magnetic beads or conjugated to a colloidal polymeric matrix.

The term “antigen” as used herein is a cell surface protein recognized by (i.e., that is the target of) T cell receptor or chimeric antigen receptor. In the classical sense antigens are substances, typically proteins, that are recognized by antibodies, but the definitions overlap insofar as the CAR comprises antibody-derived domains such as light (VL) and heavy (VH) chains recognizing one or more antigen(s).

The term “cancer” refers to a malignancy or abnormal growth of cells in the body. Many different cancers can be characterized or identified by particular cell surface proteins or molecules. Thus, in general terms, cancer in accordance with the present disclosure may refer to any malignancy that may be treated with an immune effector cell, such as a iNKT cell as described herein, in which the immune effector cell recognizes and binds to the cell surface protein on the cancer cell. As used herein, cancer may refer to a hematologic malignancy, such as multiple myeloma, a T-cell malignancy, or a B cell malignancy. T cell malignancies may include, but are not limited to, T-cell acute lymphoblastic leukemia (T-ALL) or non-Hodgkin's lymphoma. A cancer may also refer to a solid tumor, such as including, but not limited to, cervical cancer, pancreatic cancer, ovarian cancer, mesothelioma, and lung cancer.

A “cell surface protein” as used herein is a protein (or protein complex) expressed by a cell at least in part on the surface of the cell. Examples of cell surface proteins include the TCR (and subunits thereof) and CD7.

A “chimeric antigen receptor” or “CAR” as used herein and generally used in the art, refers to a recombinant fusion protein that has an extracellular ligand-binding domain, a transmembrane domain, and a signaling transducing domain that directs the cell to perform a specialized function upon binding of the extracellular ligand-binding domain to a component present on the target cell. For example, a CAR can have an antibody-based specificity for a desired antigen (e.g., tumor antigen) with a T cell receptor-activating intracellular domain to generate a chimeric protein that exhibits specific anti-target cellular immune activity. First-generation CARs include an extracellular ligand-binding domain and signaling transducing domain, commonly CD3ζ or FcεRIγ. Second generation CARs are built upon first generation CAR constructs by including an intracellular costimulatory domain, commonly 4-1BB or CD28. These costimulatory domains help enhance iNKT cell cytotoxicity and proliferation compared to first generation CARs. The third generation CARs include multiple costimulatory domains, primarily to increase iNKT cell proliferation and persistence. Chimeric antigen receptors are distinguished from other antigen binding agents by their ability both to bind MHC-independent antigens and transduce activation signals via their intracellular domain.

A “CAR-bearing immune effector cell” is an immune effector cell which has been transduced with at least one CAR. A “CAR-iNKT cell” is a iNKT cell which has been transduced with at least one CAR; CAR-iNKT cells can be mono, dual, or tandem CAR-iNKT cells. CAR-iNKT cells can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

The term CAR-iNKT cell (equivalently, iNKT-CAR) means an iNKT cell that expresses a chimeric antigen receptor.

A dual iNKT-CAR cell (equivalently, iNKT-dCAR) is an iNKT-CAR cell that expresses two distinct chimeric antigen receptor polypeptides with affinity to different target antigens expressed within the same effector cell, wherein each CAR functions independently. The car may be expressed from a single or multiple polynucleotide sequences.

A tandem iNKT-CAR cell (equivalently, iNKT-tCAR) is an iNKT-CAR cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” as used herein refers to an immunotherapeutic cell population combination with one or more therapeutically acceptable carriers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “fratricide” as used herein means a process which occurs when an iNKT-CAR cell becomes the target of, and is killed by, another iNKT-CAR cell comprising the same chimeric antigen receptor as the target of iNKT-CAR cell, because the targeted cell expresses the antigen specifically recognized by the chimeric antigen receptor on both cells. iNKT-CARs comprising a chimeric antigen receptor which are deficient in an antigen to which the chimeric antigen receptor specifically binds will be “fratricide-resistant.”

The term “genome-edited” as used herein means having a gene added, deleted, or modified to be non-functional. Thus, in certain embodiments, a “gene-edited iNKT cell” is an iNKT cell that has had a gene such as a CAR recognizing at least one antigen added; and/or has had a gene such as the gene(s) to the antigen(s) that are recognized by the CAR deleted.

As used herein, “suicide gene” refers to a nucleic acid sequence introduced to a iNKT cell by standard methods known in the art, that when activated result in the death of the iNKT cell. If required suicide genes may facilitate the tracking and elimination, i.e., killing, of iNKT cells in vivo. Facilitated killing of iNKT cell cells by activating a suicide gene can be accomplished by standard methods known in the art. Suicide gene systems known in the art include, but are not limited to, include (a) herpes simplex virus (HSV)-tk which turns the nontoxic prodrug ganciclovir (GCV) into GCV-triphosphate, leading to cell death by halting DNA replication, (b) iCasp9 can bind to the small molecule AP1903 and result in dimerization, which activates the intrinsic apoptotic pathway, and (c) Targetable surface antigen expressed in the transduced iNKT cells (e.g., CD20 and truncated EGFR), allowing eliminating the modified cells efficiently through complement/antibody-dependent cellular cytotoxicity (CDC/ADCC) after administration of the associated monoclonal antibody.

A “cancer cell”, for example, is a malignant T cell, malignant B cell, or malignant plasma cell.

A “malignant B cell” is a B cell derived from a B-cell malignancy. B cell malignancies include, without limitation, (DLBCL), chronic lymphocytic leukemia (CLL)/small lymphocytic lymphoma (SLL), and B cell-precursor acute lymphoblastic leukemia (ALL).

A “malignant T cell” is a T cell derived from a T-cell malignancy.

The term “T-cell malignancy” refers to a broad, highly heterogeneous grouping of malignancies derived from T-cell precursors, mature T cells, or natural killer cells. Non-limiting examples of T-cell malignancies include T-cell acute lymphoblastic leukemia/lymphoma (T-ALL), human T-cell leukemia virus type 1-positive (HTLV-1+) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

A “healthy donor,” as used herein, is one who does not have a hematologic malignancy (e.g. a T-cell malignancy).

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “therapeutically effective” is intended to qualify the amount of active ingredients used in the treatment of a disease or disorder or on the effecting of a clinical endpoint.

As used herein, a “secretable protein” is s protein secreted by a cell which has an effect on other cells. By way of example, secretable proteins include ctyokines, chemokines, and transcription factors.

The term “donor template” refers to the reference genomic material that the cell uses as a template to repair the a double-stranded break through the homology-directed repair (HDR) DNA repair pathway. The donor template contains the piece of DNA to be inserted into the genome (containing the gene to be expressed, CAR, or marker) with two homology arms flanking the site of the double-stranded break. In some embodiments, a donor template may be an adeno-associated virus, a single-stranded DNA, or a double-stranded DNA.

The term “exposing to,” as used herein, in the context of bringing compositions of matter (such as antibodies) into intimate contact with other compositions of matter (such as cells), is intended to be synonymous with “incubated with,” and no lengthier period of time in contact is intended by the use of one term instead of the other.

The term “patient” is generally synonymous with the term “subject” and includes all mammals including humans.

The invention is further illustrated by the following examples.

### EXAMPLES

### Example 1—Method of Making Genome-Edited iNKT Cells

The following steps may be taken to provide the gene-edited iNKT cells disclosed herein. As those of skill in the art will recognize, certain of the steps may be conducted sequentially or out of the order listed below, though perhaps leading to different efficiency.

Step 1.

Peripheral blood mononuclear cells (PBMCs) are harvested from one or more healthy donors.

Step 2.

iNKT cells are then isolated/purified from a donor's PBMCs, for example using magnetic selection with a labelled antibody-coated magnetic beads that bind to Valpha24 (e.g., Miltenyi Biotec). Other purification techniques are known in the art and could be used.

Step 3.

iNKT cells are thereafter activated. There are several ways to activate iNKT. The non iNKT fraction remaining after purification may be irradiated (e.g. at 40Gy) and dosed with α-GalCer (e.g., 200 ng/ml for 1 hr at, e.g., 37° C. to generate cells that have CD1d-α-GalCer, the ligand for the invariant receptor). iNKT cells are then incubated with irradiated α-GalCer pulsed negative cells (1:10). Alternatively, an anti-iNKT receptor antibody could be used to activate iNKT. In yet another alternative, purified CD-1d complexed with α-GalCer could be used to activate iNKT. In yet another alternative CD1d expressing cell line pulsed with α-GalCer could be used to stimulate iNKT

Step 4.

If a CAR targeting one or more antigens is to be transduced into the cell, the antigen that is the target of the CAR may be deleted from the cell surface or its expression suppressed to prevent subsequent fratricide. Target deletion may be accomplished by electroporation with Cas9 mRNA and gRNA against the target(s). Other techniques, however, could be used to suppress expression of the target. These include other genome editing techniques such as TALENs, ZFNs, RNA interference, and eliciting of internal binding of the antigen to prevent cell surface expression. Deletion of the target may not be required in every circumstance. CD7, for instance is only expressed on 50% of iNKT, whereas CD2 is expressed on almost all iNKT. Examples of gRNAs that may be used include those shown in table 2, and others known in the art.

TABLE 12
Guide RNA sequences for use in removing surface antigens
on immune effector cells.

| Target | |
|---|---|
| gene | gRNA sequence |
| CD7 | 5′_2′OMe(A(ps)U(ps)C(ps))ACGGAGGUCAAUGUCUAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 47) |
| CD7g10 | 5′_2′OMe(G(ps)U(ps)A(ps))GACAUUGACCUCCGUGAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 48) |
| CD7g4 | 5′_2′OMe(A(ps)U(ps)C(ps))ACGGAGGUCAAUGUCUAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′(SEQ ID NO: 49) |
| TRACg | 5′_2′OMe(G(ps)A(ps)G(ps))AAUCAAAAUCGGUGAAUGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 50) |
| CS1 | 5′_2′OMe(G(ps)A(ps)C(ps))CAAUCUGACAUGCUGCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 51) |
| CD2 | 5′_2′OMe(A(ps)C(ps)A(ps))GCUGACAGGCUCGACACGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U_3′ (SEQ ID NO: 52) |
| CD2g | 5′_2′OMe(G(ps)A(ps)G(ps))AAUCAAAAUCGGUGAAUGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U 3′ (SEQ ID NO: 53) |
| CD3εg | 5′_2′OMe(A(ps)G(ps)G(ps))GCAUGUCAAUAUUACUGGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U 3′ (SEQ ID NO: 54) |
| CD5 | 5′_2′OMe(C(ps)G(ps)U(ps))UCCAACUCGAAGUGCCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps))U3′ (SEQ ID NO: 55) |
| CD5g | 5′_2′OMe(C(ps)G(ps)U(ps))uCCAACUCGAAGUGCCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 56) |

RNA; (ps) indicate phosphorothioate.
Underlined bases denote target sequence.

Step 5.

iNKT may then be transduced with a CAR targeted to, i.e., that recognizes one or more antigen or protein targets, for example with a lentivirus containing a CAR construct. Any other suitable method of transduction/transfection may be used, for example transfection using DNA-integrating viral or non-viral vectors containing transposable elements, or transient expressing of non-DNA integrating polynucleotides, such as mRNA, or insertion of CAR polynucleotide into site of nuclease activity using homologous or non-homologous recombination.

Step 6.

iNKT are then cultured to expand CAR-iNKT population. This can continue for several weeks. Regularly adding of α-GalCer loaded cells can keep the iNKT stimulated, but as with the initial stimulation, other options are available. The media typically contains high dose IL-2 (currently 200 units/ml in our protocol). IL-7, IL-15 or a combination of IL-2, IL-7 and IL-15 may also be used to expand iNKT in vitro. Analogues of these cytokines engineered to enhance potency or stability could also be used to enhance culture.

Step 7.

Optionally, cells from multiple donors may be pooled. This need not be the last step of the process, and in fact could be one of the first steps (e.g. before Step 3) if an alternative to irradiated α-GalCer pulsed negative cells is used to stimulate the iNKT cells, for example an antibody. This is because the cell population loaded with α-GalCer contains T-cells and NK cells that would become cytotoxic if samples from multiple donors were pooled.

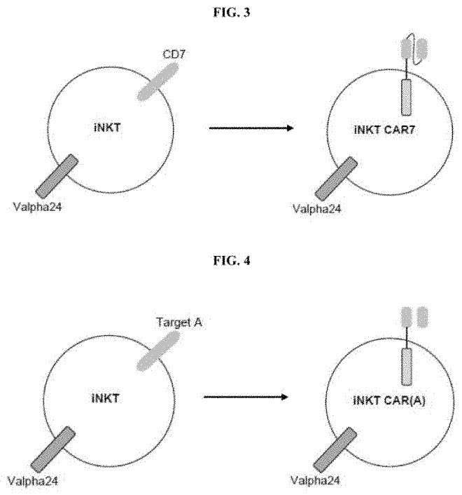

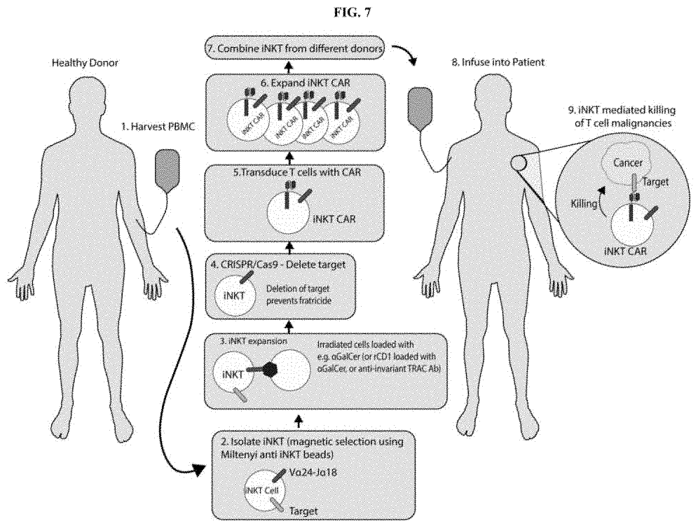

These steps are shown as flow diagrams in FIG. 1 and FIG. 7. Those of skill in the art will appreciate that some flexibility is possible in the time frames specified in FIG. 1. CAR-iNKT cells produced by these methods are shown in FIG. 3, with a CD7-targeting CAR (an iNKT-CART) and in FIG. 4, with a CAR targeting another antigen. Additional examples of iNKT-CARs are given below in Example IV.

### Example 2—Method of Making Genome-Edited Tandem CAR-iNKT Cells

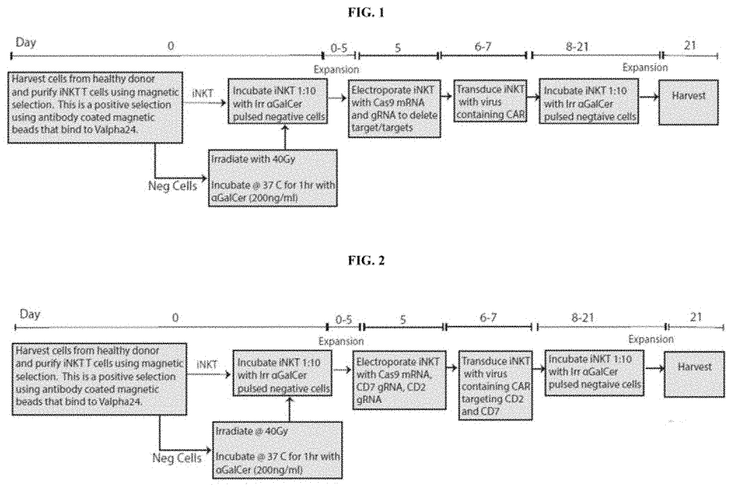

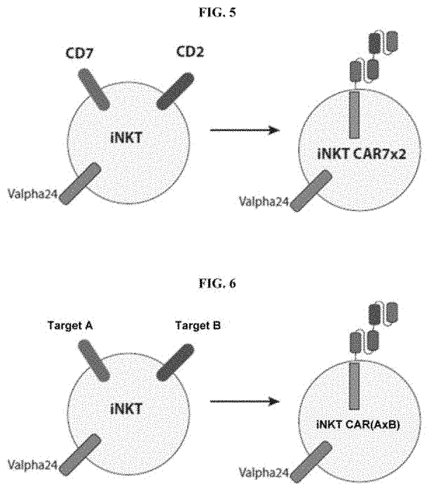

In a variation of the protocol in Example 1, a tandem CAR-iNKT recognizing two antigens can be made. In Step 4, the two antigens can be deleted from the cell surface, or suppressed as described above, but electroporation with gRNA for each of the two targets and Cas9 mRNA. In Step 5, iNKT is then transduced with a CAR that recognizes two targets. This variation is shown as a flow diagram in FIG. 2. CAR-iNKT cells produced by these methods are shown in FIG. 5, with a tandem CD2- and CD7-targeting CAR (anT-CAR7×2) and in FIG. 6, with a CAR targeting two other antigens, denoted A and B (an iNKT-CAR(A×B). Additional examples of tandem iNKT-CARs are given below in Example IV.

### Example 3—Method of Making Genome-Edited Dual CAR-iNKT Cells

In a variation of the protocol in Example 1, a dual CAR-iNKT cell targeting two antigens can be made. This variation would contain two separate CARs, each recognizing a different antigen. Additional examples of dual iNKT-CARs are given below in Example IV.

### Example 4—Genome-Edited iNKT-CAR Cells

Several types of genome-edited iNKT cells may be made using the methods above. FIG. 3 and FIG. 5 show two specific examples. Additional examples are provided herein with (deletion or suppression of the surface protein that is the antigen target of the CAR. In general, examples with deletion or suppression of antigens will have the benefit of fratricide resistance. In certain embodiments, the iNKT-CAR has deletion or suppression of the surface protein that is the antigen target of the CAR.

Additional examples of tandem and dual iNKT-CARs are provided below, with and without deletion or suppression of one or more surface proteins that is/are the antigen targets of the CARs. In general, examples with deletion or suppression of more antigens will be more likely to have the benefit of greater fratricide resistance. It should be further noted that the order in which the antigens (scFV) are oriented in the tandem CARs set forth below in Table 3 is not meant to be limiting and includes tandem iNKT-CARs in either orientation. For example, the CD2×CD3ε iNKT-tCAR is encompasses a tCAR with the orientation CD2-CD3ε or one with the orientation CD3ε-CD2.

### Example 5—Genome-Edited iNKT-Cells

TABLE 13
Examples of gene-edited iNKT cells without CARs.

| Example | Surface Protein (Antigen) Deletions |
|---|---|
| inKT-1 | CD2 |
| inKT-2 | CD3ε |
| inKT-3 | CD4 |
| inKT-4 | CD5 |
| inKT-5 | CD7 |
| inKT-6 | TRAC |
| inKT-7 | TCRβ |
| inKT-8 | CD2 and CD3ε |
| inKT-9 | CD2 and CD4 |
| inKT-10 | CD2 and CD5 |
| inKT-11 | CD2 and CD7 |
| inKT-12 | CD3ε and CD4 |
| inKT-13 | CD3ε and CD5 |
| inKT-14 | CD3ε and CD7 |
| inKT-15 | CD4 and CD5 |
| inKT-16 | CD4 and CD7 |
| inKT-17 | CD5 and CD7 |
| inKT-18 | TRAC and CD2 |
| inKT-19 | TRAC and CD3 |
| inKT-20 | TRAC and CD4 |
| inKT-21 | TRAC and CD5 |
| inKT-22 | TRAC and CD7 |
| inKT-23 | TCRβ and CD2 |
| inKT-24 | TCRβ and CD3 |
| inKT-25 | TCRβ and CD4 |
| inKT-26 | TCRβ and CD5 |
| inKT-27 | TCRβ and CD7 |
| inKT-28 | BCMA |
| inKT-29 | CS1 |
| inKT-30 | CD19 |
| inKT-31 | CD38 |
| inKT-32 | BCMA × CS1 |
| inKT-33 | BCMA × CD19 |
| inKT-34 | BCMA × CD38 |
| inKT-35 | CS1 × CD19 |
| inKT-36 | CS1 × CD38 |
| inKT-37 | CD19 × CD38 |

### Example 6—Treatment of Patient(s) with Genome-Edited iNKT Cells

Patients may be treated using cells made by the methods above, as shown in FIG. 7. For example, an expanded population of iNKT-CARs may be infused into a patient

Step 7.

As shown in Step 7 in FIG. 7, it is possible to combine iNKT from several donors prior to treatment.

Step 8.

Infuse genome-edited cells iNKT into patient.

Step 9.

iNKT target cancer cells without inducing alloreactivity. For example, iNKT-CAR7 cells would target cancer cells (and other non-cancer cells) bearing CD7 as a surface protein. iNKT-CAR7×2 cells would target cancer cells bearing CD7 and/or CD2 as a surface protein or surface proteins.

Step 10.

Optionally, co-infusion of IL-7, IL-15, IL-2, αGalCer or an analogue of any of the foregoing, alone or in combination, is expected to enhance function in vivo. The co-infusion could be slightly offset in time, as long as it would still be effective to stimulate expansion of the infused cells.

Patients treated with the iNKT-CARs disclosed herein are expected to demonstrate significantly prolonged survival, reduced tumor burden, improvement in health, and remission.

### Example 7—Biological Assays

The following assays, or variations thereon, may be used to assess efficacy of the iNKT-CARs disclosed herein.

iNKT-CAR7 for T-ALL.

Testing efficacy of iNKT-CAR7 in a xenogeneic model of T-ALL: 1×105 Click Beetle Red luciferase (CBR) labeled CCRF-CEM T-ALL (99% CD7+ by FACS) cells will be injected I.V. into NSG recipients prior to infusion of 2×106 to 1×10⁷ iNKT-CAR7 or non-targeting iNKT-CAR19 control cells i.v. on day +4. In contrast to mice receiving iNKT-CART19 or mice injected with tumor only, mice receiving iNKT-CAR7 will demonstrate significantly prolonged survival and reduced tumor burden as determined by bioluminescent imaging.

iNKT-CAR(CS1) for MM.

Testing efficacy of iNKT-CAR-CS1 in a xenogeneic model of multiple myeloma: 5×105 Click Beetle Red luciferase (CBR) labeled MM.1S (99% CS1+ by FACS) cells will be injected I.V. into NSG recipients prior to infusion of 2×106 to 1×107 iNKT-CAR-CS1 or non-targeting iNKT-CAR19 control cells i.v. on day +4, or +14 or +28. In contrast to mice receiving iNKT-CAR19 or mice injected with tumor only, mice receiving iNKT-CAR-CS1 will demonstrate significantly prolonged survival and reduced tumor burden as determined by bioluminescent imaging.

### Example 8—Off Target Analysis for gRNA Selection

Guide RNA were designed and validated for activity by Washington University Genome Engineering & iPSC. Guide RNA were designed and validated for activity by Washington University Genome Engineering & iPSC. Sequences complementary to a given gRNA may exist throughout the genome, including but not limited to the target locus. A short sequence is likelier to hybridize off-target. Similarly, some long sequences within the gRNA may have exact matches (long_0) or near matches (long_1, long_2, representing, respectively, a single or two nucleotide difference) throughout the genome. These may also hybridize off-target, in effect leading to editing of the wrong gene and diminishing editing efficiency.

Off target analysis of selected gRNA was performed for 2 exons of hCD2 (CF58 and CF59) to determine the number of sites in human genome which are an exact match or contains up to 1 or 2 mismatches, which may include the target site. The results are listed in Table 14 for Exon CF58 and Table 15 for Exon CF59.

TABLE 14
Guide RNA (gRNA) Off Target Analysis for hCD2 (Exon CF58)

| Name | gRNA | long_0 | long_1 | long_2 | short_0 | SNP |
|---|---|---|---|---|---|---|
| CF58.CD2.g1 | CAAAGAGATTACGAATGCCTNGG | 1 | 1 | 1 | 3 | NA |
| | (SEQ ID NO: 57) | | | | | |
| CF58.CD2.g23 | CAAGGCATTCGTAATCTCTTNGG | 1 | 1 | 1 | 5 | NA |
| | (SEQ ID NO: 58) | | | | | |
| CF58.CD2.g18 | CTTGTAGATATCCTGATCATNGG | 1 | 1 | 1 | 13 | NA |
| | (SEQ ID NO: 59) | | | | | |
| CF58.CD2.g8 | CTTGGGTCAGGACATCAACTNGG | 1 | 1 | 1 | 14 | NA |
| | (SEQ ID NO: 60) | | | | | |
| CF58.CD2.g14 | CGATGATCAGGATATCTACANGG | 1 | 1 | 1 | 17 | NA |
| | (SEQ ID NO: 61) | | | | | |
| CF58.CD2.g2 | TTACGAATGCCTTGGAAACCNGG | 1 | 1 | 1 | 27 | NA |
| | (SEQ ID NO: 62) | | | | | |
| CF58.CD2.g3 | TACGAATGCCTTGGAAACCTNGG | 1 | 1 | 1 | 34 | NA |
| | (SEQ ID NO: 63) | | | | | |
| CF58.CD2.g4 | ACGAATGCCTTGGAAACCTGNGG | 1 | 1 | 1 | 40 | NA |
| | (SEQ ID NO: 64) | | | | | |
| CF58.CD2.g10 | TGATATTGACGATATAAAATNGG | 1 | 1 | 2 | 3 | NA |
| | (SEQ ID NO: 65) | | | | | |
| CF58.CD2.g9 | ATGATATTGACGATATAAAANGG | 1 | 1 | 2 | 4 | NA |
| | (SEQ ID NO: 66) | | | | | |
| CF58.CD2.g13 | GCATCTGAAGACCGATGATCNGG | 1 | 1 | 2 | 4 | NA |
| | (SEQ ID NO: 67) | | | | | |
| CF58.CD2.g7 | AACCTGGGGTGCCTTGGGTCNGG | 1 | 1 | 2 | 22 | NA |
| | (SEQ ID NO: 68) | | | | | |
| CF58.CD2.g6 | TTGGAAACCTGGGGTGCCTTNGG | 1 | 1 | 2 | 33 | NA |
| | (SEQ ID NO: 69) | | | | | |
| CF58.CD2.g15 | GTATCAATATATGATACAAANGG | 1 | 1 | 2 | 35 | NA |
| | (SEQ ID NO: 70) | | | | | |
| CF58.CD2.g22 | CAAGGCACCCCAGGTTTCCANGG | 1 | 1 | 2 | 45 | NA |
| | (SEQ ID NO: 71) | | | | | |
| CF58.CD2.g5 | CTTGGAAACCTGGGGTGCCTNGG | 1 | 1 | 2 | 62 | NA |
| | (SEQ ID NO: 72) | | | | | |
| CF58.CD2.g19 | TCATCACTCATTTGAAAACTNGG | 1 | 1 | 3 | 56 | NA |
| | (SEQ ID NO: 73) | | | | | |
| CF58.CD2.g20 | CAAGTTGATGTCCTGACCCANGG | 1 | 1 | 4 | 27 | NA |
| | (SEQ ID NO: 74) | | | | | |
| CF58.CD2.g21 | GTCCTGACCCAAGGCACCCCNGG | 1 | 1 | 4 | 33 | NA |
| | (SEQ ID NO: 75) | | | | | |
| CF58.CD2.g17 | ATATTTGATTTGAAGATTCANGG | 1 | 1 | 6 | 35 | NA |
| | (SEQ ID NO: 76) | | | | | |
| CF58.CD2.g16 | TACAAAAGGAAAAAATGTGTNGG | 1 | 1 | 7 | 64 | NA |
| | (SEQ ID NO: 77) | | | | | |
| CF58.CD2.g12 | ACATATAAGCTATTTAAAAANGG | 1 | 1 | 8 | 58 | NA |
| | (SEQ ID NO: 78) | | | | | |
| CF58.CD2.g11 | AAAAGAGAAAGAGACTTTCANGG | 1 | 1 | 15 | 42 | NA |
| | (SEQ ID NO: 79) | | | | | |

TABLE 15
Guide RNA (gRNA) Off Target Analysis for hCD2 (CF59)

| Name | gRNA | long_0 | long_1 | long_2 | short_0 | SNP |
|---|---|---|---|---|---|---|
| CF59.CD2.g20 | CTTGATACAGGTTTAATTCGNGG | 1 | 1 | 1 | 2 | NA |
| | (SEQ ID NO: 80) | | | | | |
| CF59.CD2.g13 | ACAGCTGACAGGCTCGACACNGG | 1 | 1 | 1 | 4 | NA |
| | (SEQ ID NO: 81) | | | | | |
| CF59.CD2.g17 | GATGTTTCCCATCTTGATACNGG | 1 | 1 | 1 | 8 | NA |
| | (SEQ ID NO: 82) | | | | | |
| CF59.CD2.g12 | GTCGAGCCTGTCAGCTGTCCNGG | 1 | 1 | 1 | 24 | NA |
| | (SEQ ID NO: 83) | | | | | |
| CF59.CD2.g10 | CAAAATTCAAGTGCACAGCANGG | 1 | 1 | 1 | 33 | NA |
| | (SEQ ID NO: 84) | | | | | |
| CF59.CD2.g16 | GAATTTTGCACTCAGGCTGGNGG | 1 | 1 | 1 | 245 | NA |
| | (SEQ ID NO: 85) | | | | | |
| CF59.CD2.g4 | GAATTAAACCTGTATCAAGANGG | 1 | 1 | 2 | 7 | NA |
| | (SEQ ID NO: 86) | | | | | |
| CF59.CD2.g5 | AATTAAACCTGTATCAAGATNGG | 1 | 1 | 2 | 7 | NA |
| | (SEQ ID NO: 87) | | | | | |
| CF59.CD2.g21 | AGTTCCATTCATTACCTCACNGG | 1 | 1 | 2 | 14 | NA |
| | (SEQ ID NO: 88) | | | | | |
| CF59.CD2.g8 | AGAGGGTCATCACACACAAGNGG | 1 | 1 | 2 | 20 | NA |
| | (SEQ ID NO: 89) | | | | | |
| CF59.CD2.g25 | ATACAAGTCCAGGAGATCTTNGG | 1 | 1 | 2 | 21 | NA |
| | (SEQ ID NO: 90) | | | | | |
| CF59.CD2.g19 | TCTTGATACAGGTTTAATTCNGG | 1 | 1 | 2 | 25 | NA |
| | (SEQ ID NO: 91) | | | | | |
| CF59.CD2.g3 | CTGACCTGTGAGGTAATGAANGG | 1 | 1 | 2 | 29 | NA |
| | (SEQ ID NO: 92) | | | | | |
| CF59.CD2.g7 | ACATCTAAAACTTTCTCAGANGG | 1 | 1 | 2 | 41 | NA |
| | (SEQ ID NO: 93) | | | | | |
| CF59.CD2.g9 | GCAAAATTCAAGTGCACAGCNGG | 1 | 1 | 2 | 46 | NA |
| | (SEQ ID NO: 94) | | | | | |
| CF59.CD2.g24 | GGTTGTGTTGATACAAGTCCNGG | 1 | 1 | 3 | 8 | NA |
| | (SEQ ID NO: 95) | | | | | |
| CF59.CD2.g18 | ATCTTGATACAGGTTTAATTNGG | 1 | 1 | 3 | 24 | NA |
| | (SEQ ID NO: 96) | | | | | |
| CF59.CD2.g23 | ATTCATTACCTCACAGGTCANGG | 1 | 1 | 3 | 35 | NA |
| | (SEQ ID NO: 97) | | | | | |
| CF59.CD2.g6 | AACATCTAAAACTTTCTCAGNGG | 1 | 1 | 3 | 43 | NA |
| | (SEQ ID NO: 98) | | | | | |
| CF59.CD2.g11 | AGCAGGGAACAAAGTCAGCANGG | 1 | 1 | 3 | 45 | NA |
| | (SEQ ID NO: 99) | | | | | |
| CF59.CD2.g2 | CAACACAACCCTGACCTGTGNGG | 1 | 1 | 3 | 47 | NA |
| | (SEQ ID NO: 100) | | | | | |
| CF59.CD2.g15 | CTTGAATTTTGCACTCAGGCNGG | 1 | 1 | 4 | 21 | NA |
| | (SEQ ID NO: 101) | | | | | |
| CF59.CD2.g22 | CATTCATTACCTCACAGGTCNGG | 1 | 1 | 10 | 29 | NA |
| | (SEQ ID NO: 102) | | | | | |
| CF59.CD2.g14 | TGCACTTGAATTTTGCACTCNGG | 1 | 2 | 3 | 26 | NA |
| | (SEQ ID NO: 103) | | | | | |
| CF59.CD2.g1 | TCTCAAAACCAAAGATCTCCNGG | 1 | 2 | 5 | 19 | NA |
| | (SEQ ID NO: 104) | | | | | |

The gRNA sequences in Table 14 and Table 15 were normalized (% Normalization to NHEJ) for gRNA activity via next generation sequencing (NGS). GFP was used as a control. Following sequencing analysis, the following gRNAs were recommended based on off-target profile: CF58.CD2.g1 (41.2%), CF58.CD2.g23 (13.2%), CF59.CD2.g20 (26.6%), CF59.CD2.g13 (66.2%), CF59.CD2.g17 (17.5%). Guide RNA (gRNA) with normalized NHEJ frequencies equal to or greater than 15% are good candidates for cell line and animal model creation projects.

Off target analysis of selected gRNA was performed for hCD3E to determine the number of sites in human genome which are an exact match or contains up to 1 or 2 mismatches, which may include the target site. The results are listed in Table 16 for hCD3E.

TABLE 16
Guide RNA (gRNA) Off Target Analysis for hCD3E

| Name | gRNA | long_0 | long_1 | long_2 | long_3 | short_0 | SNP |
|---|---|---|---|---|---|---|---|
| MS1044.CD3E.sp2 | TTGACATGCCCTCAGTATCCNGG | 1 | 1 | 1 | 21 | 73 | NA |
| | (SEQ ID NO: 105) | | | | | | |
| MS1044.CD3E.sp17 | CTGGATTACCTCTTGCCCTCNGG | 1 | 1 | 1 | 24 | 114 | NA |
| | (SEQ ID NO: 106) | | | | | | |
| MS1044.CD3E.sp28 | GAGATGGAGACTTTATATGCNGG | 1 | 1 | 1 | 30 | 44 | NA |
| | (SEQ ID NO: 107) | | | | | | |
| MS1044.CD3E.sp29 | AGATGGAGACTTTATATGCTNGG | 1 | 1 | 1 | 33 | 55 | NA |
| | (SEQ ID NO: 108) | | | | | | |
| MS1044.CD3E.sp26 | AGGGCATGTCAATATTACTGNGG | 1 | 1 | 1 | 23 | 60 | NA |
| | (SEQ ID NO: 109) | | | | | | |
| MS1044.CD3E.sp30 | GATGGAGACTTTATATGCTGNGG | 1 | 1 | 2 | 26 | 64 | NA |
| | (SEQ ID NO: 110) | | | | | | |
| MS1044.CD3E.sp12 | TATTATGTCTGCTACCCCAGNGG | 1 | 1 | 2 | 20 | 61 | NA |
| | (SEQ ID NO: 111) | | | | | | |
| MS1044.CD3E.sp23 | TGCCATAGTATTTCAGATCCNGG | 1 | 1 | 2 | 21 | 55 | NA |
| | (SEQ ID NO: 112) | | | | | | |
| MS1044.CD3E.sp18 | AGATAAAAGTTCGCATCTTCNGG | 1 | 1 | 2 | 33 | 6 | NA |
| | (SEQ ID NO: 113) | | | | | | |
| MS1044.CD3E.sp22 | CTGAAAATTCCTTCAGTGACNGG | 1 | 1 | 2 | 44 | 60 | NA |
| | (SEQ ID NO: 114) | | | | | | |
| MS1044.CD3E.sp16 | CTGAGGGCAAGAGGTAATCCNGG | 1 | 1 | 3 | 30 | 41 | NA |
| | (SEQ ID NO: 115) | | | | | | |
| MS1044.CD3E.sp25 | TTTCAGATCCAGGATACTGANGG | 1 | 1 | 3 | 38 | 63 | NA |
| | (SEQ ID NO: 116) | | | | | | |
| MS1044.CD3E.sp15 | TATCTCTACCTGAGGGCAAGNGG | 1 | 1 | 3 | 22 | 134 | NA |
| | (SEQ ID NO: 117) | | | | | | |
| MS1044.CD3E.sp9 | TGAGGATCACCTGTCACTGANGG | 1 | 1 | 3 | 44 | 54 | NA |
| | (SEQ ID NO: 118) | | | | | | |

The gRNA sequences in Table 16 were normalized (% Normalization to NHEJ) for gRNA activity via next generation sequencing (NGS). GFP was used as a control. Following sequencing analysis, the following gRNAs were recommended based on off-target profile: MS1044.CD3E.sp28 (>15%) and MS1044.CD3E.sp12 (>15%). Guide RNA (gRNA) with normalized NHEJ frequencies equal to or greater than 15% are good candidates for cell line and animal model creation projects.

Off target analysis of selected gRNA was performed for 3 exons of hCD5 (Exon 3, Exon 4, and Exon 5) to determine the number of sites in human genome which are an exact match or contains up to 1 or 2 mismatches, which may include the target site. The results are listed in Table 17 for Exon 3, Table 18 for Exon 4, and Table 19 for Exon 5.

TABLE 17
Guide RNA (gRNA) Off Target Analysis for hCD5 (Exon 3)

| Name | gRNA | long_0 | long_1 | long_2 | short_0 | SNP |
|---|---|---|---|---|---|---|
| SP597.CD5.g22 | AATCATCTGCTACGGACAACNGG | 1 | 1 | 1 | 1 | NA |
| | (SEQ ID NO: 119) | | | | | |
| SP597.CD5.g39 | GCAGACTTTTGACGCTTGACNGG | 1 | 1 | 1 | 1 | NA |
| | (SEQ ID NO: 120) | | | | | |
| SP597.CD5.g1 | CCGTTCCAACTCGAAGTGCCNGG | 1 | 1 | 1 | 2 | NA |
| | (SEQ ID NO: 121) | | | | | |
| SP597.CD5.g2 | CGTTCCAACTCGAAGTGCCANGG | 1 | 1 | 1 | 2 | NA |
| | (SEQ ID NO: 122) | | | | | |
| SP597.CD5.g50 | CTGGCACTTCGAGTTGGAACNGG | 1 | 1 | 1 | 2 | NA |
| | (SEQ ID NO: 123) | | | | | |
| SP597.CD5.g17 | GTCTGCCAGCGGCTGAACTGNGG | 1 | 1 | 1 | 3 | NA |
| | (SEQ ID NO: 124) | | | | | |
| SP597.CD5.g23 | ATCATCTGCTACGGACAACTNGG | 1 | 1 | 1 | 3 | NA |
| | (SEQ ID NO: 125) | | | | | |
| SP597.CD5.g41 | AGACTTTTGACGCTTGACTGNGG | 1 | 1 | 1 | 3 | NA |
| | (SEQ ID NO: 126) | | | | | |
| SP597.CD5.g40 | CAGACTTTTGACGCTTGACTNGG | 1 | 1 | 1 | 5 | NA |
| | (SEQ ID NO: 127) | | | | | |
| SP597.CD5.g49 | CCTGGCACTTCGAGTTGGAANGG | 1 | 1 | 1 | 5 | NA |
| | (SEQ ID NO: 128) | | | | | |
| SP597.CD5.g38 | GCACCCCACAGTTCAGCCGCNGG | 1 | 1 | 1 | 8 | NA |
| | (SEQ ID NO: 129) | | | | | |
| SP597.CD5.g46 | CCTTGAGGTAGACCTCCAGCNGG | 1 | 1 | 1 | 9 | NA |
| | (SEQ ID NO: 130) | | | | | |
| SP597.CD5.g7 | AGGTCTACCTCAAGGACGGANGG | 1 | 1 | 1 | 11 | NA |
| | (SEQ ID NO: 131) | | | | | |
| SP597.CD5.g51 | TGGAACGGGTGAGCCTTGCCNGG | 1 | 1 | 1 | 13 | NA |
| | (SEQ ID NO: 132) | | | | | |
| SP597.CD5.g20 | TGTGGGGTGCCCTTAAGCCTNGG | 1 | 1 | 1 | 19 | NA |
| | (SEQ ID NO: 133) | | | | | |
| SP597.CD5.g16 | AAGCGTCAAAAGTCTGCCAGNGG | 1 | 1 | 1 | 20 | NA |
| | (SEQ ID NO: 134) | | | | | |
| SP597.CD5.g29 | TAGCAGATGATTGAGCTCTGNGG | 1 | 1 | 1 | 25 | NA |
| | (SEQ ID NO: 135) | | | | | |
| SP597.CD5.g30 | GATTGAGCTCTGAGGTGTGTNGG | 1 | 1 | 1 | 33 | NA |
| | (SEQ ID NO: 136) | | | | | |
| SP597.CD5.g13 | GGGGCCGGAGCTCCAAGCAGNGG | 1 | 1 | 1 | 42 | NA |
| | (SEQ ID NO: 137) | | | | | |
| SP597.CD5.g33 | GGTGTGTAGGTGACAAGGAANGG | 1 | 1 | 1 | 48 | NA |
| | (SEQ ID NO: 138) | | | | | |
| SP597.CD5.g15 | CCGGAGCTCCAAGCAGTGGGNGG | 1 | 1 | 1 | 58 | NA |
| | (SEQ ID NO: 139) | | | | | |
| SP597.CD5.g47 | GGTAGACCTCCAGCTGGCCCNGG | 1 | 1 | 1 | 78 | NA |
| | (SEQ ID NO: 140) | | | | | |
| SP597.CD5.g3 | CTCGAAGTGCCAGGGCCAGCNGG | 1 | 1 | 1 | 121 | NA |
| | (SEQ ID NO: 141) | | | | | |
| SP597.CD5.g48 | CTGGCCCTGGCACTTCGAGTNGG | 1 | 1 | 2 | 1 | NA |
| | (SEQ ID NO: 142) | | | | | |
| SP597.CD5.g18 | TCTGCCAGCGGCTGAACTGTNGG | 1 | 1 | 2 | 5 | NA |
| | (SEQ ID NO: 143) | | | | | |
| SP597.CD5.g45 | CCATGTGCCATCCGTCCTTGNGG | 1 | 1 | 2 | 5 | NA |
| | (SEQ ID NO: 144) | | | | | |
| SP597.CD5.g5 | CCAGCTGGAGGTCTACCTCANGG | 1 | 1 | 2 | 14 | NA |
| | (SEQ ID NO: 145) | | | | | |
| SP597.CD5.g31 | TCTGAGGTGTGTAGGTGACANGG | 1 | 1 | 2 | 18 | NA |
| | (SEQ ID NO: 146) | | | | | |
| SP597.CD5.g37 | AGGAAGGGGCCAAGGCTTAANGG | 1 | 1 | 2 | 18 | NA |
| | (SEQ ID NO: 147) | | | | | |
| SP597.CD5.g21 | CAGAGCTCAATCATCTGCTANGG | 1 | 1 | 2 | 19 | NA |
| | (SEQ ID NO: 148) | | | | | |
| SP597.CD5.g14 | GGGCCGGAGCTCCAAGCAGTNGG | 1 | 1 | 2 | 23 | NA |
| | (SEQ ID NO: 149) | | | | | |
| SP597.CD5.g43 | CCTCCCACTGCTTGGAGCTCNGG | 1 | 1 | 2 | 30 | NA |
| | (SEQ ID NO: 150) | | | | | |
| SP597.CD5.g44 | TGGAGCTCCGGCCCCAGCTCNGG | 1 | 1 | 2 | 38 | NA |
| | (SEQ ID NO: 151) | | | | | |
| SP597.CD5.g34 | GTGTGTAGGTGACAAGGAAGNGG | 1 | 1 | 2 | 48 | NA |
| | (SEQ ID NO: 152) | | | | | |
| SP597.CD5.g11 | ATGGTTTGCAGCCAGAGCTGNGG | 1 | 1 | 2 | 108 | NA |
| | (SEQ ID NO: 153) | | | | | |
| SP597.CD5.g6 | CTGGAGGTCTACCTCAAGGANGG | 1 | 1 | 3 | 16 | NA |
| | (SEQ ID NO: 154) | | | | | |
| SP597.CD5.g19 | CTGCCAGCGGCTGAACTGTGNGG | 1 | 1 | 3 | 25 | NA |
| | (SEQ ID NO: 155) | | | | | |
| SP597.CD5.g25 | AATGACATGTGTCACTCTCTNGG | 1 | 1 | 3 | 25 | NA |
| | (SEQ ID NO: 156) | | | | | |
| SP597.CD5.g9 | ACATGGTTTGCAGCCAGAGCNGG | 1 | 1 | 3 | 30 | NA |
| | (SEQ ID NO: 157) | | | | | |
| SP597.CD5.g10 | CATGGTTTGCAGCCAGAGCTNGG | 1 | 1 | 3 | 52 | NA |
| | (SEQ ID NO: 158) | | | | | |
| SP597.CD5.g26 | GACACATGTCATTTCTGCTGNGG | 1 | 1 | 3 | 53 | NA |
| | (SEQ ID NO: 159) | | | | | |
| SP597.CD5.g42 | ACTGGGGTCCTCCCACTGCTNGG | 1 | 1 | 3 | 91 | NA |
| | (SEQ ID NO: 160) | | | | | |
| SP597.CD5.g8 | CCTCAAGGACGGATGGCACANGG | 1 | 1 | 4 | 5 | NA |
| | (SEQ ID NO: 161) | | | | | |
| SP597.CD5.g32 | AGGTGTGTAGGTGACAAGGANGG | 1 | 1 | 4 | 49 | NA |
| | (SEQ ID NO: 162) | | | | | |
| SP597.CD5.g36 | AAGGAAGGGGCCAAGGCTTANGG | 1 | 1 | 5 | 16 | NA |
| | (SEQ ID NO: 163) | | | | | |
| SP597.CD5.g4 | GAAGTGCCAGGGCCAGCTGGNGG | 1 | 1 | 5 | 93 | NA |
| | (SEQ ID NO: 164) | | | | | |
| SP597.CD5.g12 | TTTGCAGCCAGAGCTGGGGCNGG | 1 | 1 | 8 | 257 | NA |
| | (SEQ ID NO: 165) | | | | | |
| SP597.CD5.g24 | AAATGACATGTGTCACTCTCNGG | 1 | 1 | 10 | 33 | NA |
| | (SEQ ID NO: 166) | | | | | |
| SP597.CD5.g35 | AGGTGACAAGGAAGGGGCCANGG | 1 | 1 | 10 | 202 | NA |
| | (SEQ ID NO: 167) | | | | | |
| SP597.CD5.g27 | ATTTCTGCTGTGGCTGCAGTNGG | 1 | 2 | 4 | 70 | NA |
| | (SEQ ID NO: 168) | | | | | |
| SP597.CD5.g28 | GCTGTGGCTGCAGTTGGAGANGG | 1 | 2 | 19 | 49 | NA |
| | (SEQ ID NO: 169) | | | | | |

TABLE 18
Guide RNA (gRNA) Off Target Analysis for hCD5 (Exon 4)

| Name | gRNA | long_0 | long_1 | long_2 | short_0 | SNP |
|---|---|---|---|---|---|---|
| SP598.CD5.g10 | GGCGGGGGCCTTGTCGTTGGNGG | 1 | 1 | 1 | 1 | NA |
| | (SEQ ID NO: 170) | | | | | |
| SP598.CD5.g7 | CTCTGGAGTTGTGGTGGGCGNGG | 1 | 1 | 1 | 16 | NA |
| | (SEQ ID NO: 171) | | | | | |
| SP598.CD5.g8 | TCTGGAGTTGTGGTGGGCGGNGG | 1 | 1 | 1 | 40 | NA |
| | (SEQ ID NO: 172) | | | | | |
| SP598.CD5.g12 | CGTTGGAGGTGTTGTCTTCTNGG | 1 | 1 | 1 | 46 | NA |
| | (SEQ ID NO: 173) | | | | | |
| SP598.CD5.g1 | AGACAACACCTCCAACGACANGG | 1 | 1 | 2 | 2 | NA |
| | (SEQ ID NO: 174) | | | | | |
| SP598.CD5.g9 | GTGGGCGGGGGCCTTGTCGTNGG | 1 | 1 | 2 | 5 | NA |
| | (SEQ ID NO: 175) | | | | | |
| SP598.CD5.g11 | TCGTTGGAGGTGTTGTCTTCNGG | 1 | 1 | 2 | 13 | NA |
| | (SEQ ID NO: 176) | | | | | |
| SP598.CD5.g2 | ACCACAACTCCAGAGCCCACNGG | 1 | 1 | 2 | 60 | NA |
| | (SEQ ID NO: 177) | | | | | |
| SP598.CD5.g6 | GCTCTGGAGTTGTGGTGGGCNGG | 1 | 1 | 4 | 74 | NA |
| | (SEQ ID NO: 178) | | | | | |
| SP598.CD5.g4 | GTGGGCTCTGGAGTTGTGGTNGG | 1 | 1 | 6 | 35 | NA |
| | (SEQ ID NO: 179) | | | | | |
| SP598.CD5.g3 | TGTGGGCTCTGGAGTTGTGGNGG | 1 | 1 | 8 | 54 | NA |
| | (SEQ ID NO: 180) | | | | | |
| SP598.CD5.g13 | GTTGGAGGTGTTGTCTTCTGNGG | 1 | 2 | 2 | 48 | NA |
| | (SEQ ID NO: 181) | | | | | |
| SP598.CD5.g5 | GGCTCTGGAGTTGTGGTGGGNGG | 1 | 3 | 9 | 51 | NA |
| | (SEQ ID NO: 182) | | | | | |

TABLE 19
Guide RNA (gRNA) Off Target Analysis for hCD5 (Exon 5)

| Name | gRNA | long_0 | long_1 | long_2 | short_0 | SNP |
|---|---|---|---|---|---|---|
| SP599.CD5.g58 | CATAGCTGATGGTACCCCCCNGG | 1 | 1 | 1 | 1 | NA |
| | (SEQ ID NO: 183) | | | | | |
| SP599.CD5.g5 | CGGCCAGCACTGTGCCGGCGNGG | 1 | 1 | 1 | 2 | NA |
| | (SEQ ID NO: 184) | | | | | |
| SP599.CD5.g30 | CAAGAACTCGGCCACTTTTCNGG | 1 | 1 | 1 | 6 | NA |
| | (SEQ ID NO: 185) | | | | | |
| SP599.CD5.g44 | GGTGTTCCCGTGGCTCCCCTNGG | 1 | 1 | 1 | 11 | rs2241002: 0.158 |
| | (SEQ ID NO: 186) | | | | | |
| SP599.CD5.g6 | CCAGCACTGTGCCGGCGTGGNGG | 1 | 1 | 1 | 13 | NA |
| | (SEQ ID NO: 187) | | | | | |
| SP599.CD5.g42 | GGCAAGGGCTGGTGTTCCCGNGG | 1 | 1 | 1 | 13 | NA |
| | (SEQ ID NO: 188) | | | | | |
| SP599.CD5.g7 | GGCGTGGTGGAGTTCTACAGNGG | 1 | 1 | 1 | 14 | NA |
| | (SEQ ID NO: 189) | | | | | |
| SP599.CD5.g60 | CCACCACGCCGGCACAGTGCNGG | 1 | 1 | 1 | 15 | NA |
| | (SEQ ID NO: 190) | | | | | |
| SP599.CD5.g8 | GGAGTTCTACAGCGGCAGCCNGG | 1 | 1 | 1 | 17 | NA |
| | (SEQ ID NO: 191) | | | | | |
| SP599.CD5.g11 | GTTCTACAGCGGCAGCCTGGNGG | 1 | 1 | 1 | 18 | NA |
| | (SEQ ID NO: 192) | | | | | |
| SP599.CD5.g25 | ACCAGCCCTTGCCAATCCAANGG | 1 | 1 | 1 | 20 | NA |
| | (SEQ ID NO: 193) | | | | | |
| SP599.CD5.g10 | AGTTCTACAGCGGCAGCCTGNGG | 1 | 1 | 1 | 24 | NA |
| | (SEQ ID NO: 194) | | | | | |
| SP599.CD5.g55 | CCAGGTCCTGGGTCTTGTCCNGG | 1 | 1 | 1 | 25 | NA |
| | (SEQ ID NO: 195) | | | | | |
| SP599.CD5.g43 | TGGTGTTCCCGTGGCTCCCCNGG | 1 | 1 | 1 | 25 | rs2241002: 0.158 |
| | (SEQ ID NO: 196) | | | | | |
| SP599.CD5.g9 | GAGTTCTACAGCGGCAGCCTNGG | 1 | 1 | 1 | 26 | NA |
| | (SEQ ID NO: 197) | | | | | |
| SP599.CD5.g26 | GAACTCAAGCTGTACCTCCCNGG | 1 | 1 | 1 | 29 | NA |
| | (SEQ ID NO: 198) | | | | | |
| SP599.CD5.g31 | AAGAACTCGGCCACTTTTCTNGG | 1 | 1 | 1 | 29 | NA |
| | (SEQ ID NO: 199) | | | | | |
| SP599.CD5.g41 | TCCATTGGATTGGCAAGGGCNGG | 1 | 1 | 1 | 32 | NA |
| | (SEQ ID NO: 200) | | | | | |
| SP599.CD5.g12 | TTCTACAGCGGCAGCCTGGGNGG | 1 | 1 | 1 | 33 | NA |
| | (SEQ ID NO: 201) | | | | | |
| SP599.CD5.g32 | AGAACTCGGCCACTTTTCTGNGG | 1 | 1 | 1 | 37 | NA |
| | (SEQ ID NO: 202) | | | | | |
| SP599.CD5.g49 | GCTTCAAGAAGGAGCCACACNGG | 1 | 1 | 1 | 48 | NA |
| | (SEQ ID NO: 203) | | | | | |
| SP599.CD5.g39 | GATCTTCCATTGGATTGGCANGG | 1 | 1 | 2 | 7 | NA |
| | (SEQ ID NO: 204) | | | | | |
| SP599.CD5.g59 | GCTGTAGAACTCCACCACGCNGG | 1 | 1 | 2 | 11 | NA |
| | (SEQ ID NO: 205) | | | | | |
| SP599.CD5.g57 | GTCCTGGGCCTCATAGCTGANGG | 1 | 1 | 2 | 13 | NA |
| | (SEQ ID NO: 206) | | | | | |
| SP599.CD5.g14 | TACCATCAGCTATGAGGCCCNGG | 1 | 1 | 2 | 14 | NA |
| | (SEQ ID NO: 207) | | | | | |
| SP599.CD5.g13 | GGGGGGTACCATCAGCTATGNGG | 1 | 1 | 2 | 16 | NA |
| | (SEQ ID NO: 208) | | | | | |
| SP599.CD5.g35 | CCTGAAGCAATGCTCCAGGGNGG | 1 | 1 | 2 | 18 | NA |
| | (SEQ ID NO: 209) | | | | | |
| SP599.CD5.g33 | TTTTCCTGAAGCAATGCTCCNGG | 1 | 1 | 2 | 24 | NA |
| | (SEQ ID NO: 210) | | | | | |
| SP599.CD5.g48 | CTCTGGCAGATGCTTCAAGANGG | 1 | 1 | 2 | 25 | NA |
| | (SEQ ID NO: 211) | | | | | |
| SP599.CD5.g53 | AGAGGAAGTTCTCCAGGTCCNGG | 1 | 1 | 2 | 53 | NA |
| | (SEQ ID NO: 212) | | | | | |
| SP599.CD5.g4 | TCTGGCGGCCAGCACTGTGCNGG | 1 | 1 | 2 | 166 | NA |
| | (SEQ ID NO: 213) | | | | | |
| SP599.CD5.g37 | TTGAGTTCTGGATCTTCCATNGG | 1 | 1 | 3 | 9 | NA |
| | (SEQ ID NO: 214) | | | | | |
| SP599.CD5.g38 | TTCTGGATCTTCCATTGGATNGG | 1 | 1 | 3 | 13 | NA |
| | (SEQ ID NO: 215) | | | | | |
| SP599.CD5.g40 | ATCTTCCATTGGATTGGCAANGG | 1 | 1 | 3 | 18 | NA |
| | (SEQ ID NO: 216) | | | | | |
| SP599.CD5.g50 | TCAAGAAGGAGCCACACTGGNGG | 1 | 1 | 3 | 31 | NA |
| | (SEQ ID NO: 217) | | | | | |
| SP599.CD5.g36 | GGGAGGTACAGCTTGAGTTCNGG | 1 | 1 | 3 | 37 | NA |
| | (SEQ ID NO: 218) | | | | | |
| SP599.CD5.g45 | CCCGTGGCTCCCCTGGGTCTNGG | 1 | 1 | 3 | 43 | rs2241002: 0.158 |
| | (SEQ ID NO: 219) | | | | | |
| SP599.CD5.g16 | CCAGGACAAGACCCAGGACCNGG | 1 | 1 | 3 | 57 | NA |
| | (SEQ ID NO: 220) | | | | | |
| SP599.CD5.g17 | CTCTGCAACAACCTCCAGTGNGG | 1 | 1 | 3 | 67 | NA |
| | (SEQ ID NO: 221) | | | | | |
| SP599.CD5.g52 | TGTTGCAGAGGAAGTTCTCCNGG | 1 | 1 | 3 | 236 | NA |
| | (SEQ ID NO: 222) | | | | | |
| SP599.CD5.g56 | CAGGTCCTGGGTCTTGTCCTNGG | 1 | 1 | 4 | 24 | NA |
| | (SEQ ID NO: 223) | | | | | |
| SP599.CD5.g15 | TGAGGCCCAGGACAAGACCCNGG | 1 | 1 | 4 | 30 | NA |
| | (SEQ ID NO: 224) | | | | | |
| SP599.CD5.g61 | CTGTGCCACCAGCTGCAGCCNGG | 1 | 1 | 4 | 133 | NA |
| | (SEQ ID NO: 225) | | | | | |
| SP599.CD5.g62 | TGTGCCACCAGCTGCAGCCTNGG | 1 | 1 | 4 | 139 | NA |
| | (SEQ ID NO: 226) | | | | | |
| SP599.CD5.g19 | CATCTGCCAGAGACTGAGGCNGG | 1 | 1 | 4 | 1253 | NA |
| | (SEQ ID NO: 227) | | | | | |
| SP599.CD5.g2 | CTGCAGCTGGTGGCACAGTCNGG | 1 | 1 | 5 | 17 | NA |
| | (SEQ ID NO: 228) | | | | | |
| SP599.CD5.g51 | CACACTGGAGGTTGTTGCAGNGG | 1 | 1 | 5 | 28 | NA |
| | (SEQ ID NO: 229) | | | | | |
| SP599.CD5.g3 | CAGCTGGTGGCACAGTCTGGNGG | 1 | 1 | 5 | 31 | NA |
| | (SEQ ID NO: 230) | | | | | |
| SP599.CD5.g29 | AGCAAAGGAGGGCAAGAACTNGG | 1 | 1 | 6 | 53 | NA |
| | (SEQ ID NO: 231) | | | | | |
| SP599.CD5.g54 | GAGGAAGTTCTCCAGGTCCTNGG | 1 | 1 | 6 | 53 | NA |
| | (SEQ ID NO: 232) | | | | | |
| SP599.CD5.g63 | GCCACCAGCTGCAGCCTGGGNGG | 1 | 1 | 6 | 287 | NA |
| | (SEQ ID NO: 233) | | | | | |
| SP599.CD5.g20 | GCAGGCAGAGCCCAAGACCCNGG | 1 | 1 | 7 | 40 | rs2241002: 0.158 |
| | (SEQ ID NO: 234) | | | | | |
| SP599.CD5.g21 | CAGGCAGAGCCCAAGACCCANGG | 1 | 1 | 8 | 45 | rs2241002: 0.158 |
| | (SEQ ID NO: 235) | | | | | |
| SP599.CD5.g1 | TCCTCCCAGGCTGCAGCTGGNGG | 1 | 1 | 8 | 140 | NA |
| | (SEQ ID NO: 236) | | | | | |
| SP599.CD5.g47 | GCTCTGCCTGCCTCAGTCTCNGG | 1 | 1 | 26 | 412 | NA |
| | (SEQ ID NO: 237) | | | | | |
| SP599.CD5.g27 | CCTCCCTGGAGCATTGCTTCNGG | 1 | 2 | 3 | 22 | NA |
| | (SEQ ID NO: 238) | | | | | |
| SP599.CD5.g34 | TTTCCTGAAGCAATGCTCCANGG | 1 | 2 | 4 | 32 | NA |
| | (SEQ ID NO: 239) | | | | | |
| SP599.CD5.g46 | CCGTGGCTCCCCTGGGTCTTNGG | 1 | 2 | 5 | 37 | rs2241002: 0.158 |
| | (SEQ ID NO: 240) | | | | | |
| SP599.CD5.g28 | AAAATCAAGCCCCAGAAAAGNGG | 1 | 2 | 5 | 60 | NA |
| | (SEQ ID NO: 241) | | | | | |
| SP599.CD5.g18 | GAAGCATCTGCCAGAGACTGNGG | 1 | 2 | 7 | 98 | NA |
| | (SEQ ID NO: 242) | | | | | |
| SP599.CD5.g24 | GAAGCATCTGCCAGAGACTGNGG | 1 | 2 | 8 | 56 | rs2241002: 0.158 |
| | (SEQ ID NO: 243) | | | | | |
| SP599.CD5.g22 | AGGCAGAGCCCAAGACCCAGNGG | 1 | 2 | 10 | 41 | rs2241002: 0.158 |
| | (SEQ ID NO: 244) | | | | | |
| SP599.CD5.g23 | CCCAAGACCCAGGGGAGCCANGG | 1 | 2 | 10 | 99 | rs2241002: 0.158 |
| | (SEQ ID NO: 245) | | | | | |

The gRNA sequences in Table 17, Table 18, and Table 19 were normalized (% Normalization to NHEJ) for gRNA activity via next generation sequencing (NGS). GFP was used as a control. Following sequencing analysis, the following gRNAs were recommended based on off-target profile: Exon 3: SP597.hCD5.g2 (76.5%), SP597.hCD5.g22 (36.3%), SP597.hCD5.g39 (16.0%), SP597.hCD5.g46. Exon4: SP598.hCD5.g7, SP598.hCD5.g10 (58.5%). Exon5: SP599.hCD5.g5 (51.0%), SP599.hCD5.g30, SP599.hCD5.g42, SP599.hCD5.g58 (41.0%)

Off target analysis of selected gRNA was performed for hCSF2 to determine the number of sites in human genome which are an exact match or contains up to 1 or 2 mismatches, which may include the target site. The results are listed in Table 20 for hCSF2.

TABLE 20
Guide RNA (gRNA) Off Target Analysis for hCSF2

| Name | gRNA | long_0 | long_1 | long_2 | long_3 | short_0 | SNP |
|---|---|---|---|---|---|---|---|
| MS1086.CSF2.sp8 | TACTCAGGTTCAGGAGACGCNGG | 1 | 1 | 1 | 10 | 11 | NA |
| | (SEQ ID NO: 246) | | | | | | |
| MS1086.CSF2.sp10 | TCAGGAGACGCCGGGCCTCCNGG | 1 | 1 | 1 | 20 | 38 | NA |
| | (SEQ ID NO: 247) | | | | | | |
| MS1086.CSF2.sp9 | ACTCAGGTTCAGGAGACGCCNGG | 1 | 1 | 1 | 20 | 16 | NA |
| | (SEQ ID NO: 248) | | | | | | |
| MS1086.CSF2.sp7 | CAGTGTCTCTACTCAGGTTCNGG | 1 | 1 | 2 | 22 | 29 | NA |
| | (SEQ ID NO: 249) | | | | | | |
| MS1086.CSF2.sp14 | ATGCTCCCAGGGCTGCGTGCNGG | 1 | 1 | 2 | 42 | 34 | rs2069622 |
| | (SEQ ID NO: 250) | | | | | | |
| MS1086.CSF2.sp11 | GAGACGCCGGGCCTCCTGGANGG | 1 | 1 | 2 | 26 | 146 | NA |
| | (SEQ ID NO: 251) | | | | | | |
| MS1086.CSF2.sp6 | CAGCAGCAGTGTCTCTACTCNGG | 1 | 1 | 3 | 39 | 24 | NA |
| | (SEQ ID NO: 252) | | | | | | |
| MS1086.CSF2.sp12 | GATGGCATTCACATGCTCCCNGG | 1 | 1 | 3 | 28 | 59 | NA |
| | (SEQ ID NO: 253) | | | | | | |
| MS1086.CSF2.sp2 | GGAGCATGTGAATGCCATCCNGG | 1 | 1 | 3 | 26 | 48 | NA |
| | (SEQ ID NO: 254) | | | | | | |
| MS1086.CSF2.sp5 | TAGAGACACTGCTGCTGAGANGG | 1 | 1 | 3 | 56 | 168 | NA |
| | (SEQ ID NO: 255) | | | | | | |
| MS1086.CSF2.sp3 | GCATGTGAATGCCATCCAGGNGG | 1 | 1 | 3 | 41 | 56 | NA |
| | (SEQ ID NO: 256) | | | | | | |
| MS1086.CSF2.sp13 | ATGGCATTCACATGCTCCCANGG | 1 | 1 | 4 | 30 | 80 | NA |
| | (SEQ ID NO: 257) | | | | | | |
| MS1086.CSF2.sp4 | TGAATGCCATCCAGGAGGCCNGG | 1 | 1 | 5 | 65 | 180 | NA |
| | (SEQ ID NO: 258) | | | | | | |
| MS1086.CSF2.sp15 | TGCTCCCAGGGCTGCGTGCTNGG | 1 | 1 | 6 | 57 | 29 | rs2069622 |
| | (SEQ ID NO: 259) | | | | | | |
| MS1086.CSF2.sp1 | CAGCCCCAGCACGCAGCCCTNGG | 1 | 1 | 15 | 146 | 41 | rs2069622 |
| | (SEQ ID NO: 260) | | | | | | |
| MS1086.CSF2.sp16 | GCTCCCAGGGCTGCGTGCTGNGG | 1 | 2 | 9 | 85 | 37 | rs2069622 |
| | (SEQ ID NO: 261) | | | | | | |

The gRNA sequences in Table 20 were normalized (% Normalization to NHEJ) for gRNA activity via next generation sequencing (NGS). GFP was used as a control. Following sequencing analysis, the following gRNAs were recommended based on off-target profile: MS1086.CSF2.sp8 (>15%) and MS1086.CSF2.sp10 (>15%).

Off target analysis of selected gRNA was performed for 2 exons of hCTLA4 (Exon 1 and Exon 2) to determine the number of sites in human genome which are an exact match or contains up to 1 or 2 mismatches, which may include the target site. The results are listed in Table 21 for Exon 1 and Table 22 for Exon 2 for hCTLA4.

TABLE 21
Guide RNA (gRNA) Off Target Analysis for hCTLA4 (Exon 1)

| Name | gRNA | long_0 | long_1 | long_2 | short_0 | SNP |
|---|---|---|---|---|---|---|
| SP621.CTLA4.g2 | CCTTGGATTTCAGCGGCACANGG | 1 | 1 | 1 | 5 | NA |
| | (SEQ ID NO: 262) | | | | | |
| SP621.CTLA4.g12 | CCTTGTGCCGCTGAAATCCANGG | 1 | 1 | 1 | 5 | NA |
| | (SEQ ID NO: 263) | | | | | |
| SP621.CTLA4.g5 | TGAACCTGGCTACCAGGACCNGG | 1 | 1 | 1 | 11 | rs231775: 0.452 |
| | (SEQ ID NO: 264) | | | | | |
| SP621.CTLA4.g11 | AGGGCCAGGTCCTGGTAGCCNGG | 1 | 1 | 3 | 16 | rs231775: 0.452 |
| | (SEQ ID NO: 265) | | | | | |
| SP621.CTLA4.g4 | CTCAGCTGAACCTGGCTACCNGG | 1 | 1 | 3 | 17 | rs231775: 0.452 |
| | (SEQ ID NO: 266) | | | | | |
| SP621.CTLA4.g8 | AGAAAAAACAGGAGAGTGCANGG | 1 | 1 | 3 | 39 | NA |
| | (SEQ ID NO: 267) | | | | | |
| SP621.CTLA4.g3 | GCACAAGGCTCAGCTGAACCNGG | 1 | 1 | 4 | 29 | NA |
| | (SEQ ID NO: 268) | | | | | |
| SP621.CTLA4.g1 | TGGCTTGCCTTGGATTTCAGNGG | 1 | 1 | 6 | 33 | NA |
| | (SEQ ID NO: 269) | | | | | |
| SP621.CTLA4.g9 | AAACAGGAGAGTGCAGGGCCNGG | 1 | 1 | 6 | 69 | NA |
| | (SEQ ID NO: 270) | | | | | |
| SP621.CTLA4.g10 | GAGAGTGCAGGGCCAGGTCCNGG | 1 | 1 | 7 | 50 | NA |
| | (SEQ ID NO: 271) | | | | | |
| SP621.CTLA4.g6 | GGATGAAGAGAAGAAAAAACNGG | 1 | 1 | 8 | 173 | NA |
| | (SEQ ID NO: 272) | | | | | |
| SP621.CTLA4.g7 | AAGAAAAAACAGGAGAGTGCNGG | 1 | 2 | 8 | 33 | NA |
| | (SEQ ID NO: 273) | | | | | |

TABLE 22
Guide RNA (gRNA) Off Target Analysis for hCTLA4 (Exon 2)

| Name | gRNA | long_0 | long_1 | long_2 | short_0 | SNP |
|---|---|---|---|---|---|---|
| SP622.CTLA4.g9 | CCGGGTGACAGTGCTTCGGCNGG | 1 | 1 | 1 | 2 | NA |
| | (SEQ ID NO: 274) | | | | | |
| SP622.CTLA4.g33 | ACACAAAGCTGGCGATGCCTNGG | 1 | 1 | 1 | 4 | NA |
| | (SEQ ID NO: 275) | | | | | |
| SP622.CTLA4.g21 | CCCTCAGTCCTTGGATAGTGNGG | 1 | 1 | 1 | 8 | NA |
| | (SEQ ID NO: 276) | | | | | |
| SP622.CTLA4.g14 | GTGCGGCAACCTACATGATGNGG | 1 | 1 | 1 | 9 | NA |
| | (SEQ ID NO: 277) | | | | | |
| SP622.CTLA4.g12 | CTGTGCGGCAACCTACATGANGG | 1 | 1 | 1 | 13 | NA |
| | (SEQ ID NO: 278) | | | | | |
| SP622.CTLA4.g2 | GGCCCAGCCTGCTGTGGTACNGG | 1 | 1 | 1 | 17 | NA |
| | (SEQ ID NO: 279) | | | | | |
| SP622.CTLA4.g23 | GTTCACTTGATTTCCACTGGNGG | 1 | 1 | 1 | 17 | NA |
| | (SEQ ID NO: 280) | | | | | |
| SP622.CTLA4.g27 | CAACTCATTCCCCATCATGTNGG | 1 | 1 | 1 | 18 | NA |
| | (SEQ ID NO: 281) | | | | | |
| SP622.CTLA4.g28 | CCGCACAGACTTCAGTCACCNGG | 1 | 1 | 1 | 20 | NA |
| | (SEQ ID NO: 282) | | | | | |
| SP622.CTLA4.g13 | TGTGCGGCAACCTACATGATNGG | 1 | 1 | 1 | 30 | NA |
| | (SEQ ID NO: 283) | | | | | |
| SP622.CTLA4.g20 | CCTCACTATCCAAGGACTGANGG | 1 | 1 | 1 | 30 | NA |
| | (SEQ ID NO: 284) | | | | | |
| SP622.CTLA4.g31 | CGGACCTCAGTGGCTTTGCCNGG | 1 | 1 | 1 | 34 | NA |
| | (SEQ ID NO: 285) | | | | | |
| SP622.CTLA4.g22 | GAGGTTCACTTGATTTCCACNGG | 1 | 1 | 1 | 40 | NA |
| | (SEQ ID NO: 286) | | | | | |
| SP622.CTLA4.g11 | CCAGGTGACTGAAGTCTGTGNGG | 1 | 1 | 1 | 45 | NA |
| | (SEQ ID NO: 287) | | | | | |
| SP622.CTLA4.g24 | ACTGGAGGTGCCCGTGCAGANGG | 1 | 1 | 2 | 15 | NA |
| | (SEQ ID NO: 288) | | | | | |
| SP622.CTLA4.g18 | CAAGTGAACCTCACTATCCANGG | 1 | 1 | 2 | 16 | NA |
| | (SEQ ID NO: 289) | | | | | |
| SP622.CTLA4.g3 | GTGGTACTGGCCAGCAGCCGNGG | 1 | 1 | 2 | 29 | NA |
| | (SEQ ID NO: 290) | | | | | |
| SP622.CTLA4.g8 | AGGTCCGGGTGACAGTGCTTNGG | 1 | 1 | 2 | 29 | NA |
| | (SEQ ID NO: 291) | | | | | |
| SP622.CTLA4.g17 | ATCTGCACGGGCACCTCCAGNGG | 1 | 1 | 2 | 29 | NA |
| | (SEQ ID NO: 292) | | | | | |
| SP622.CTLA4.g25 | CCGTGCAGATGGAATCATCTNGG | 1 | 1 | 2 | 36 | NA |
| | (SEQ ID NO: 293) | | | | | |
| SP622.CTLA4.g16 | CTAGATGATTCCATCTGCACNGG | 1 | 1 | 2 | 39 | NA |
| | (SEQ ID NO: 294) | | | | | |
| SP622.CTLA4.g19 | ACCTCACTATCCAAGGACTGNGG | 1 | 1 | 2 | 40 | NA |
| | (SEQ ID NO: 295) | | | | | |
| SP622.CTLA4.g29 | CCTGCCGAAGCACTGTCACCNGG | 1 | 1 | 2 | 47 | NA |
| | (SEQ ID NO: 296) | | | | | |
| SP622.CTLA4.g36 | TGGCCAGTACCACAGCAGGCNGG | 1 | 1 | 2 | 74 | NA |
| | (SEQ ID NO: 297) | | | | | |
| SP622.CTLA4.g5 | ATCTCCAGGCAAAGCCACTGNGG | 1 | 1 | 2 | 80 | NA |
| | (SEQ ID NO: 298) | | | | | |
| SP622.CTLA4.g1 | GCACGTGGCCCAGCCTGCTGNGG | 1 | 1 | 2 | 121 | NA |
| | (SEQ ID NO: 299) | | | | | |
| SP622.CTLA4.g4 | GTGTGTGAGTATGCATCTCCNGG | 1 | 1 | 3 | 8 | NA |
| | (SEQ ID NO: 300) | | | | | |
| SP622.CTLA4.g30 | CACTGTCACCCGGACCTCAGNGG | 1 | 1 | 3 | 9 | NA |
| | (SEQ ID NO: 301) | | | | | |
| SP622.CTLA4.g34 | GCTGGCGATGCCTCGGCTGCNGG | 1 | 1 | 3 | 17 | NA |
| | (SEQ ID NO: 302) | | | | | |
| SP622.CTLA4.g35 | CTGCTGGCCAGTACCACAGCNGG | 1 | 1 | 3 | 22 | NA |
| | (SEQ ID NO: 303) | | | | | |
| SP622.CTLA4.g7 | AGGCAAAGCCACTGAGGTCCNGG | 1 | 1 | 3 | 40 | NA |
| | (SEQ ID NO: 304) | | | | | |
| SP622.CTLA4.g26 | GCAGATGGAATCATCTAGGANGG | 1 | 1 | 4 | 20 | NA |
| | (SEQ ID NO: 305) | | | | | |
| SP622.CTLA4.g15 | CCTAGATGATTCCATCTGCANGG | 1 | 1 | 4 | 40 | NA |
| | (SEQ ID NO: 306) | | | | | |
| SP622.CTLA4.g37 | GGCCAGTACCACAGCAGGCTNGG | 1 | 1 | 4 | 65 | NA |
| | (SEQ ID NO: 307) | | | | | |
| SP622.CTLA4.g32 | TGCATACTCACACACAAAGCNGG | 1 | 1 | 7 | 71 | NA |
| | (SEQ ID NO: 308) | | | | | |
| SP622.CTLA4.g10 | GCTTCGGCAGGCTGACAGCCNGG | 1 | 1 | 8 | 58 | NA |
| | (SEQ ID NO: 309) | | | | | |
| SP622.CTLA4.g6 | CAGGCAAAGCCACTGAGGTCNGG | 1 | 1 | 11 | 30 | NA |
| | (SEQ ID NO: 310) | | | | | |

The gRNA sequences in Table 21 and Table 22 were normalized (% Normalization to NHEJ) for gRNA activity via next generation sequencing (NGS). GFP was used as a control. Following sequencing analysis, the following gRNAs were recommended based on off-target profile: Exon 1: SP621.hCTLA4.g2 (>15%) and SP621.hCTLA4.g12 (>15%). Exon 2: SP622.hCTLA4.g2 (>15%), SP622.hCTLA4.g9 (>15%), and SP622.hCTLA4.g33 (>15%).

Off target analysis of selected gRNA was performed for 2 exons of hPDCD1 (CF60 and CF61) to determine the number of sites in human genome which are an exact match or contains up to 1 or 2 mismatches, which may include the target site. The results are listed in Table 23 for Exon CF60 and Table 24 for Exon CF61.

TABLE 23
Guide RNA (gRNA) Off Target Analysis for hPDCD1 (Exon CF60)

| Name | gRNA | long_0 | long_1 | long_2 | short_0 | SNP |
|---|---|---|---|---|---|---|
| CF60.PDCD1.g12 | TGTAGCACCGCCCAGACGACNGG | 1 | 1 | 1 | 1 | NA |
| | (SEQ ID NO: 311) | | | | | |
| CF60.PDCD1.g3 | GGCGCCCTGGCCAGTCGTCTNGG | 1 | 1 | 1 | 3 | NA |
| | (SEQ ID NO: 312) | | | | | |
| CF60.PDCD1.g5 | CGTCTGGGCGGTGCTACAACNGG | 1 | 1 | 1 | 3 | NA |
| | (SEQ ID NO: 313) | | | | | |
| CF60.PDCD1.g2 | AGGCGCCCTGGCCAGTCGTCNGG | 1 | 1 | 1 | 5 | NA |
| | (SEQ ID NO: 314) | | | | | |
| CF60.PDCD1.g13 | CACCGCCCAGACGACTGGCCNGG | 1 | 1 | 1 | 5 | NA |
| | (SEQ ID NO: 315) | | | | | |
| CF60.PDCD1.g14 | ACCGCCCAGACGACTGGCCANGG | 1 | 1 | 1 | 5 | NA |
| | (SEQ ID NO: 316) | | | | | |
| CF60.PDCD1.g7 | GGGCGGTGCTACAACTGGGCNGG | 1 | 1 | 1 | 7 | NA |
| | (SEQ ID NO: 317) | | | | | |
| CF60.PDCD1.g6 | GTCTGGGCGGTGCTACAACTNGG | 1 | 1 | 1 | 9 | NA |
| | (SEQ ID NO: 318) | | | | | |
| CF60.PDCD1.g16 | CGACTGGCCAGGGCGCCTGTNGG | 1 | 1 | 1 | 15 | NA |
| | (SEQ ID NO: 319) | | | | | |
| CF60.PDCD1.g8 | CGGTGCTACAACTGGGCTGGNGG | 1 | 1 | 1 | 33 | NA |
| | (SEQ ID NO: 320) | | | | | |
| CF60.PDCD1.g11 | TGGCGGCCAGGATGGTTCTTNGG | 1 | 1 | 1 | 33 | NA |
| | (SEQ ID NO: 321) | | | | | |
| CF60.PDCD1.g15 | ACGACTGGCCAGGGCGCCTGNGG | 1 | 1 | 1 | 45 | NA |
| | (SEQ ID NO: 322) | | | | | |
| CF60.PDCD1.g9 | CTACAACTGGGCTGGCGGCCNGG | 1 | 1 | 1 | 57 | NA |
| | (SEQ ID NO: 323) | | | | | |
| CF60.PDCD1.g4 | GCCCTGGCCAGTCGTCTGGGNGG | 1 | 1 | 2 | 2 | NA |
| | (SEQ ID NO: 324) | | | | | |
| CF60.PDCD1.g1 | TGCAGATCCCACAGGCGCCCNGG | 1 | 1 | 2 | 23 | NA |
| | (SEQ ID NO: 325) | | | | | |
| CF60.PDCD1.g10 | AACTGGGCTGGCGGCCAGGANGG | 1 | 1 | 3 | 17 | NA |
| | (SEQ ID NO: 326) | | | | | |

TABLE 24
Guide RNA (gRNA) Off Target Analysis for hPDCD1 (CF61)

| Name | gRNA | long_0 | long_1 | long_2 | short_0 | SNP |
|---|---|---|---|---|---|---|
| CF61.PDCD1.g6 | CGGAGAGCTTCGTGCTAAACNGG | 1 | 1 | 1 | 1 | NA |
| | (SEQ ID NO: 327) | | | | | |
| CF61.PDCD1.g14 | GCGTGACTTCCACATGAGCGNGG | 1 | 1 | 1 | 2 | NA |
| | (SEQ ID NO: 328) | | | | | |
| CF61.PDCD1.g17 | ATGTGGAAGTCACGCCCGTTNGG | 1 | 1 | 1 | 2 | NA |
| | (SEQ ID NO: 329) | | | | | |
| CF61.PDCD1.g2 | GCCCTGCTCGTGGTGACCGANGG | 1 | 1 | 1 | 3 | NA |
| | (SEQ ID NO: 330) | | | | | |
| CF61.PDCD1.g35 | CACGAAGCTCTCCGATGTGTNGG | 1 | 1 | 1 | 3 | NA |
| | (SEQ ID NO: 331) | | | | | |
| CF61.PDCD1.g4 | CCTGCTCGTGGTGACCGAAGNGG | 1 | 1 | 1 | 4 | NA |
| | (SEQ ID NO: 332) | | | | | |
| CF61.PDCD1.g20 | TGACACGGAAGCGGCAGTCCNGG | 1 | 1 | 1 | 5 | NA |
| | (SEQ ID NO: 333) | | | | | |
| CF61.PDCD1.g40 | CCCCTTCGGTCACCACGAGCNGG | 1 | 1 | 1 | 5 | NA |
| | (SEQ ID NO: 334) | | | | | |
| CF61.PDCD1.g8 | CAGCAACCAGACGGACAAGCNGG | 1 | 1 | 1 | 6 | NA |
| | (SEQ ID NO: 335) | | | | | |
| CF61.PDCD1.g19 | GCAGTTGTGTGACACGGAAGNGG | 1 | 1 | 1 | 6 | NA |
| | (SEQ ID NO: 336) | | | | | |
| CF61.PDCD1.g41 | CCCTTCGGTCACCACGAGCANGG | 1 | 1 | 1 | 6 | NA |
| | (SEQ ID NO: 337) | | | | | |
| CF61.PDCD1.g26 | CCGGGCTGGCTGCGGTCCTCNGG | 1 | 1 | 1 | 8 | NA |
| | (SEQ ID NO: 338) | | | | | |
| CF61.PDCD1.g30 | AGGCGGCCAGCTTGTCCGTCNGG | 1 | 1 | 1 | 8 | NA |
| | (SEQ ID NO: 339) | | | | | |
| CF61.PDCD1.g31 | CAGCTTGTCCGTCTGGTTGCNGG | 1 | 1 | 1 | 8 | NA |
| | (SEQ ID NO: 340) | | | | | |
| CF61.PDCD1.g43 | CGGTCACCACGAGCAGGGCTNGG | 1 | 1 | 1 | 10 | NA |
| | (SEQ ID NO: 341) | | | | | |
| CF61.PDCD1.g13 | GTGTCACACAACTGCCCAACNGG | 1 | 1 | 1 | 13 | NA |
| | (SEQ ID NO: 342) | | | | | |
| CF61.PDCD1.g5 | CTGCAGCTTCTCCAACACATNGG | 1 | 1 | 1 | 23 | NA |
| | (SEQ ID NO: 343) | | | | | |
| CF61.PDCD1.g9 | CAAGCTGGCCGCCTTCCCCGNGG | 1 | 1 | 1 | 23 | NA |
| | (SEQ ID NO: 344) | | | | | |
| CF61.PDCD1.g12 | CGTGTCACACAACTGCCCAANGG | 1 | 1 | 1 | 28 | NA |
| | (SEQ ID NO: 345) | | | | | |
| CF61.PDCD1.g18 | CGTTGGGCAGTTGTGTGACANGG | 1 | 1 | 1 | 32 | NA |
| | (SEQ ID NO: 346) | | | | | |
| CF61.PDCD1.g33 | GCTTGTCCGTCTGGTTGCTGNGG | 1 | 1 | 1 | 41 | NA |
| | (SEQ ID NO: 347) | | | | | |
| CF61.PDCD1.g22 | CGGAAGCGGCAGTCCTGGCCNGG | 1 | 1 | 1 | 61 | NA |
| | (SEQ ID NO: 348) | | | | | |
| CF61.PDCD1.g36 | CGATGTGTTGGAGAAGCTGCNGG | 1 | 1 | 1 | 135 | NA |
| | (SEQ ID NO: 349) | | | | | |
| CF61.PDCD1.g16 | CATGTGGAAGTCACGCCCGTNGG | 1 | 1 | 2 | 2 | NA |
| | (SEQ ID NO: 350) | | | | | |
| CF61.PDCD1.g3 | CCCTGCTCGTGGTGACCGAANGG | 1 | 1 | 2 | 3 | NA |
| | (SEQ ID NO: 351) | | | | | |
| CF61.PDCD1.g27 | CGGGCTGGCTGCGGTCCTCGNGG | 1 | 1 | 2 | 3 | NA |
| | (SEQ ID NO: 352) | | | | | |
| CF61.PDCD1.g32 | AGCTTGTCCGTCTGGTTGCTNGG | 1 | 1 | 2 | 4 | NA |
| | (SEQ ID NO: 353) | | | | | |
| CF61.PDCD1.g39 | GAAGGTGGCGTTGTCCCCTTNGG | 1 | 1 | 2 | 4 | NA |
| | (SEQ ID NO: 354) | | | | | |
| CF61.PDCD1.g15 | ACTTCCACATGAGCGTGGTCNGG | 1 | 1 | 2 | 6 | NA |
| | (SEQ ID NO: 355) | | | | | |
| CF61.PDCD1.g25 | GCCGGGCTGGCTGCGGTCCTNGG | 1 | 1 | 2 | 17 | NA |
| | (SEQ ID NO: 356) | | | | | |
| CF61.PDCD1.g42 | TCGGTCACCACGAGCAGGGCNGG | 1 | 1 | 2 | 23 | NA |
| | (SEQ ID NO: 357) | | | | | |
| CF61.PDCD1.g34 | TCTGGTTGCTGGGGCTCATGNGG | 1 | 1 | 2 | 31 | NA |
| | (SEQ ID NO: 358) | | | | | |
| CF61.PDCD1.g21 | ACGGAAGCGGCAGTCCTGGCNGG | 1 | 1 | 2 | 41 | NA |
| | (SEQ ID NO: 359) | | | | | |
| CF61.PDCD1.g10 | CCCGAGGACCGCAGCCAGCCNGG | 1 | 1 | 2 | 46 | NA |
| | (SEQ ID NO: 360) | | | | | |
| CF61.PDCD1.g28 | CTGGCTGCGGTCCTCGGGGANGG | 1 | 1 | 3 | 16 | NA |
| | (SEQ ID NO: 361) | | | | | |
| CF61.PDCD1.g7 | CATGAGCCCCAGCAACCAGANGG | 1 | 1 | 3 | 33 | NA |
| | (SEQ ID NO: 362) | | | | | |
| CF61.PDCD1.g24 | AGTCCTGGCCGGGCTGGCTGNGG | 1 | 1 | 3 | 42 | NA |
| | (SEQ ID NO: 363) | | | | | |
| CF61.PDCD1.g55 | GGGGGTTCCAGGGCCTGTCTNGG | 1 | 1 | 3 | 126 | NA |
| | (SEQ ID NO: 364) | | | | | |
| CF61.PDCD1.g44 | GGTCACCACGAGCAGGGCTGNGG | 1 | 1 | 4 | 26 | NA |
| | (SEQ ID NO: 365) | | | | | |
| CF61.PDCD1.g29 | GCTGCGGTCCTCGGGGAAGGNGG | 1 | 1 | 4 | 35 | NA |
| | (SEQ ID NO: 366) | | | | | |
| CF61.PDCD1.g11 | GGACCGCAGCCAGCCCGGCCNGG | 1 | 1 | 4 | 47 | NA |
| | (SEQ ID NO: 367) | | | | | |
| CF61.PDCD1.g53 | GAGAAGGTGGGGGGGTTCCANGG | 1 | 1 | 5 | 8 | NA |
| | (SEQ ID NO: 368) | | | | | |
| CF61.PDCD1.g52 | GGAGAAGGTGGGGGGGTTCCNGG | 1 | 1 | 5 | 15 | NA |
| | (SEQ ID NO: 369) | | | | | |
| CF61.PDCD1.g23 | AGCGGCAGTCCTGGCCGGGCNGG | 1 | 1 | 5 | 39 | NA |
| | (SEQ ID NO: 370) | | | | | |
| CF61.PDCD1.g56 | GGGGTTCCAGGGCCTGTCTGNGG | 1 | 1 | 5 | 97 | NA |
| | (SEQ ID NO: 371) | | | | | |
| CF61.PDCD1.g1 | CTTCTCCCCAGCCCTGCTCGNGG | 1 | 1 | 6 | 22 | NA |
| | (SEQ ID NO: 372) | | | | | |
| CF61.PDCD1.g37 | GTTGGAGAAGCTGCAGGTGANGG | 1 | 1 | 6 | 88 | NA |
| | (SEQ ID NO: 373) | | | | | |
| CF61.PDCD1.g54 | GGGGGGTTCCAGGGCCTGTCNGG | 1 | 1 | 6 | 1286 | NA |
| | (SEQ ID NO: 374) | | | | | |
| CF61.PDCD1.g38 | GGAGAAGCTGCAGGTGAAGGNGG | 1 | 1 | 9 | 66 | NA |
| | (SEQ ID NO: 375) | | | | | |
| CF61.PDCD1.g45 | CACGAGCAGGGCTGGGGAGANGG | 1 | 1 | 10 | 448 | NA |
| | (SEQ ID NO: 376) | | | | | |
| CF61.PDCD1.g48 | GCAGGGCTGGGGAGAAGGTGNGG | 1 | 1 | 21 | 125 | NA |
| | (SEQ ID NO: 377) | | | | | |
| CF61.PDCD1.g49 | CAGGGCTGGGGAGAAGGTGGNGG | 1 | 1 | 29 | 214 | NA |
| | (SEQ ID NO: 378) | | | | | |
| CF61.PDCD1.g46 | GAGCAGGGCTGGGGAGAAGGNGG | 1 | 1 | 30 | 202 | NA |
| | (SEQ ID NO: 379) | | | | | |
| CF61.PDCD1.g47 | AGCAGGGCTGGGGAGAAGGTNGG | 1 | 2 | 11 | 136 | NA |
| | (SEQ ID NO: 380) | | | | | |
| CF61.PDCD1.g50 | AGGGCTGGGGAGAAGGTGGGNGG | 1 | 2 | 31 | 179 | NA |
| | (SEQ ID NO: 381) | | | | | |
| CF61.PDCD1.g51 | GGGCTGGGGAGAAGGTGGGGNGG | 1 | 2 | 49 | 130 | NA |
| | (SEQ ID NO: 382) | | | | | |

The gRNA sequences in Table 23 and Table 24 were normalized (% Normalization to NHEJ) for gRNA activity via next generation sequencing (NGS). GFP was used as a control. Following sequencing analysis, the following gRNAs were recommended based on off-target profile: CF60.PDCD1.g12 (65.6%), CF60.PDCD1.g3 (69.2%), CF61.PDCD1.g6, CF61.PDCD1.g2 (72.7%), and CF61.PDCD1.g35 (24.0%).

Off target analysis of selected gRNA was performed for 2 exons of hTIM3 (Exon 2 and Exon 3) to determine the number of sites in human genome which are an exact match or contains up to 1 or 2 mismatches, which may include the target site. The results are listed in Table 25 for Exon 2 and Table 26 for Exon 3.

TABLE 25
Guide RNA (gRNA) Off Target Analysis for hTIM3 (Exon 2)

| Name | gRNA | long_0 | long_1 | long_2 | short_0 | SNP |
|---|---|---|---|---|---|---|
| SP619.TIM3.g2 | AGAAGTGGAATACAGAGCGGNGG | 1 | 1 | 1 | 2 | NA |
| | (SEQ ID NO: 383) | | | | | |
| SP619.TIM3.g12 | AATGTGGCAACGTGGTGCTCNGG | 1 | 1 | 1 | 3 | NA |
| | (SEQ ID NO: 384) | | | | | |
| SP619.TIM3.g20 | CTAAATGGGGATTTCCGCAANGG | 1 | 1 | 1 | 4 | NA |
| | (SEQ ID NO: 385) | | | | | |
| SP619.TIM3.g18 | CATCCAGATACTGGCTAAATNGG | 1 | 1 | 1 | 8 | NA |
| | (SEQ ID NO: 386) | | | | | |
| SP619.TIM3.g41 | CAGACGGGCACGAGGTTCCCNGG | 1 | 1 | 1 | 8 | NA |
| | (SEQ ID NO: 387) | | | | | |
| SP619.TIM3.g49 | GCGGCTGGGGTGTAGAAGCANGG | 1 | 1 | 1 | 8 | NA |
| | (SEQ ID NO: 388) | | | | | |
| SP619.TIM3.g7 | GAACCTCGTGCCCGTCTGCTNGG | 1 | 1 | 1 | 10 | NA |
| | (SEQ ID NO: 389) | | | | | |
| SP619.TIM3.g43 | GACGGGCACGAGGTTCCCTGNGG | 1 | 1 | 1 | 10 | NA |
| | (SEQ ID NO: 390) | | | | | |
| SP619.TIM3.g35 | ATCCCCATTTAGCCAGTATCNGG | 1 | 1 | 1 | 11 | NA |
| | (SEQ ID NO: 391) | | | | | |
| SP619.TIM3.g3 | GTGGAATACAGAGCGGAGGTNGG | 1 | 1 | 1 | 12 | NA |
| | (SEQ ID NO: 392) | | | | | |
| SP619.TIM3.g42 | AGACGGGCACGAGGTTCCCTNGG | 1 | 1 | 1 | 12 | NA |
| | (SEQ ID NO: 393) | | | | | |
| SP619.TIM3.g6 | GGAACCTCGTGCCCGTCTGCNGG | 1 | 1 | 1 | 13 | NA |
| | (SEQ ID NO: 394) | | | | | |
| SP619.TIM3.g32 | GAGTCACATTCTCTATGGTCNGG | 1 | 1 | 1 | 14 | NA |
| | (SEQ ID NO: 395) | | | | | |
| SP619.TIM3.g22 | ATGTGACTCTAGCAGACAGTNGG | 1 | 1 | 1 | 16 | NA |
| | (SEQ ID NO: 396) | | | | | |
| SP619.TIM3.g27 | TTTTCATCATTCATTATGCCNGG | 1 | 1 | 1 | 16 | NA |
| | (SEQ ID NO: 397) | | | | | |
| SP619.TIM3.g21 | AATGTGACTCTAGCAGACAGNGG | 1 | 1 | 1 | 17 | NA |
| | (SEQ ID NO: 398) | | | | | |
| SP619.TIM3.g19 | ATCCAGATACTGGCTAAATGNGG | 1 | 1 | 1 | 18 | NA |
| | (SEQ ID NO: 399) | | | | | |
| SP619.TIM3.g24 | TGCTGCCGGATCCAAATCCCNGG | 1 | 1 | 1 | 22 | NA |
| | (SEQ ID NO: 400) | | | | | |
| SP619.TIM3.g5 | TCTACACCCCAGCCGCCCCANGG | 1 | 1 | 1 | 30 | NA |
| | (SEQ ID NO: 401) | | | | | |
| SP619.TIM3.g30 | TTATGCCTGGGATTTGGATCNGG | 1 | 1 | 1 | 35 | NA |
| | (SEQ ID NO: 402) | | | | | |
| SP619.TIM3.g51 | CGCTCTGTATTCCACTTCTGNGG | 1 | 1 | 1 | 83 | NA |
| | (SEQ ID NO: 403) | | | | | |
| SP619.TIM3.g47 | GAGGTTCCCTGGGGCGGCTGNGG | 1 | 1 | 1 | 85 | NA |
| | (SEQ ID NO: 404) | | | | | |
| SP619.TIM3.g40 | TGCCCCAGCAGACGGGCACGNGG | 1 | 1 | 2 | 5 | NA |
| | (SEQ ID NO: 405) | | | | | |
| SP619.TIM3.g23 | ACAGTGGGATCTACTGCTGCNGG | 1 | 1 | 2 | 8 | NA |
| | (SEQ ID NO: 406) | | | | | |
| SP619.TIM3.g11 | TGTGTTTGAATGTGGCAACGNGG | 1 | 1 | 2 | 9 | NA |
| | (SEQ ID NO: 407) | | | | | |
| SP619.TIM3.g25 | TGAAAAATTTAACCTGAAGTNGG | 1 | 1 | 2 | 16 | NA |
| | (SEQ ID NO: 408) | | | | | |
| SP619.TIM3.g17 | ACATCCAGATACTGGCTAAANGG | 1 | 1 | 2 | 19 | NA |
| | (SEQ ID NO: 409) | | | | | |
| SP619.TIM3.g15 | ATGAAAGGGATGTGAATTATNGG | 1 | 1 | 2 | 22 | NA |
| | (SEQ ID NO: 410) | | | | | |
| SP619.TIM3.g13 | TGGTGCTCAGGACTGATGAANGG | 1 | 1 | 2 | 25 | NA |
| | (SEQ ID NO: 411) | | | | | |
| SP619.TIM3.g50 | GGTGTAGAAGCAGGGCAGATNGG | 1 | 1 | 2 | 36 | NA |
| | (SEQ ID NO: 412) | | | | | |
| SP619.TIM3.g36 | ACGTTGCCACATTCAAACACNGG | 1 | 1 | 2 | 37 | NA |
| | (SEQ ID NO: 413) | | | | | |
| SP619.TIM3.g45 | ACGAGGTTCCCTGGGGCGGCNGG | 1 | 1 | 2 | 40 | NA |
| | (SEQ ID NO: 414) | | | | | |
| SP619.TIM3.g10 | GCCTGTCCTGTGTTTGAATGNGG | 1 | 1 | 2 | 47 | NA |
| | (SEQ ID NO: 415) | | | | | |
| SP619.TIM3.g9 | GTGCCCGTCTGCTGGGGCAANGG | 1 | 1 | 2 | 58 | NA |
| | (SEQ ID NO: 416) | | | | | |
| SP619.TIM3.g8 | AACCTCGTGCCCGTCTGCTGNGG | 1 | 1 | 3 | 15 | NA |
| | (SEQ ID NO: 417) | | | | | |
| SP619.TIM3.g48 | GGCGGCTGGGGTGTAGAAGCNGG | 1 | 1 | 3 | 15 | NA |
| | (SEQ ID NO: 418) | | | | | |
| SP619.TIM3.g33 | AGTCACATTCTCTATGGTCANGG | 1 | 1 | 3 | 19 | NA |
| | (SEQ ID NO: 419) | | | | | |
| SP619.TIM3.g26 | CTGGTTTGATGACCAACTTCNGG | 1 | 1 | 3 | 21 | NA |
| | (SEQ ID NO: 420) | | | | | |
| SP619.TIM3.g29 | CATTCATTATGCCTGGGATTNGG | 1 | 1 | 3 | 24 | NA |
| | (SEQ ID NO: 421) | | | | | |
| SP619.TIM3.g31 | TGCTAGAGTCACATTCTCTANGG | 1 | 1 | 3 | 49 | NA |
| | (SEQ ID NO: 422) | | | | | |
| SP619.TIM3.g44 | GGGCACGAGGTTCCCTGGGGNGG | 1 | 1 | 3 | 53 | NA |
| | (SEQ ID NO: 423) | | | | | |
| SP619.TIM3.g38 | GGCTCCTTTGCCCCAGCAGANGG | 1 | 1 | 3 | 58 | NA |
| | (SEQ ID NO: 424) | | | | | |
| SP619.TIM3.g16 | ATTATTGGACATCCAGATACNGG | 1 | 1 | 3 | 106 | NA |
| | (SEQ ID NO: 425) | | | | | |
| SP619.TIM3.g28 | TTTCATCATTCATTATGCCTNGG | 1 | 1 | 4 | 23 | NA |
| | (SEQ ID NO: 426) | | | | | |
| SP619.TIM3.g4 | TTCTACACCCCAGCCGCCCCNGG | 1 | 1 | 4 | 29 | NA |
| | (SEQ ID NO: 427) | | | | | |
| SP619.TIM3.g34 | TCAGGGACACATCTCCTTTGNGG | 1 | 1 | 4 | 41 | NA |
| | (SEQ ID NO: 428) | | | | | |
| SP619.TIM3.g39 | GCTCCTTTGCCCCAGCAGACNGG | 1 | 1 | 4 | 42 | NA |
| | (SEQ ID NO: 429) | | | | | |
| SP619.TIM3.g1 | CTCAGAAGTGGAATACAGAGNGG | 1 | 1 | 5 | 35 | NA |
| | (SEQ ID NO: 430) | | | | | |
| SP619.TIM3.g46 | CGAGGTTCCCTGGGGCGGCTNGG | 1 | 2 | 2 | 18 | NA |
| | (SEQ ID NO: 431) | | | | | |
| SP619.TIM3.g37 | GCCACATTCAAACACAGGACNGG | 1 | 2 | 2 | 25 | NA |
| | (SEQ ID NO: 432) | | | | | |
| SP619.TIM3.g14 | GGTGCTCAGGACTGATGAAANGG | 1 | 2 | 3 | 28 | NA |
| | (SEQ ID NO: 433) | | | | | |

TABLE 26
Guide RNA (gRNA) Off Target Analysis for hTIM3 (Exon 3)

| Name | gRNA | long_0 | long_1 | long_2 | short_0 | SNP |
|---|---|---|---|---|---|---|
| SP620.TIM3.g1 | AGGTCACCCCTGCACCGACTNGG | 1 | 1 | 1 | 4 | rs1036199: 0.13 |
| | (SEQ ID NO: 434) | | | | | |
| SP620.TIM3.g11 | CTCTCTGCCGAGTCGGTGCANGG | 1 | 1 | 1 | 4 | rs1036199: 0.13 |
| | (SEQ ID NO: 435) | | | | | |
| SP620.TIM3.g10 | TCTCTCTGCCGAGTCGGTGCNGG | 1 | 1 | 1 | 6 | rs1036199: 0.13 |
| | (SEQ ID NO: 436) | | | | | |
| SP620.TIM3.g5 | CCAAGGATGCTTACCACCAGNGG | 1 | 1 | 1 | 8 | NA |
| | (SEQ ID NO: 437) | | | | | |
| SP620.TIM3.g12 | TCTCTGCCGAGTCGGTGCAGNGG | 1 | 1 | 1 | 9 | rs1036199: 0.13 |
| | (SEQ ID NO: 438) | | | | | |
| SP620.TIM3.g7 | CCCCTGGTGGTAAGCATCCTNGG | 1 | 1 | 1 | 10 | NA |
| | (SEQ ID NO: 439) | | | | | |
| SP620.TIM3.g4 | TCCAAGGATGCTTACCACCANGG | 1 | 1 | 1 | 16 | NA |
| | (SEQ ID NO: 440) | | | | | |
| SP620.TIM3.g8 | GGTGGTAAGCATCCTTGGAANGG | 1 | 1 | 1 | 20 | NA |
| | (SEQ ID NO: 441) | | | | | |
| SP620.TIM3.g9 | GTGAAGTCTCTCTGCCGAGTNGG | 1 | 1 | 2 | 6 | rs1036199: 0.13 |
| | (SEQ ID NO: 442) | | | | | |
| SP620.TIM3.g6 | ATGCTTACCACCAGGGGACANGG | 1 | 1 | 2 | 34 | NA |
| | (SEQ ID NO: 443) | | | | | |
| SP620.TIM3.g3 | TTCCAAGGATGCTTACCACCNGG | 1 | 1 | 2 | 36 | NA |
| | (SEQ ID NO: 444) | | | | | |
| SP620.TIM3.g13 | AGTCGGTGCAGGGGTGACCTNGG | 1 | 1 | 2 | 45 | NA |
| | (SEQ ID NO: 445) | | | | | |
| SP620.TIM3.g2 | ACTTCACTGCAGCCTTTCCANGG | 1 | 1 | 4 | 38 | NA |
| | (SEQ ID NO: 446) | | | | | |

The gRNA sequences in Table 25 and Table 26 were normalized (% Normalization to NHEJ) for gRNA activity via next generation sequencing (NGS). GFP was used as a control. Following sequencing analysis, the following gRNAs were recommended based on off-target profile: Exon 2: SP619.hTIM3.g12 (45.0%), SP619.hTIM3.g20 (60.9%), and SP619.hTIM3.g49 (45.4%). Exon 3: SP620.hTIM3.g5 (58.0%) and SP620.hTIM3.g7 (2.9%).

### Example 9—Method of Making and Testing BCMA-CAR-iNKT

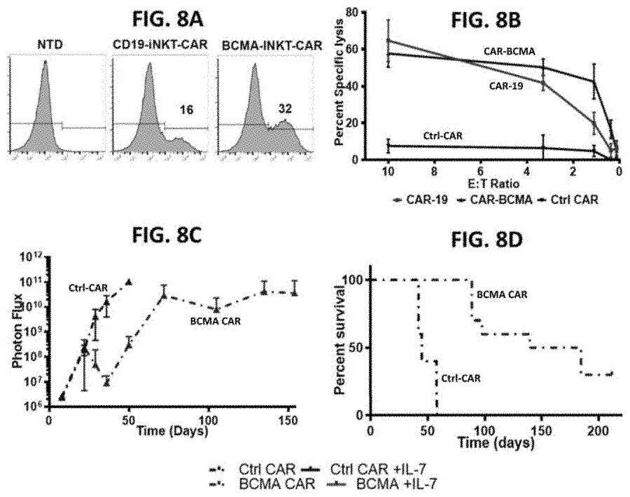

In a variation of the protocol in Example 1, CAR-iNKT recognizing a single antigen that can be made as in Step 1 and Step 2. Step 3 and Step 4 is omitted in this example. In Step 5, iNKT are transduced with a CAR that recognizes BCMA. CAR-iNKT cells produced by these methods are shown in FIG. 8A.

Efficacy of BCMA-CAR iNKT was tested in a 4 hr Cr release assay against BCMA+ target cells (MM1.$). FIG. 8B. In vivo efficacy was tested by engrafting 5×10⁵ MM.1S-CG i.v. into NSG mice and on day 28 mice were treated with 1×10e⁷BCMA-CAR-T or non-targeting CAR on day 28. Efficacy was assessed by measuring Tumor burden by BLI imaging FIG. 8C, and by monitoring survival, FIG. 8D.

### Example 10—CAR2-iNKT Cells Effectively Killing CD2+T-ALL and CTCL Cell Lines In Vitro

Step 1.

Peripheral blood mononuclear cells (PBMCs) were harvested from one or more healthy donors.

Step 2.

iNKT cells were isolated/purified from a donor's PBMCs, for example using magnetic selection with a labelled antibody-coated magnetic beads that bind to Valpha24 (e.g., Miltenyi Biotec). Other purification techniques are known in the art and could be used.

Step 3.

iNKT cells are thereafter activated. There are several ways to activate iNKT. The non iNKT fraction remaining after purification may be irradiated (e.g. at 40Gy) and dosed with α-GalCer (e.g., 200 ng/ml for 1 hr at, e.g., 37° C. to generate cells that have CD1d-α-GalCer, the ligand for the invariant receptor). iNKT cells are then incubated with irradiated α-GalCer pulsed negative cells (1:10). Alternatively, an anti-iNKT receptor antibody could be used to activate iNKT. In yet another alternative, purified CD-1d complexed with α-GalCer could be used to activate iNKT. In yet another alternative CD1d expressing cell line pulsed with α-GalCer could be used to stimulate iNKT

Step 4.

iNKT were counted and resuspended at 5×10⁶ in 100 ul electroporation buffer containing 20 ug CD2gRNA and 15 ug Cas9. iNKT were then transferred to a cuvette and electroporated using an electroporater known in the art. The cells were returned to pre-conditioned media.

Step 5.

iNKT were transduced with a CAR targeted to CD2.

Step 6.

iNKT were cultured to expand CAR-iNKT population. This can continue for several weeks. Regularly adding of α-GalCer loaded cells can keep the iNKT stimulated, but as with the initial stimulation, other options are available. The media typically contains high dose IL-2 (currently 200 units/ml in our protocol). IL-7, IL-15 or a combination of IL-2, IL-7 and IL-15 may also be used to expand iNKT in vitro. Analogues of these cytokines engineered to enhance potency or stability could also be used to enhance culture. Efficiency of CAR transduction (tested with CD34) and Target deletion (CD2) were assessed by flow cytometry.

Step 7.

Enrichment of CAR transduced iNKT were tested using trCD34 (marker of CAR expression) magnetic selection using the Miltenyi prodigy.

Step 8.

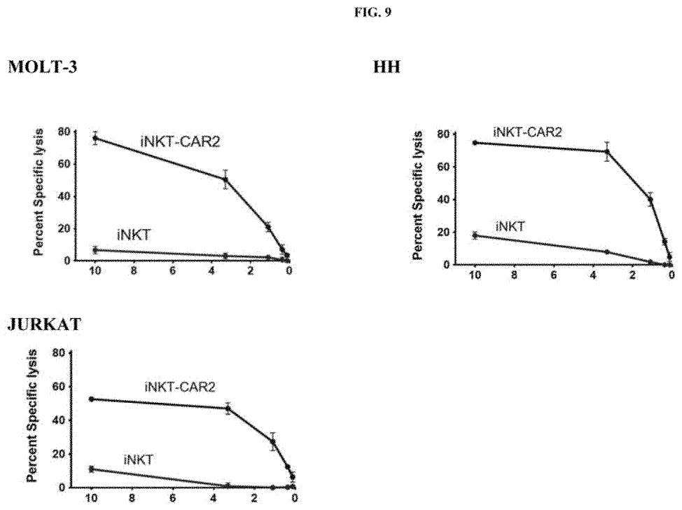

Efficacy of CAR-T killing was assessed by in vitro 4 hr Cr release assay against CD2+ target as shown in FIG. 9 where effectively killing CD2+T-ALL and CTCL cell lines in vitro was observed. A non-targeted cCAR-iNK was used as a control.

The methods disclosed above can be varied appropriately by those skilled in the art to make and confirm activity of other mono, dual, and tandem iNKT cells disclosed herein.

Although the present invention has been described with reference to specific details of certain embodiments thereof in the above examples, it will be understood that modification and variation are encompassed within the spirit and scope of the invention.

1.-2. (canceled)

3. An iNKT cell, which comprises at least one chimeric antigen receptor (CAR) targeting one or more antigens, and which is deficient in an antigen to which the CAR specifically binds.

4. The iNKT cell as recited in claim 3, wherein the chimeric antigen receptor specifically binds at least one antigen expressed on a malignant T cell.

5. The iNKT cell as recited in claim 4, wherein the antigen is selected from CD2, CD3ε, CD4, CD5, CD7, TRAC, and TCRβ.

6. The iNKT cell as recited in claim 3, wherein the chimeric antigen receptor specifically binds at least one antigen expressed on a malignant plasma cell.

7. The iNKT cell as recited in claim 6, wherein the antigen is selected from BCMA, CS1, CD38, and CD19.

8. The iNKT cell as recited in claim 3, wherein the chimeric antigen receptor expresses the extracellular portion of the APRIL protein, the ligand for BCMA and TACI, effectively co-targeting both BCMA and TACI.

9. (canceled)

10. The iNKT cell as recited in claim 1, wherein endogenous T cell receptor mediated signaling is negligible in the iNKT cell.

11. The iNKT cell as recited in claim 10, wherein the iNKT cells do not induce alloreactivity or graft-versus-host disease.

12. The iNKT cell as recited in claim 1, wherein the iNKT cells do not induce fratricide.

13.-24. (canceled)

24. A tandem iNKT-CAR cell, wherein the tandem iNKT-CAR cell comprises a linear tCAR construct.

25. The tandem iNKT-CAR cell as recited in claim 25, wherein the tandem iNKT cell comprises one CAR targeting a pair of (i.e., two) antigens.

26. The tandem iNKT-CAR cell as recited in claim 26, wherein the antigen pair is chosen from CD2×CD3ε, CD2×CD4, CD2×CD5, CD2×CD7, CD3εxCD4, CD3εxCD5, CD3εxCD7, CD4×CD5, CD4×CD7, CD5×CD7, TRAC×CD2, TRAC×CD3ε, TRAC×CD4, TRAC×CD5, TRAC×CD7, TCRβ×CD2, TCRβ×CD3ε, TCRβ×CD4, TCRβ×CD5, TCRβ×CD7, BCMA×CS1, BCMA×CD19, BCMA×CD38, CS1×CD19, CS1×CD38, CD19×CD38, APRIL×CS1, APRIL×BCMA, APRIL×CD19, and APRIL×CD38.

27. The tandem iNKT-CAR cell as recited in claim 25, wherein the linear tCAR construct comprises a first heavy (V_{H}) chain variable fragment and a first light (V_{L}) chain variable fragment, designated V_{H}1 and V_{L}1, joined by a (GGGGS)₂₋₆ linker to a second light (V_{L}) chain variable fragment and a first heavy (V_{H}) chain variable fragment, designated V_{L}2 and V_{H}2.

28. The tandem iNKT-CAR cell as recited in claim 25, wherein the linear tCAR construct comprises a first heavy (V_{H}) chain variable fragment and a first light (V_{L}) chain variable fragment, designated V_{H}2 and V_{L}2, joined by a (GGGGS)₂₋₆ linker to a second light (V_{L}) chain variable fragment and a first heavy (V_{H}) chain variable fragment, designated V_{H}1 and V_{L}1.

29. The tandem iNKT-CAR cell as recited in claim 25, wherein the linear tCAR construct comprises a first light (V_{L}) chain variable fragment and a first heavy (V_{H}) chain variable fragment, designated V_{L}1 and V_{H}1, joined by a (GGGGS)₂₋₆ linker to a second heavy (V_{H}) chain variable fragment and a first light (V_{L}) chain variable fragment, designated V_{H}2 and V_{L}2.

30. The tandem iNKT-CAR cell as recited in claim 25, wherein the linear tCAR construct comprises a first light (V_{L}) chain variable fragment and a first heavy (V_{H}) chain variable fragment, designated V_{L}2 and V_{H}2, joined by a (GGGGS)₂₋₆ linker to a second heavy (V_{H}) chain variable fragment and a first light (V_{L}) chain variable fragment, designated V_{H}1 and V_{L}1.

31. The tandem iNKT-CAR cell as recited in claim 25, wherein the linear tCAR construct comprises a structure chosen from 6-I to 6-XXXII.

32. A tandem iNKT-CAR cell, wherein the tandem iNKT-CAR cell comprises a hairpin tCAR construct.

33. The tandem iNKT-CAR cell as recited in claim 33, wherein the hairpin tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₂₋₆ linker to a first light (V_{L}) chain variable fragment derived from the second scFv, and a second light (V_{L}) chain variable fragment derived from the first scFv, designated V_{L}2 and V₁2.

34. The tandem iNKT-CAR cell as recited in claim 33, wherein the hairpin tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₂₋₆ linker to a first light (V_{L}) chain variable fragment derived from the first scFv, and a second light (V_{L}) chain variable fragment derived from the second scFv, designated V_{L}1 and V_{L}2.

35. The tandem iNKT-CAR cell as recited in claim 33, wherein the hairpin tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₂₋₆ linker to a first heavy (V_{H}) chain variable fragment derived from the first scFv, and a second heavy (V_{L}) chain variable fragment derived from the second scFv, designated V_{H}2 and V_{H}1.

36. The tandem iNKT-CAR cell as recited in claim 33, wherein the hairpin tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₂₋₆ linker to a first heavy (V_{H}) chain variable fragment derived from the first scFv, and a second light heavy (V_{H}) variable fragment derived from the second scFv, designated V_{H}1 and V_{H}2.

37. The tandem iNKT-CAR cell as recited in claim 33, wherein the hairpin tCAR construct comprises a structure chosen from 8-I to 8-XXXII.

38. The A tandem iNKT-CAR cell, wherein the tandem iNKT-CAR cell comprises a hairpin DSB tCAR construct with a (Cys=Cys) Double-Stranded Bond (DSB) in the linker.

39. The tandem iNKT-CAR cell as recited in claim 39, wherein the hairpin DSB tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ linker to a first light (V_{L}) chain variable fragment derived from the second scFv, and a second light (V_{L}) chain variable fragment derived from the first scFv, designated V_{L}2 and V₁2.

40. The tandem iNKT-CAR cell as recited in claim 39, wherein the hairpin DSB tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ linker to a first light (V_{L}) chain variable fragment derived from the first scFv, and a second light (V_{L}) chain variable fragment derived from the second scFv, designated V_{L}1 and V_{L}2.

41. The tandem iNKT-CAR cell as recited in claim 39, wherein the hairpin DSB tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₀₋₁-(GGGGC)₁₋(GGGGS)₁₋₂-(GGGGP)₁-(GGGGS)₂₋₃-(GGGGC)₁-(GGGGS)₀₋₁ linker to a first heavy (V_{H}) chain variable fragment derived from the first scFv, and a second heavy (V_{L}) chain variable fragment derived from the second scFv, designated V_{H}2 and V_{H}1.

42. The tandem iNKT-CAR cell as recited in claim 39, wherein the hairpin DSB tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₀₋₁-(GGGGC)₁₋(GGGGS)₁₋₂-(GGGGP)₁-(GGGGS)₂₋₃-(GGGGC)₁-(GGGGS)₀₋₁ linker to a first heavy (V_{H}) chain variable fragment derived from the first scFv, and a second light heavy (V_{H}) variable fragment derived from the second scFv, designated V_{H}1 and V_{H}2.

43. The tandem iNKT-CAR cell as recited in claim 39, wherein the hairpin DSB tCAR construct comprises a structure chosen from 10-I to 10-XXXII.

44.-46. (canceled)

47. The iNKT-CAR cell as recited in claim 25, wherein each of the V_{H} and V_{L} chains is different and displays at least 98% sequence identity to an amino acid sequence chosen from SEQ ID NO:12 to SEQ ID NO:31.

48. The iNKT-CAR cell as recited in claim 25, wherein each of the V_{H} and V_{L} chains is different and is a sequence chosen from SEQ ID NO:12 to SEQ ID NO:31.

49. The iNKT-CAR cell as recited in claim 25, comprising at least one costimulatory domain chosen from CD28 and 4-1 BB.

50. The iNKT-CAR cell as recited in claim 25, wherein the costimulatory domain is CD28.

51. The iNKT-CAR cell as recited in claim 25, comprising a CD3 signaling domain.

52. The iNKT-CAR cell as recited in claim 25, wherein the each of the V_{H} and V_{L} chains is derived from an scFv recognizing CD2 or an scFv recognizing CD3.

53. The iNKT-CAR cell as recited in claim 25, wherein the tCAR construct is chosen from Clone 5, Clone 6, Clone 7, Clone 8, Clone 13, Clone 14, Clone 15, and Clone 16.

54. The iNKT-CAR cell as recited in claim 25, wherein the tCAR construct displays at least 95% sequence identity to an amino acid sequence chosen from SEQ ID NO:41 to SEQ ID NO:46.

55.-59. (canceled)

60. A method of treatment of a hematologic malignancy in a patient comprising administering a tandem iNKT-CAR cell as recited in any of claims 25, 33, 39, to a patient in need thereof.

61. The method as recited in claim 60, wherein the hematologic malignancy is a T-cell malignancy.

62. The method as recited in claim 61, wherein the T cell malignancy is T-cell acute lymphoblastic leukemia (T-ALL).

63. The method as recited in claim 61, wherein the T cell malignancy is non-Hodgkins lymphoma.

64. The method as recited in claim 60, wherein the hematologic malignancy is multiple myeloma.

65. A method of making a gene-edited iNKT cell comprising the steps of:
a) activating isolated and purified iNKT cells;
b) deleting or suppressing expression of a cell surface protein in the iNKT cell; and
c) optionally, transducing the iNKT cell with a chimeric antigen receptor that recognizes one or more antigen or cell surface protein targets.

66. The method as recited in claim 65, which includes the step of transducing the iNKT cell with a chimeric antigen receptor that recognizes one or more antigen or cell surface protein targets.

67. The method as recited in claim 66, wherein the antigen that is the target of the CAR is deleted from the cell.

68. A method of making a population of genome-edited iNKT cells from multiple donors comprising the steps of:
a) activating isolated and purified iNKT cells from each donor;
b) deleting or suppressing expression of a cell surface protein in the iNKT cell;
c) optionally, transducing the iNKT cell with a chimeric antigen receptor that recognizes one or more antigen or cell surface protein targets;
d) expanding the population of genome-edited iNKT cells;
e) pooling the genome-edited iNKT cells.


SEQUENCE LISTING (451 sequences)

SEQ ID NO: 1 - protein, 21 aa, Artificial Sequence
  Description of Artificial Sequence Synthetic peptide
MALPVTALLLPLALLLHAARP

SEQ ID NO: 2 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 3 - protein, 27 aa, Artificial Sequence
  Description of Artificial Sequence Synthetic peptide
FWVLVVVGGVLACYSLLVTVAFIIFWV

SEQ ID NO: 4 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 5 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 6 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 7 - protein, 112 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 22 aa, Artificial Sequence
  Description of Artificial Sequence Synthetic peptide
GSGATNFSLLKQAGDVEENPGP

SEQ ID NO: 9 - protein, 20 aa, Artificial Sequence
  Description of Artificial Sequence Synthetic peptide
GGGGSGGGGSGGGGSGGGGS

SEQ ID NO: 10 - protein, 308 aa, Artificial Sequence
  Description of Artificial Sequence Synthetic polypeptide
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 11 - protein, 710 aa, Artificial Sequence
  Description of Artificial Sequence Synthetic polypeptide
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 12 - protein, 119 aa, Artificial Sequence
  Description of Artificial Sequence Synthetic polypeptide
EVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSS

SEQ ID NO: 13 - protein, 109 aa, Artificial Sequence
  Description of Artificial Sequence Synthetic polypeptide
DIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKR

SEQ ID NO: 14 - protein, 121 aa, Artificial Sequence
  Description of Artificial Sequence Synthetic polypeptide
EVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSS

SEQ ID NO: 15 - protein, 113 aa, Artificial Sequence
  Description of Artificial Sequence Synthetic polypeptide
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIER

SEQ ID NO: 16 - protein, 121 aa, Artificial Sequence
  Description of Artificial Sequence Synthetic polypeptide
GSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 17 - protein, 107 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 18 - protein, 122 aa, Artificial Sequence
  Description of Artificial Sequence Synthetic polypeptide
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 19 - protein, 107 aa, Artificial Sequence
  Description of Artificial Sequence Synthetic polypeptide
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 20 - protein, 116 aa, Artificial Sequence
  Description of Artificial Sequence Synthetic polypeptide
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVS

SEQ ID NO: 21 - protein, 108 aa, Artificial Sequence
  Description of Artificial Sequence Synthetic polypeptide
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 22 - protein, 119 aa, Artificial Sequence
  Description of Artificial Sequence Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 23 - protein, 112 aa, Artificial Sequence
  Description of Artificial Sequence Synthetic polypeptide
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 24 - protein, 122 aa, Artificial Sequence
  Description of Artificial Sequence Synthetic polypeptide
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 25 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 26 - protein, 118 aa, Artificial Sequence
  Description of Artificial Sequence Synthetic polypeptide
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 27 - protein, 112 aa, Artificial Sequence
  Description of Artificial Sequence Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 28 - protein, 120 aa, Artificial Sequence
  Description of Artificial Sequence Synthetic polypeptide
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 29 - protein, 107 aa, Artificial Sequence
  Description of Artificial Sequence Synthetic polypeptide
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 30 - protein, 120 aa, Artificial Sequence
  Description of Artificial Sequence Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSG

SEQ ID NO: 31 - protein, 115 aa, Artificial Sequence
  Description of Artificial Sequence Synthetic polypeptide
GSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 32 - protein, 832 aa, Artificial Sequence
  Description of Artificial Sequence Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 33 - protein, 1234 aa, Artificial Sequence
  Description of Artificial Sequence Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 34 - protein, 831 aa, Artificial Sequence
  Description of Artificial Sequence Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 35 - protein, 1233 aa, Artificial Sequence
  Description of Artificial Sequence Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 36 - protein, 1248 aa, Artificial Sequence
  Description of Artificial Sequence Synthetic polypeptide
MALPVTALLLPLALLLHAARPGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRGGGGSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGFKRDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 37 - protein, 841 aa, Artificial Sequence
  Description of Artificial Sequence Synthetic polypeptide
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 38 - protein, 842 aa, Artificial Sequence
  Description of Artificial Sequence Synthetic polypeptide
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 39 - protein, 835 aa, Artificial Sequence
  Description of Artificial Sequence Synthetic polypeptide
MALPVTALLLPLALLLHAARPGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 40 - protein, 835 aa, Artificial Sequence; identical to SEQ ID NO: 39
  Description of Artificial Sequence Synthetic polypeptide

SEQ ID NO: 41 - protein, 1133 aa, Artificial Sequence
  Description of Artificial Sequence Synthetic polypeptide
MALPVTALLLPLALLLHAARPQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 42 - protein, 1134 aa, Artificial Sequence
  Description of Artificial Sequence Synthetic polypeptide
MALPVTALLLPLALLLHAARPDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSEVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 43 - protein, 1134 aa, Artificial Sequence
  Description of Artificial Sequence Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSGGGGSGGGGSGGGGSGGGGSGGGGSGGGGSGGGGSGGGGSGGGGSEVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 44 - protein, 1133 aa, Artificial Sequence
  Description of Artificial Sequence Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSGGGGSGGGGSGGGGSGGGGSGGGGSGGGGSGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 45 - protein, 1129 aa, Artificial Sequence
  Description of Artificial Sequence Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 46 - protein, 1129 aa, Artificial Sequence
  Description of Artificial Sequence Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSGGGGSGGGGSGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 47 - RNA, 100 nt, Artificial Sequence
  Description of Artificial Sequence Synthetic polynucleotide
aucacggaggucaaugucuaguuuuagagcuagaaauagcaaguuaaaauaaggcuaguccguuaucaacuugaaaaaguggcaccgagucggugcuuuu

SEQ ID NO: 48 - RNA, 100 nt, Artificial Sequence
  Description of Artificial Sequence Synthetic polynucleotide
guagacauugaccuccgugaguuuuagagcuagaaauagcaaguuaaaauaaggcuaguccguuaucaacuugaaaaaguggcaccgagucggugcuuuu

SEQ ID NO: 49 - RNA, 100 nt, Artificial Sequence; identical to SEQ ID NO: 47
  Description of Artificial Sequence Synthetic polynucleotide

SEQ ID NO: 50 - RNA, 100 nt, Artificial Sequence
  Description of Artificial Sequence Synthetic polynucleotide
gagaaucaaaaucggugaauguuuuagagcuagaaauagcaaguuaaaauaaggcuaguccguuaucaacuugaaaaaguggcaccgagucggugcuuuu

SEQ ID NO: 51 - RNA, 100 nt, Artificial Sequence
  Description of Artificial Sequence Synthetic polynucleotide
gaccaaucugacaugcugcaguuuuagagcuagaaauagcaaguuaaaauaaggcuaguccguuaucaacuugaaaaaguggcaccgagucggugcuuuu

SEQ ID NO: 52 - RNA, 100 nt, Artificial Sequence
  Description of Artificial Sequence Synthetic polynucleotide
acagcugacaggcucgacacguuuuagagcuagaaauagcaaguuaaaauaaggcuaguccguuaucaacuugaaaaaguggcaccgagucggugcuuuu

SEQ ID NO: 53 - RNA, 100 nt, Artificial Sequence; identical to SEQ ID NO: 50
  Description of Artificial Sequence Synthetic polynucleotide

SEQ ID NO: 54 - RNA, 100 nt, Artificial Sequence
  Description of Artificial Sequence Synthetic polynucleotide
agggcaugucaauauuacugguuuuagagcuagaaauagcaaguuaaaauaaggcuaguccguuaucaacuugaaaaaguggcaccgagucggugcuuuu

SEQ ID NO: 55 - RNA, 100 nt, Artificial Sequence
  Description of Artificial Sequence Synthetic polynucleotide
cguuccaacucgaagugccaguuuuagagcuagaaauagcaaguuaaaauaaggcuaguccguuaucaacuugaaaaaguggcaccgagucggugcuuuu

SEQ ID NO: 56 - RNA, 100 nt, Artificial Sequence; identical to SEQ ID NO: 55
  Description of Artificial Sequence Synthetic polynucleotide

SEQ ID NO: 57 - DNA, 23 bp, Artificial Sequence
  Description of Artificial Sequence Synthetic polynucleotide
  misc_feature (21)..(21): n is a, c, g, or t
caaagagattacgaatgcctngg

SEQ ID NO: 58 - DNA, 23 bp, Artificial Sequence
  Description of Artificial Sequence Synthetic polynucleotide
  misc_feature (21)..(21): n is a, c, g, or t
caaggcattcgtaatctcttngg

SEQ ID NO: 59 - DNA, 23 bp, Artificial Sequence
  Description of Artificial Sequence Synthetic polynucleotide
  misc_feature (21)..(21): n is a, c, g, or t
cttgtagatatcctgatcatngg

SEQ ID NO: 60 - DNA, 23 bp, Artificial Sequence
  Description of Artificial Sequence Synthetic polynucleotide
  misc_feature (21)..(21): n is a, c, g, or t
cttgggtcaggacatcaactngg

SEQ ID NO: 61 - DNA, 23 bp, Artificial Sequence
  Description of Artificial Sequence Synthetic polynucleotide
  misc_feature (21)..(21): n is a, c, g, or t
cgatgatcaggatatctacangg

SEQ ID NO: 62 - DNA, 23 bp, Artificial Sequence
  Description of Artificial Sequence Synthetic polynucleotide
  misc_feature (21)..(21): n is a, c, g, or t
ttacgaatgccttggaaaccngg

SEQ ID NO: 63 - DNA, 23 bp, Artificial Sequence
  Description of Artificial Sequence Synthetic polynucleotide
  misc_feature (21)..(21): n is a, c, g, or t
tacgaatgccttggaaacctngg

SEQ ID NO: 64 - DNA, 23 bp, Artificial Sequence
  Description of Artificial Sequence Synthetic polynucleotide
  misc_feature (21)..(21): n is a, c, g, or t
acgaatgccttggaaacctgngg

SEQ ID NO: 65 - DNA, 23 bp, Artificial Sequence
  Description of Artificial Sequence Synthetic polynucleotide
  misc_feature (21)..(21): n is a, c, g, or t
tgatattgacgatataaaatngg

SEQ ID NO: 66 - DNA, 23 bp, Artificial Sequence
  Description of Artificial Sequence Synthetic polynucleotide
  misc_feature (21)..(21): n is a, c, g, or t
atgatattgacgatataaaangg

SEQ ID NO: 67 - DNA, 23 bp, Artificial Sequence
  Description of Artificial Sequence Synthetic polynucleotide
  misc_feature (21)..(21): n is a, c, g, or t
gcatctgaagaccgatgatcngg

SEQ ID NO: 68 - DNA, 23 bp, Artificial Sequence
  Description of Artificial Sequence Synthetic polynucleotide
  misc_feature (21)..(21): n is a, c, g, or t
aacctggggtgccttgggtcngg

SEQ ID NO: 69 - DNA, 23 bp, Artificial Sequence
  Description of Artificial Sequence Synthetic polynucleotide
  misc_feature (21)..(21): n is a, c, g, or t
ttggaaacctggggtgccttngg

SEQ ID NO: 70 - DNA, 23 bp, Artificial Sequence
  Description of Artificial Sequence Synthetic polynucleotide
  misc_feature (21)..(21): n is a, c, g, or t
gtatcaatatatgatacaaangg

SEQ ID NO: 71 - DNA, 23 bp, Artificial Sequence
  Description of Artificial Sequence Synthetic polynucleotide
  misc_feature (21)..(21): n is a, c, g, or t
caaggcaccccaggtttccangg

SEQ ID NO: 72 - DNA, 23 bp, Artificial Sequence
  Description of Artificial Sequence Synthetic polynucleotide
  misc_feature (21)..(21): n is a, c, g, or t
cttggaaacctggggtgcctngg

SEQ ID NO: 73 - DNA, 23 bp, Artificial Sequence
  Description of Artificial Sequence Synthetic polynucleotide
  misc_feature (21)..(21): n is a, c, g, or t
tcatcactcatttgaaaactngg

SEQ ID NO: 74 - DNA, 23 bp, Artificial Sequence
  Description of Artificial Sequence Synthetic polynucleotide
  misc_feature (21)..(21): n is a, c, g, or t
caagttgatgtcctgacccangg

SEQ ID NO: 75 - DNA, 23 bp, Artificial Sequence
  Description of Artificial Sequence Synthetic polynucleotide
  misc_feature (21)..(21): n is a, c, g, or t
gtcctgacccaaggcaccccngg

SEQ ID NO: 76 - DNA, 23 bp, Artificial Sequence
  Description of Artificial Sequence Synthetic polynucleotide
  misc_feature (21)..(21): n is a, c, g, or t
atatttgatttgaagattcangg

SEQ ID NO: 77 - DNA, 23 bp, Artificial Sequence
  Description of Artificial Sequence Synthetic polynucleotide
  misc_feature (21)..(21): n is a, c, g, or t
tacaaaaggaaaaaatgtgtngg

SEQ ID NO: 78 - DNA, 23 bp, Artificial Sequence
  Description of Artificial Sequence Synthetic polynucleotide
  misc_feature (21)..(21): n is a, c, g, or t
acatataagctatttaaaaangg

SEQ ID NO: 79 - DNA, 23 bp, Artificial Sequence
  Description of Artificial Sequence Synthetic polynucleotide
  misc_feature (21)..(21): n is a, c, g, or t
aaaagagaaagagactttcangg

SEQ ID NO: 80 - DNA, 23 bp, Artificial Sequence
  Description of Artificial Sequence Synthetic polynucleotide
  misc_feature (21)..(21): n is a, c, g, or t
cttgatacaggtttaattcgngg

SEQ ID NO: 81 - DNA, 23 bp, Artificial Sequence
  Description of Artificial Sequence Synthetic polynucleotide
  misc_feature (21)..(21): n is a, c, g, or t
acagctgacaggctcgacacngg

SEQ ID NO: 82 - DNA, 23 bp, Artificial Sequence
  Description of Artificial Sequence Synthetic polynucleotide
  misc_feature (21)..(21): n is a, c, g, or t
gatgtttcccatcttgatacngg

SEQ ID NO: 83 - DNA, 23 bp, Artificial Sequence
  Description of Artificial Sequence Synthetic polynucleotide
  misc_feature (21)..(21): n is a, c, g, or t
gtcgagcctgtcagctgtccngg

SEQ ID NO: 84 - DNA, 23 bp, Artificial Sequence
  Description of Artificial Sequence Synthetic polynucleotide
  misc_feature (21)..(21): n is a, c, g, or t
caaaattcaagtgcacagcangg

SEQ ID NO: 85 - DNA, 23 bp, Artificial Sequence
  Description of Artificial Sequence Synthetic polynucleotide
  misc_feature (21)..(21): n is a, c, g, or t
gaattttgcactcaggctggngg

SEQ ID NO: 86 - DNA, 23 bp, Artificial Sequence
  Description of Artificial Sequence Synthetic polynucleotide
  misc_feature (21)..(21): n is a, c, g, or t
gaattaaacctgtatcaagangg

SEQ ID NO: 87 - DNA, 23 bp, Artificial Sequence
  Description of Artificial Sequence Synthetic polynucleotide
  misc_feature (21)..(21): n is a, c, g, or t
aattaaacctgtatcaagatngg

SEQ ID NO: 88 - DNA, 23 bp, Artificial Sequence
  Description of Artificial Sequence Synthetic polynucleotide
  misc_feature (21)..(21): n is a, c, g, or t
agttccattcattacctcacngg

SEQ ID NO: 89 - DNA, 23 bp, Artificial Sequence
  Description of Artificial Sequence Synthetic polynucleotide
  misc_feature (21)..(21): n is a, c, g, or t
agagggtcatcacacacaagngg

SEQ ID NO: 90 - DNA, 23 bp, Artificial Sequence
  Description of Artificial Sequence Synthetic polynucleotide
  misc_feature (21)..(21): n is a, c, g, or t
atacaagtccaggagatcttngg

SEQ ID NO: 91 - DNA, 23 bp, Artificial Sequence
  Description of Artificial Sequence Synthetic polynucleotide
  misc_feature (21)..(21): n is a, c, g, or t
tcttgatacaggtttaattcngg

SEQ ID NO: 92 - DNA, 23 bp, Artificial Sequence
  Description of Artificial Sequence Synthetic polynucleotide
  misc_feature (21)..(21): n is a, c, g, or t
ctgacctgtgaggtaatgaangg

SEQ ID NO: 93 - DNA, 23 bp, Artificial Sequence
  Description of Artificial Sequence Synthetic polynucleotide
  misc_feature (21)..(21): n is a, c, g, or t
acatctaaaactttctcagangg

SEQ ID NO: 94 - DNA, 23 bp, Artificial Sequence
  Description of Artificial Sequence Synthetic polynucleotide
  misc_feature (21)..(21): n is a, c, g, or t
gcaaaattcaagtgcacagcngg

SEQ ID NO: 95 - DNA, 23 bp, Artificial Sequence
  Description of Artificial Sequence Synthetic polynucleotide
  misc_feature (21)..(21): n is a, c, g, or t
ggttgtgttgatacaagtccngg

SEQ ID NO: 96 - DNA, 23 bp, Artificial Sequence
  Description of Artificial Sequence Synthetic polynucleotide
  misc_feature (21)..(21): n is a, c, g, or t
atcttgatacaggtttaattngg

SEQ ID NO: 97 - DNA, 23 bp, Artificial Sequence
  Description of Artificial Sequence Synthetic polynucleotide
  misc_feature (21)..(21): n is a, c, g, or t
attcattacctcacaggtcangg

SEQ ID NO: 98 - DNA, 23 bp, Artificial Sequence
  Description of Artificial Sequence Synthetic polynucleotide
  misc_feature (21)..(21): n is a, c, g, or t
aacatctaaaactttctcagngg

SEQ ID NO: 99 - DNA, 23 bp, Artificial Sequence
  Description of Artificial Sequence Synthetic polynucleotide
  misc_feature (21)..(21): n is a, c, g, or t
agcagggaacaaagtcagcangg

SEQ ID NO: 100 - DNA, 23 bp, Artificial Sequence
  Description of Artificial Sequence Synthetic polynucleotide
  misc_feature (21)..(21): n is a, c, g, or t
caacacaaccctgacctgtgngg

SEQ ID NO: 101 - DNA, 23 bp, Artificial Sequence
  Description of Artificial Sequence Synthetic polynucleotide
  misc_feature (21)..(21): n is a, c, g, or t
cttgaattttgcactcaggcngg

SEQ ID NO: 102 - DNA, 23 bp, Artificial Sequence
  Description of Artificial Sequence Synthetic polynucleotide
  misc_feature (21)..(21): n is a, c, g, or t
cattcattacctcacaggtcngg

SEQ ID NO: 103 - DNA, 23 bp, Artificial Sequence
  Description of Artificial Sequence Synthetic polynucleotide
  misc_feature (21)..(21): n is a, c, g, or t
tgcacttgaattttgcactcngg

SEQ ID NO: 104 - DNA, 23 bp, Artificial Sequence
  Description of Artificial Sequence Synthetic polynucleotide
  misc_feature (21)..(21): n is a, c, g, or t
tctcaaaaccaaagatctccngg

SEQ ID NO: 105 - DNA, 23 bp, Artificial Sequence
  Description of Artificial Sequence Synthetic polynucleotide
  misc_feature (21)..(21): n is a, c, g, or t
ttgacatgccctcagtatccngg

SEQ ID NO: 106 - DNA, 23 bp, Artificial Sequence
  Description of Artificial Sequence Synthetic polynucleotide
  misc_feature (21)..(21): n is a, c, g, or t
ctggattacctcttgccctcngg

SEQ ID NO: 107 - DNA, 23 bp, Artificial Sequence
  Description of Artificial Sequence Synthetic polynucleotide
  misc_feature (21)..(21): n is a, c, g, or t
gagatggagactttatatgcngg

SEQ ID NO: 108 - DNA, 23 bp, Artificial Sequence
  Description of Artificial Sequence Synthetic polynucleotide
  misc_feature (21)..(21): n is a, c, g, or t
agatggagactttatatgctngg

SEQ ID NO: 109 - DNA, 23 bp, Artificial Sequence
  Description of Artificial Sequence Synthetic polynucleotide
  misc_feature (21)..(21): n is a, c, g, or t
agggcatgtcaatattactgngg

SEQ ID NO: 110 - DNA, 23 bp, Artificial Sequence
  Description of Artificial Sequence Synthetic polynucleotide
  misc_feature (21)..(21): n is a, c, g, or t
gatggagactttatatgctgngg

SEQ ID NO: 111 - DNA, 23 bp, Artificial Sequence
  Description of Artificial Sequence Synthetic polynucleotide
  misc_feature (21)..(21): n is a, c, g, or t
tattatgtctgctaccccagngg

SEQ ID NO: 112 - DNA, 23 bp, Artificial Sequence
  Description of Artificial Sequence Synthetic polynucleotide
  misc_feature (21)..(21): n is a, c, g, or t
tgccatagtatttcagatccngg

SEQ ID NO: 113 - DNA, 23 bp, Artificial Sequence
  Description of Artificial Sequence Synthetic polynucleotide
  misc_feature (21)..(21): n is a, c, g, or t
agataaaagttcgcatcttcngg

SEQ ID NO: 114 - DNA, 23 bp, Artificial Sequence
  Description of Artificial Sequence Synthetic polynucleotide
  misc_feature (21)..(21): n is a, c, g, or t
ctgaaaattccttcagtgacngg

SEQ ID NO: 115 - DNA, 23 bp, Artificial Sequence
  Description of Artificial Sequence Synthetic polynucleotide
  misc_feature (21)..(21): n is a, c, g, or t
ctgagggcaagaggtaatccngg

SEQ ID NO: 116 - DNA, 23 bp, Artificial Sequence
  Description of Artificial Sequence Synthetic polynucleotide
  misc_feature (21)..(21): n is a, c, g, or t
tttcagatccaggatactgangg

SEQ ID NO: 117 - DNA, 23 bp, Artificial Sequence
  Description of Artificial Sequence Synthetic polynucleotide
  misc_feature (21)..(21): n is a, c, g, or t
tatctctacctgagggcaagngg

SEQ ID NO: 118 - DNA, 23 bp, Artificial Sequence
  Description of Artificial Sequence Synthetic polynucleotide
  misc_feature (21)..(21): n is a, c, g, or t
tgaggatcacctgtcactgangg

SEQ ID NO: 119 - DNA, 23 bp, Artificial Sequence
  Description of Artificial Sequence Synthetic polynucleotide
  misc_feature (21)..(21): n is a, c, g, or t
aatcatctgctacggacaacngg

SEQ ID NO: 120 - DNA, 23 bp, Artificial Sequence
  Description of Artificial Sequence Synthetic polynucleotide
  misc_feature (21)..(21): n is a, c, g, or t
gcagacttttgacgcttgacngg

SEQ ID NO: 121 - DNA, 23 bp, Artificial Sequence
  Description of Artificial Sequence Synthetic polynucleotide
  misc_feature (21)..(21): n is a, c, g, or t
ccgttccaactcgaagtgccngg

SEQ ID NO: 122 - DNA, 23 bp, Artificial Sequence
  Description of Artificial Sequence Synthetic polynucleotide
  misc_feature (21)..(21): n is a, c, g, or t
cgttccaactcgaagtgccangg

SEQ ID NO: 123 - DNA, 23 bp, Artificial Sequence
  Description of Artificial Sequence Synthetic polynucleotide
  misc_feature (21)..(21): n is a, c, g, or t
ctggcacttcgagttggaacngg

SEQ ID NO: 124 - DNA, 23 bp, Artificial Sequence
  Description of Artificial Sequence Synthetic polynucleotide
  misc_feature (21)..(21): n is a, c, g, or t
gtctgccagcggctgaactgngg

SEQ ID NO: 125 - DNA, 23 bp, Artificial Sequence
  Description of Artificial Sequence Synthetic polynucleotide
  misc_feature (21)..(21): n is a, c, g, or t
atcatctgctacggacaactngg

SEQ ID NO: 126 - DNA, 23 bp, Artificial Sequence
  Description of Artificial Sequence Synthetic polynucleotide
  misc_feature (21)..(21): n is a, c, g, or t
agacttttgacgcttgactgngg

SEQ ID NO: 127 - DNA, 23 bp, Artificial Sequence
  Description of Artificial Sequence Synthetic polynucleotide
  misc_feature (21)..(21): n is a, c, g, or t
cagacttttgacgcttgactngg

SEQ ID NO: 128 - DNA, 23 bp, Artificial Sequence
  Description of Artificial Sequence Synthetic polynucleotide
  misc_feature (21)..(21): n is a, c, g, or t
cctggcacttcgagttggaangg

SEQ ID NO: 129 - DNA, 23 bp, Artificial Sequence
  Description of Artificial Sequence Synthetic polynucleotide
  misc_feature (21)..(21): n is a, c, g, or t
gcaccccacagttcagccgcngg

SEQ ID NO: 130 - DNA, 23 bp, Artificial Sequence
  Description of Artificial Sequence Synthetic polynucleotide
  misc_feature (21)..(21): n is a, c, g, or t
ccttgaggtagacctccagcngg

SEQ ID NO: 131 - DNA, 23 bp, Artificial Sequence
  Description of Artificial Sequence Synthetic polynucleotide
  misc_feature (21)..(21): n is a, c, g, or t
aggtctacctcaaggacggangg

SEQ ID NO: 132 - DNA, 23 bp, Artificial Sequence
  Description of Artificial Sequence Synthetic polynucleotide
  misc_feature (21)..(21): n is a, c, g, or t
tggaacgggtgagccttgccngg

SEQ ID NO: 133 - DNA, 23 bp, Artificial Sequence
  Description of Artificial Sequence Synthetic polynucleotide
  misc_feature (21)..(21): n is a, c, g, or t
tgtggggtgcccttaagcctngg

SEQ ID NO: 134 - DNA, 23 bp, Artificial Sequence
  Description of Artificial Sequence Synthetic polynucleotide
  misc_feature (21)..(21): n is a, c, g, or t
aagcgtcaaaagtctgccagngg

SEQ ID NO: 135 - DNA, 23 bp, Artificial Sequence
  Description of Artificial Sequence Synthetic polynucleotide
  misc_feature (21)..(21): n is a, c, g, or t
tagcagatgattgagctctgngg

SEQ ID NO: 136 - DNA, 23 bp, Artificial Sequence
  Description of Artificial Sequence Synthetic polynucleotide
  misc_feature (21)..(21): n is a, c, g, or t
gattgagctctgaggtgtgtngg

SEQ ID NO: 137 - DNA, 23 bp, Artificial Sequence
  Description of Artificial Sequence Synthetic polynucleotide
  misc_feature (21)..(21): n is a, c, g, or t
ggggccggagctccaagcagngg

SEQ ID NO: 138 - DNA, 23 bp, Artificial Sequence
  Description of Artificial Sequence Synthetic polynucleotide
  misc_feature (21)..(21): n is a, c, g, or t
ggtgtgtaggtgacaaggaangg

SEQ ID NO: 139 - DNA, 23 bp, Artificial Sequence
  Description of Artificial Sequence Synthetic polynucleotide
  misc_feature (21)..(21): n is a, c, g, or t
ccggagctccaagcagtgggngg

SEQ ID NO: 140 - DNA, 23 bp, Artificial Sequence
  Description of Artificial Sequence Synthetic polynucleotide
  misc_feature (21)..(21): n is a, c, g, or t
ggtagacctccagctggcccngg

SEQ ID NO: 141 - DNA, 23 bp, Artificial Sequence
  Description of Artificial Sequence Synthetic polynucleotide
  misc_feature (21)..(21): n is a, c, g, or t
ctcgaagtgccagggccagcngg

SEQ ID NO: 142 - DNA, 23 bp, Artificial Sequence
  Description of Artificial Sequence Synthetic polynucleotide
  misc_feature (21)..(21): n is a, c, g, or t
ctggccctggcacttcgagtngg

SEQ ID NO: 143 - DNA, 23 bp, Artificial Sequence
  Description of Artificial Sequence Synthetic polynucleotide
  misc_feature (21)..(21): n is a, c, g, or t
tctgccagcggctgaactgtngg

SEQ ID NO: 144 - DNA, 23 bp, Artificial Sequence
  Description of Artificial Sequence Synthetic polynucleotide
  misc_feature (21)..(21): n is a, c, g, or t
ccatgtgccatccgtccttgngg

SEQ ID NO: 145 - DNA, 23 bp, Artificial Sequence
  Description of Artificial Sequence Synthetic polynucleotide
  misc_feature (21)..(21): n is a, c, g, or t
ccagctggaggtctacctcangg

SEQ ID NO: 146 - DNA, 23 bp, Artificial Sequence
  Description of Artificial Sequence Synthetic polynucleotide
  misc_feature (21)..(21): n is a, c, g, or t
tctgaggtgtgtaggtgacangg

SEQ ID NO: 147 - DNA, 23 bp, Artificial Sequence
  Description of Artificial Sequence Synthetic polynucleotide
  misc_feature (21)..(21): n is a, c, g, or t
aggaaggggccaaggcttaangg

SEQ ID NO: 148 - DNA, 23 bp, Artificial Sequence
  Description of Artificial Sequence Synthetic polynucleotide
  misc_feature (21)..(21): n is a, c, g, or t
cagagctcaatcatctgctangg

SEQ ID NO: 149 - DNA, 23 bp, Artificial Sequence
  Description of Artificial Sequence Synthetic polynucleotide
  misc_feature (21)..(21): n is a, c, g, or t
gggccggagctccaagcagtngg

SEQ ID NO: 150 - DNA, 23 bp, Artificial Sequence
  Description of Artificial Sequence Synthetic polynucleotide
  misc_feature (21)..(21): n is a, c, g, or t
cctcccactgcttggagctcngg

SEQ ID NO: 151 - DNA, 23 bp, Artificial Sequence
  Description of Artificial Sequence Synthetic polynucleotide
  misc_feature (21)..(21): n is a, c, g, or t
tggagctccggccccagctcngg

SEQ ID NO: 152 - DNA, 23 bp, Artificial Sequence
  Description of Artificial Sequence Synthetic polynucleotide
  misc_feature (21)..(21): n is a, c, g, or t
gtgtgtaggtgacaaggaagngg

SEQ ID NO: 153 - DNA, 23 bp, Artificial Sequence
  Description of Artificial Sequence Synthetic polynucleotide
  misc_feature (21)..(21): n is a, c, g, or t
atggtttgcagccagagctgngg

SEQ ID NO: 154 - DNA, 23 bp, Artificial Sequence
  Description of Artificial Sequence Synthetic polynucleotide
  misc_feature (21)..(21): n is a, c, g, or t
ctggaggtctacctcaaggangg

SEQ ID NO: 155 - DNA, 23 bp, Artificial Sequence
  Description of Artificial Sequence Synthetic polynucleotide
  misc_feature (21)..(21): n is a, c, g, or t
ctgccagcggctgaactgtgngg

SEQ ID NO: 156 - DNA, 23 bp, Artificial Sequence
  Description of Artificial Sequence Synthetic polynucleotide
  misc_feature (21)..(21): n is a, c, g, or t
aatgacatgtgtcactctctngg

SEQ ID NO: 157 - DNA, 23 bp, Artificial Sequence
  Description of Artificial Sequence Synthetic polynucleotide
  misc_feature (21)..(21): n is a, c, g, or t
acatggtttgcagccagagcngg

SEQ ID NO: 158 - DNA, 23 bp, Artificial Sequence
  Description of Artificial Sequence Synthetic polynucleotide
  misc_feature (21)..(21): n is a, c, g, or t
catggtttgcagccagagctngg

SEQ ID NO: 159 - DNA, 23 bp, Artificial Sequence
  Description of Artificial Sequence Synthetic polynucleotide
  misc_feature (21)..(21): n is a, c, g, or t
gacacatgtcatttctgctgngg

SEQ ID NO: 160 - DNA, 23 bp, Artificial Sequence
  Description of Artificial Sequence Synthetic polynucleotide
  misc_feature (21)..(21): n is a, c, g, or t
actggggtcctcccactgctngg

SEQ ID NO: 161 - DNA, 23 bp, Artificial Sequence
  Description of Artificial Sequence Synthetic polynucleotide
  misc_feature (21)..(21): n is a, c, g, or t
cctcaaggacggatggcacangg

SEQ ID NO: 162 - DNA, 23 bp, Artificial Sequence
  Description of Artificial Sequence Synthetic polynucleotide
  misc_feature (21)..(21): n is a, c, g, or t
aggtgtgtaggtgacaaggangg

SEQ ID NO: 163 - DNA, 23 bp, Artificial Sequence
  Description of Artificial Sequence Synthetic polynucleotide
  misc_feature (21)..(21): n is a, c, g, or t
aaggaaggggccaaggcttangg

SEQ ID NO: 164 - DNA, 23 bp, Artificial Sequence
  Description of Artificial Sequence Synthetic polynucleotide
  misc_feature (21)..(21): n is a, c, g, or t
gaagtgccagggccagctggngg

SEQ ID NO: 165 - DNA, 23 bp, Artificial Sequence
  Description of Artificial Sequence Synthetic polynucleotide
  misc_feature (21)..(21): n is a, c, g, or t
tttgcagccagagctggggcngg

SEQ ID NO: 166 - DNA, 23 bp, Artificial Sequence
  Description of Artificial Sequence Synthetic polynucleotide
  misc_feature (21)..(21): n is a, c, g, or t
aaatgacatgtgtcactctcngg

SEQ ID NO: 167 - DNA, 23 bp, Artificial Sequence
  Description of Artificial Sequence Synthetic polynucleotide
  misc_feature (21)..(21): n is a, c, g, or t
aggtgacaaggaaggggccangg

SEQ ID NO: 168 - DNA, 23 bp, Artificial Sequence
  Description of Artificial Sequence Synthetic polynucleotide
  misc_feature (21)..(21): n is a, c, g, or t
atttctgctgtggctgcagtngg

SEQ ID NO: 169 - DNA, 23 bp, Artificial Sequence
  Description of Artificial Sequence Synthetic polynucleotide
  misc_feature (21)..(21): n is a, c, g, or t
gctgtggctgcagttggagangg

SEQ ID NO: 170 - DNA, 23 bp, Artificial Sequence
  Description of Artificial Sequence Synthetic polynucleotide
  misc_feature (21)..(21): n is a, c, g, or t
ggcgggggccttgtcgttggngg

SEQ ID NO: 171 - DNA, 23 bp, Artificial Sequence
  Description of Artificial Sequence Synthetic polynucleotide
  misc_feature (21)..(21): n is a, c, g, or t
ctctggagttgtggtgggcgngg

SEQ ID NO: 172 - DNA, 23 bp, Artificial Sequence
  Description of Artificial Sequence Synthetic polynucleotide
  misc_feature (21)..(21): n is a, c, g, or t
tctggagttgtggtgggcggngg

SEQ ID NO: 173 - DNA, 23 bp, Artificial Sequence
  Description of Artificial Sequence Synthetic polynucleotide
  misc_feature (21)..(21): n is a, c, g, or t
cgttggaggtgttgtcttctngg

SEQ ID NO: 174 - DNA, 23 bp, Artificial Sequence
  Description of Artificial Sequence Synthetic polynucleotide
  misc_feature (21)..(21): n is a, c, g, or t
agacaacacctccaacgacangg

SEQ ID NO: 175 - DNA, 23 bp, Artificial Sequence
  Description of Artificial Sequence Synthetic polynucleotide
  misc_feature (21)..(21): n is a, c, g, or t
gtgggcgggggccttgtcgtngg

SEQ ID NO: 176 - DNA, 23 bp, Artificial Sequence
  Description of Artificial Sequence Synthetic polynucleotide
  misc_feature (21)..(21): n is a, c, g, or t
tcgttggaggtgttgtcttcngg

SEQ ID NO: 177 - DNA, 23 bp, Artificial Sequence
  Description of Artificial Sequence Synthetic polynucleotide
  misc_feature (21)..(21): n is a, c, g, or t
accacaactccagagcccacngg

SEQ ID NO: 178 - DNA, 23 bp, Artificial Sequence
  Description of Artificial Sequence Synthetic polynucleotide
  misc_feature (21)..(21): n is a, c, g, or t
gctctggagttgtggtgggcngg

SEQ ID NO: 179 - DNA, 23 bp, Artificial Sequence
  Description of Artificial Sequence Synthetic polynucleotide
  misc_feature (21)..(21): n is a, c, g, or t
gtgggctctggagttgtggtngg

SEQ ID NO: 180 - DNA, 23 bp, Artificial Sequence
  Description of Artificial Sequence Synthetic polynucleotide
  misc_feature (21)..(21): n is a, c, g, or t
tgtgggctctggagttgtggngg

SEQ ID NO: 181 - DNA, 23 bp, Artificial Sequence
  Description of Artificial Sequence Synthetic polynucleotide
  misc_feature (21)..(21): n is a, c, g, or t
gttggaggtgttgtcttctgngg

SEQ ID NO: 182 - DNA, 23 bp, Artificial Sequence
  Description of Artificial Sequence Synthetic polynucleotide
  misc_feature (21)..(21): n is a, c, g, or t
ggctctggagttgtggtgggngg

SEQ ID NO: 183 - DNA, 23 bp, Artificial Sequence
  Description of Artificial Sequence Synthetic polynucleotide
  misc_feature (21)..(21): n is a, c, g, or t
catagctgatggtaccccccngg

SEQ ID NO: 184 - DNA, 23 bp, Artificial Sequence
  Description of Artificial Sequence Synthetic polynucleotide
  misc_feature (21)..(21): n is a, c, g, or t
cggccagcactgtgccggcgngg

SEQ ID NO: 185 - DNA, 23 bp, Artificial Sequence
  Description of Artificial Sequence Synthetic polynucleotide
  misc_feature (21)..(21): n is a, c, g, or t
caagaactcggccacttttcngg

SEQ ID NO: 186 - DNA, 23 bp, Artificial Sequence
  Description of Artificial Sequence Synthetic polynucleotide
  misc_feature (21)..(21): n is a, c, g, or t
ggtgttcccgtggctcccctngg

SEQ ID NO: 187 - DNA, 23 bp, Artificial Sequence
  Description of Artificial Sequence Synthetic polynucleotide
  misc_feature (21)..(21): n is a, c, g, or t
ccagcactgtgccggcgtggngg

SEQ ID NO: 188 - DNA, 23 bp, Artificial Sequence
  Description of Artificial Sequence Synthetic polynucleotide
  misc_feature (21)..(21): n is a, c, g, or t
ggcaagggctggtgttcccgngg

SEQ ID NO: 189 - DNA, 23 bp, Artificial Sequence
  Description of Artificial Sequence Synthetic polynucleotide
  misc_feature (21)..(21): n is a, c, g, or t
ggcgtggtggagttctacagngg

SEQ ID NO: 190 - DNA, 23 bp, Artificial Sequence
  Description of Artificial Sequence Synthetic polynucleotide
  misc_feature (21)..(21): n is a, c, g, or t
ccaccacgccggcacagtgcngg

SEQ ID NO: 191 - DNA, 23 bp, Artificial Sequence
  Description of Artificial Sequence Synthetic polynucleotide
  misc_feature (21)..(21): n is a, c, g, or t
ggagttctacagcggcagccngg

SEQ ID NO: 192 - DNA, 23 bp, Artificial Sequence
  Description of Artificial Sequence Synthetic polynucleotide
  misc_feature (21)..(21): n is a, c, g, or t
gttctacagcggcagcctggngg

SEQ ID NO: 193 - DNA, 23 bp, Artificial Sequence
  Description of Artificial Sequence Synthetic polynucleotide
  misc_feature (21)..(21): n is a, c, g, or t
accagcccttgccaatccaangg

SEQ ID NO: 194 - DNA, 23 bp, Artificial Sequence
  Description of Artificial Sequence Synthetic polynucleotide
  misc_feature (21)..(21): n is a, c, g, or t
agttctacagcggcagcctgngg

SEQ ID NO: 195 - DNA, 23 bp, Artificial Sequence
  Description of Artificial Sequence Synthetic polynucleotide
  misc_feature (21)..(21): n is a, c, g, or t
ccaggtcctgggtcttgtccngg

SEQ ID NO: 196 - DNA, 23 bp, Artificial Sequence
  Description of Artificial Sequence Synthetic polynucleotide
  misc_feature (21)..(21): n is a, c, g, or t
tggtgttcccgtggctccccngg

SEQ ID NO: 197 - DNA, 23 bp, Artificial Sequence
  Description of Artificial Sequence Synthetic polynucleotide
  misc_feature (21)..(21): n is a, c, g, or t
gagttctacagcggcagcctngg

SEQ ID NO: 198 - DNA, 23 bp, Artificial Sequence
  Description of Artificial Sequence Synthetic polynucleotide
  misc_feature (21)..(21): n is a, c, g, or t
gaactcaagctgtacctcccngg

SEQ ID NO: 199 - DNA, 23 bp, Artificial Sequence
  Description of Artificial Sequence Synthetic polynucleotide
  misc_feature (21)..(21): n is a, c, g, or t
aagaactcggccacttttctngg

SEQ ID NO: 200 - DNA, 23 bp, Artificial Sequence
  Description of Artificial Sequence Synthetic polynucleotide
  misc_feature (21)..(21): n is a, c, g, or t
tccattggattggcaagggcngg

SEQ ID NO: 201 - DNA, 23 bp, Artificial Sequence
  Description of Artificial Sequence Synthetic polynucleotide
  misc_feature (21)..(21): n is a, c, g, or t
ttctacagcggcagcctgggngg

SEQ ID NO: 202 - DNA, 23 bp, Artificial Sequence
  Description of Artificial Sequence Synthetic polynucleotide
  misc_feature (21)..(21): n is a, c, g, or t
agaactcggccacttttctgngg

SEQ ID NO: 203 - DNA, 23 bp, Artificial Sequence
  Description of Artificial Sequence Synthetic polynucleotide
  misc_feature (21)..(21): n is a, c, g, or t
gcttcaagaaggagccacacngg

SEQ ID NO: 204 - DNA, 23 bp, Artificial Sequence
  Description of Artificial Sequence Synthetic polynucleotide
  misc_feature (21)..(21): n is a, c, g, or t
gatcttccattggattggcangg

SEQ ID NO: 205 - DNA, 23 bp, Artificial Sequence
  Description of Artificial Sequence Synthetic polynucleotide
  misc_feature (21)..(21): n is a, c, g, or t
gctgtagaactccaccacgcngg

SEQ ID NO: 206 - DNA, 23 bp, Artificial Sequence
  Description of Artificial Sequence Synthetic polynucleotide
  misc_feature (21)..(21): n is a, c, g, or t
gtcctgggcctcatagctgangg

SEQ ID NO: 207 - DNA, 23 bp, Artificial Sequence
  Description of Artificial Sequence Synthetic polynucleotide
  misc_feature (21)..(21): n is a, c, g, or t
taccatcagctatgaggcccngg

SEQ ID NO: 208 - DNA, 23 bp, Artificial Sequence
  Description of Artificial Sequence Synthetic polynucleotide
  misc_feature (21)..(21): n is a, c, g, or t
ggggggtaccatcagctatgngg

SEQ ID NO: 209 - DNA, 23 bp, Artificial Sequence
  Description of Artificial Sequence Synthetic polynucleotide
  misc_feature (21)..(21): n is a, c, g, or t
cctgaagcaatgctccagggngg

SEQ ID NO: 210 - DNA, 23 bp, Artificial Sequence
  Description of Artificial Sequence Synthetic polynucleotide
  misc_feature (21)..(21): n is a, c, g, or t
ttttcctgaagcaatgctccngg

SEQ ID NO: 211 - DNA, 23 bp, Artificial Sequence
  Description of Artificial Sequence Synthetic polynucleotide
  misc_feature (21)..(21): n is a, c, g, or t
ctctggcagatgcttcaagangg

SEQ ID NO: 212 - DNA, 23 bp, Artificial Sequence
  Description of Artificial Sequence Synthetic polynucleotide
  misc_feature (21)..(21): n is a, c, g, or t
agaggaagttctccaggtccngg

SEQ ID NO: 213 - DNA, 23 bp, Artificial Sequence
  Description of Artificial Sequence Synthetic polynucleotide
  misc_feature (21)..(21): n is a, c, g, or t
tctggcggccagcactgtgcngg

SEQ ID NO: 214 - DNA, 23 bp, Artificial Sequence
  Description of Artificial Sequence Synthetic polynucleotide
  misc_feature (21)..(21): n is a, c, g, or t
ttgagttctggatcttccatngg

SEQ ID NO: 215 - DNA, 23 bp, Artificial Sequence
  Description of Artificial Sequence Synthetic polynucleotide
  misc_feature (21)..(21): n is a, c, g, or t
ttctggatcttccattggatngg

SEQ ID NO: 216 - DNA, 23 bp, Artificial Sequence
  Description of Artificial Sequence Synthetic polynucleotide
  misc_feature (21)..(21): n is a, c, g, or t
atcttccattggattggcaangg

SEQ ID NO: 217 - DNA, 23 bp, Artificial Sequence
  Description of Artificial Sequence Synthetic polynucleotide
  misc_feature (21)..(21): n is a, c, g, or t
tcaagaaggagccacactggngg

SEQ ID NO: 218 - DNA, 23 bp, Artificial Sequence
  Description of Artificial Sequence Synthetic polynucleotide
  misc_feature (21)..(21): n is a, c, g, or t
gggaggtacagcttgagttcngg

SEQ ID NO: 219 - DNA, 23 bp, Artificial Sequence
  Description of Artificial Sequence Synthetic polynucleotide
  misc_feature (21)..(21): n is a, c, g, or t
cccgtggctcccctgggtctngg

SEQ ID NO: 220 - DNA, 23 bp, Artificial Sequence
  Description of Artificial Sequence Synthetic polynucleotide
  misc_feature (21)..(21): n is a, c, g, or t
ccaggacaagacccaggaccngg

SEQ ID NO: 221 - DNA, 23 bp, Artificial Sequence
  Description of Artificial Sequence Synthetic polynucleotide
  misc_feature (21)..(21): n is a, c, g, or t
ctctgcaacaacctccagtgngg

SEQ ID NO: 222 - DNA, 23 bp, Artificial Sequence
  Description of Artificial Sequence Synthetic polynucleotide
  misc_feature (21)..(21): n is a, c, g, or t
tgttgcagaggaagttctccngg

SEQ ID NO: 223 - DNA, 23 bp, Artificial Sequence
  Description of Artificial Sequence Synthetic polynucleotide
  misc_feature (21)..(21): n is a, c, g, or t
caggtcctgggtcttgtcctngg

SEQ ID NO: 224 - DNA, 23 bp, Artificial Sequence
  Description of Artificial Sequence Synthetic polynucleotide
  misc_feature (21)..(21): n is a, c, g, or t
tgaggcccaggacaagacccngg

SEQ ID NO: 225 - DNA, 23 bp, Artificial Sequence
  Description of Artificial Sequence Synthetic polynucleotide
  misc_feature (21)..(21): n is a, c, g, or t
ctgtgccaccagctgcagccngg

SEQ ID NO: 226 - DNA, 23 bp, Artificial Sequence
  Description of Artificial Sequence Synthetic polynucleotide
  misc_feature (21)..(21): n is a, c, g, or t
tgtgccaccagctgcagcctngg

SEQ ID NO: 227 - DNA, 23 bp, Artificial Sequence
  Description of Artificial Sequence Synthetic polynucleotide
  misc_feature (21)..(21): n is a, c, g, or t
catctgccagagactgaggcngg

SEQ ID NO: 228 - DNA, 23 bp, Artificial Sequence
  Description of Artificial Sequence Synthetic polynucleotide
  misc_feature (21)..(21): n is a, c, g, or t
ctgcagctggtggcacagtcngg

SEQ ID NO: 229 - DNA, 23 bp, Artificial Sequence
  Description of Artificial Sequence Synthetic polynucleotide
  misc_feature (21)..(21): n is a, c, g, or t
cacactggaggttgttgcagngg

SEQ ID NO: 230 - DNA, 23 bp, Artificial Sequence
  Description of Artificial Sequence Synthetic polynucleotide
  misc_feature (21)..(21): n is a, c, g, or t
cagctggtggcacagtctggngg

SEQ ID NO: 231 - DNA, 23 bp, Artificial Sequence
  Description of Artificial Sequence Synthetic polynucleotide
  misc_feature (21)..(21): n is a, c, g, or t
agcaaaggagggcaagaactngg

SEQ ID NO: 232 - DNA, 23 bp, Artificial Sequence
  Description of Artificial Sequence Synthetic polynucleotide
  misc_feature (21)..(21): n is a, c, g, or t
gaggaagttctccaggtcctngg

SEQ ID NO: 233 - DNA, 23 bp, Artificial Sequence
  Description of Artificial Sequence Synthetic polynucleotide
  misc_feature (21)..(21): n is a, c, g, or t
gccaccagctgcagcctgggngg

SEQ ID NO: 234 - DNA, 23 bp, Artificial Sequence
  Description of Artificial Sequence Synthetic polynucleotide
  misc_feature (21)..(21): n is a, c, g, or t
gcaggcagagcccaagacccngg

SEQ ID NO: 235 - DNA, 23 bp, Artificial Sequence
  Description of Artificial Sequence Synthetic polynucleotide
  misc_feature (21)..(21): n is a, c, g, or t
caggcagagcccaagacccangg

SEQ ID NO: 236 - DNA, 23 bp, Artificial Sequence
  Description of Artificial Sequence Synthetic polynucleotide
  misc_feature (21)..(21): n is a, c, g, or t
tcctcccaggctgcagctggngg

SEQ ID NO: 237 - DNA, 23 bp, Artificial Sequence
  Description of Artificial Sequence Synthetic polynucleotide
  misc_feature (21)..(21): n is a, c, g, or t
gctctgcctgcctcagtctcngg

SEQ ID NO: 238 - DNA, 23 bp, Artificial Sequence
  Description of Artificial Sequence Synthetic polynucleotide
  misc_feature (21)..(21): n is a, c, g, or t
cctccctggagcattgcttcngg

SEQ ID NO: 239 - DNA, 23 bp, Artificial Sequence
  Description of Artificial Sequence Synthetic polynucleotide
  misc_feature (21)..(21): n is a, c, g, or t
tttcctgaagcaatgctccangg

SEQ ID NO: 240 - DNA, 23 bp, Artificial Sequence
  Description of Artificial Sequence Synthetic polynucleotide
  misc_feature (21)..(21): n is a, c, g, or t
ccgtggctcccctgggtcttngg

SEQ ID NO: 241 - DNA, 23 bp, Artificial Sequence
  Description of Artificial Sequence Synthetic polynucleotide
  misc_feature (21)..(21): n is a, c, g, or t
aaaatcaagccccagaaaagngg

SEQ ID NO: 242 - DNA, 23 bp, Artificial Sequence
  Description of Artificial Sequence Synthetic polynucleotide
  misc_feature (21)..(21): n is a, c, g, or t
gaagcatctgccagagactgngg

SEQ ID NO: 243 - DNA, 23 bp, Artificial Sequence
  Description of Artificial Sequence Synthetic polynucleotide
  misc_feature (21)..(21): n is a, c, g, or t
gaagcatctgccagagactgngg

SEQ ID NO: 244 - DNA, 23 bp, Artificial Sequence
  Description of Artificial Sequence Synthetic polynucleotide
  misc_feature (21)..(21): n is a, c, g, or t
aggcagagcccaagacccagngg

SEQ ID NO: 245 - DNA, 23 bp, Artificial Sequence
  Description of Artificial Sequence Synthetic polynucleotide
  misc_feature (21)..(21): n is a, c, g, or t
cccaagacccaggggagccangg

SEQ ID NO: 246 - DNA, 23 bp, Artificial Sequence
  Description of Artificial Sequence Synthetic polynucleotide
  misc_feature (21)..(21): n is a, c, g, or t
tactcaggttcaggagacgcngg

SEQ ID NO: 247 - DNA, 23 bp, Artificial Sequence
  Description of Artificial Sequence Synthetic polynucleotide
  misc_feature (21)..(21): n is a, c, g, or t
tcaggagacgccgggcctccngg

SEQ ID NO: 248 - DNA, 23 bp, Artificial Sequence
  Description of Artificial Sequence Synthetic polynucleotide
  misc_feature (21)..(21): n is a, c, g, or t
actcaggttcaggagacgccngg

SEQ ID NO: 249 - DNA, 23 bp, Artificial Sequence
  Description of Artificial Sequence Synthetic polynucleotide
  misc_feature (21)..(21): n is a, c, g, or t
cagtgtctctactcaggttcngg

SEQ ID NO: 250 - DNA, 23 bp, Artificial Sequence
  Description of Artificial Sequence Synthetic polynucleotide
  misc_feature (21)..(21): n is a, c, g, or t
atgctcccagggctgcgtgcngg

SEQ ID NO: 251 - DNA, 23 bp, Artificial Sequence
  Description of Artificial Sequence Synthetic polynucleotide
  misc_feature (21)..(21): n is a, c, g, or t
gagacgccgggcctcctggangg

SEQ ID NO: 252 - DNA, 23 bp, Artificial Sequence
  Description of Artificial Sequence Synthetic polynucleotide
  misc_feature (21)..(21): n is a, c, g, or t
cagcagcagtgtctctactcngg

SEQ ID NO: 253 - DNA, 23 bp, Artificial Sequence
  Description of Artificial Sequence Synthetic polynucleotide
  misc_feature (21)..(21): n is a, c, g, or t
gatggcattcacatgctcccngg

SEQ ID NO: 254 - DNA, 23 bp, Artificial Sequence
  Description of Artificial Sequence Synthetic polynucleotide
  misc_feature (21)..(21): n is a, c, g, or t
ggagcatgtgaatgccatccngg

SEQ ID NO: 255 - DNA, 23 bp, Artificial Sequence
  Description of Artificial Sequence Synthetic polynucleotide
  misc_feature (21)..(21): n is a, c, g, or t
tagagacactgctgctgagangg

SEQ ID NO: 256 - DNA, 23 bp, Artificial Sequence
  Description of Artificial Sequence Synthetic polynucleotide
  misc_feature (21)..(21): n is a, c, g, or t
gcatgtgaatgccatccaggngg

SEQ ID NO: 257 - DNA, 23 bp, Artificial Sequence
  Description of Artificial Sequence Synthetic polynucleotide
  misc_feature (21)..(21): n is a, c, g, or t
atggcattcacatgctcccangg

SEQ ID NO: 258 - DNA, 23 bp, Artificial Sequence
  Description of Artificial Sequence Synthetic polynucleotide
  misc_feature (21)..(21): n is a, c, g, or t
tgaatgccatccaggaggccngg

SEQ ID NO: 259 - DNA, 23 bp, Artificial Sequence
  Description of Artificial Sequence Synthetic polynucleotide
  misc_feature (21)..(21): n is a, c, g, or t
tgctcccagggctgcgtgctngg

SEQ ID NO: 260 - DNA, 23 bp, Artificial Sequence
  Description of Artificial Sequence Synthetic polynucleotide
  misc_feature (21)..(21): n is a, c, g, or t
cagccccagcacgcagccctngg

SEQ ID NO: 261 - DNA, 23 bp, Artificial Sequence
  Description of Artificial Sequence Synthetic polynucleotide
  misc_feature (21)..(21): n is a, c, g, or t
gctcccagggctgcgtgctgngg

SEQ ID NO: 262 - DNA, 23 bp, Artificial Sequence
  Description of Artificial Sequence Synthetic polynucleotide
  misc_feature (21)..(21): n is a, c, g, or t
ccttggatttcagcggcacangg

SEQ ID NO: 263 - DNA, 23 bp, Artificial Sequence
  Description of Artificial Sequence Synthetic polynucleotide
  misc_feature (21)..(21): n is a, c, g, or t
ccttgtgccgctgaaatccangg

SEQ ID NO: 264 - DNA, 23 bp, Artificial Sequence
  Description of Artificial Sequence Synthetic polynucleotide
  misc_feature (21)..(21): n is a, c, g, or t
tgaacctggctaccaggaccngg

SEQ ID NO: 265 - DNA, 23 bp, Artificial Sequence
  Description of Artificial Sequence Synthetic polynucleotide
  misc_feature (21)..(21): n is a, c, g, or t
agggccaggtcctggtagccngg

SEQ ID NO: 266 - DNA, 23 bp, Artificial Sequence
  Description of Artificial Sequence Synthetic polynucleotide
  misc_feature (21)..(21): n is a, c, g, or t
ctcagctgaacctggctaccngg

SEQ ID NO: 267 - DNA, 23 bp, Artificial Sequence
  Description of Artificial Sequence Synthetic polynucleotide
  misc_feature (21)..(21): n is a, c, g, or t
agaaaaaacaggagagtgcangg

SEQ ID NO: 268 - DNA, 23 bp, Artificial Sequence
  Description of Artificial Sequence Synthetic polynucleotide
  misc_feature (21)..(21): n is a, c, g, or t
gcacaaggctcagctgaaccngg

SEQ ID NO: 269 - DNA, 23 bp, Artificial Sequence
  Description of Artificial Sequence Synthetic polynucleotide
  misc_feature (21)..(21): n is a, c, g, or t
tggcttgccttggatttcagngg

SEQ ID NO: 270 - DNA, 23 bp, Artificial Sequence
  Description of Artificial Sequence Synthetic polynucleotide
  misc_feature (21)..(21): n is a, c, g, or t
aaacaggagagtgcagggccngg

SEQ ID NO: 271 - DNA, 23 bp, Artificial Sequence
  Description of Artificial Sequence Synthetic polynucleotide
  misc_feature (21)..(21): n is a, c, g, or t
gagagtgcagggccaggtccngg

SEQ ID NO: 272 - DNA, 23 bp, Artificial Sequence
  Description of Artificial Sequence Synthetic polynucleotide
  misc_feature (21)..(21): n is a, c, g, or t
ggatgaagagaagaaaaaacngg

SEQ ID NO: 273 - DNA, 23 bp, Artificial Sequence
  Description of Artificial Sequence Synthetic polynucleotide
  misc_feature (21)..(21): n is a, c, g, or t
aagaaaaaacaggagagtgcngg

SEQ ID NO: 274 - DNA, 23 bp, Artificial Sequence
  Description of Artificial Sequence Synthetic polynucleotide
  misc_feature (21)..(21): n is a, c, g, or t
ccgggtgacagtgcttcggcngg

SEQ ID NO: 275 - DNA, 23 bp, Artificial Sequence
  Description of Artificial Sequence Synthetic polynucleotide
  misc_feature (21)..(21): n is a, c, g, or t
acacaaagctggcgatgcctngg

SEQ ID NO: 276 - DNA, 23 bp, Artificial Sequence
  Description of Artificial Sequence Synthetic polynucleotide
  misc_feature (21)..(21): n is a, c, g, or t
ccctcagtccttggatagtgngg

SEQ ID NO: 277 - DNA, 23 bp, Artificial Sequence
  Description of Artificial Sequence Synthetic polynucleotide
  misc_feature (21)..(21): n is a, c, g, or t
gtgcggcaacctacatgatgngg

SEQ ID NO: 278 - DNA, 23 bp, Artificial Sequence
  Description of Artificial Sequence Synthetic polynucleotide
  misc_feature (21)..(21): n is a, c, g, or t
ctgtgcggcaacctacatgangg

SEQ ID NO: 279 - DNA, 23 bp, Artificial Sequence
  Description of Artificial Sequence Synthetic polynucleotide
  misc_feature (21)..(21): n is a, c, g, or t
ggcccagcctgctgtggtacngg

SEQ ID NO: 280 - DNA, 23 bp, Artificial Sequence
  Description of Artificial Sequence Synthetic polynucleotide
  misc_feature (21)..(21): n is a, c, g, or t
gttcacttgatttccactggngg

SEQ ID NO: 281 - DNA, 23 bp, Artificial Sequence
  Description of Artificial Sequence Synthetic polynucleotide
  misc_feature (21)..(21): n is a, c, g, or t
caactcattccccatcatgtngg

SEQ ID NO: 282 - DNA, 23 bp, Artificial Sequence
  Description of Artificial Sequence Synthetic polynucleotide
  misc_feature (21)..(21): n is a, c, g, or t
ccgcacagacttcagtcaccngg

SEQ ID NO: 283 - DNA, 23 bp, Artificial Sequence
  Description of Artificial Sequence Synthetic polynucleotide
  misc_feature (21)..(21): n is a, c, g, or t
tgtgcggcaacctacatgatngg

SEQ ID NO: 284 - DNA, 23 bp, Artificial Sequence
  Description of Artificial Sequence Synthetic polynucleotide
  misc_feature (21)..(21): n is a, c, g, or t
cctcactatccaaggactgangg

SEQ ID NO: 285 - DNA, 23 bp, Artificial Sequence
  Description of Artificial Sequence Synthetic polynucleotide
  misc_feature (21)..(21): n is a, c, g, or t
cggacctcagtggctttgccngg

SEQ ID NO: 286 - DNA, 23 bp, Artificial Sequence
  Description of Artificial Sequence Synthetic polynucleotide
  misc_feature (21)..(21): n is a, c, g, or t
gaggttcacttgatttccacngg

SEQ ID NO: 287 - DNA, 23 bp, Artificial Sequence
  Description of Artificial Sequence Synthetic polynucleotide
  misc_feature (21)..(21): n is a, c, g, or t
ccaggtgactgaagtctgtgngg

SEQ ID NO: 288 - DNA, 23 bp, Artificial Sequence
  Description of Artificial Sequence Synthetic polynucleotide
  misc_feature (21)..(21): n is a, c, g, or t
actggaggtgcccgtgcagangg

SEQ ID NO: 289 - DNA, 23 bp, Artificial Sequence
  Description of Artificial Sequence Synthetic polynucleotide
  misc_feature (21)..(21): n is a, c, g, or t
caagtgaacctcactatccangg

SEQ ID NO: 290 - DNA, 23 bp, Artificial Sequence
  Description of Artificial Sequence Synthetic polynucleotide
  misc_feature (21)..(21): n is a, c, g, or t
gtggtactggccagcagccgngg

SEQ ID NO: 291 - DNA, 23 bp, Artificial Sequence
  Description of Artificial Sequence Synthetic polynucleotide
  misc_feature (21)..(21): n is a, c, g, or t
aggtccgggtgacagtgcttngg

SEQ ID NO: 292 - DNA, 23 bp, Artificial Sequence
  Description of Artificial Sequence Synthetic polynucleotide
  misc_feature (21)..(21): n is a, c, g, or t
atctgcacgggcacctccagngg

SEQ ID NO: 293 - DNA, 23 bp, Artificial Sequence
  Description of Artificial Sequence Synthetic polynucleotide
  misc_feature (21)..(21): n is a, c, g, or t
ccgtgcagatggaatcatctngg

SEQ ID NO: 294 - DNA, 23 bp, Artificial Sequence
  Description of Artificial Sequence Synthetic polynucleotide
  misc_feature (21)..(21): n is a, c, g, or t
ctagatgattccatctgcacngg

SEQ ID NO: 295 - DNA, 23 bp, Artificial Sequence
  Description of Artificial Sequence Synthetic polynucleotide
  misc_feature (21)..(21): n is a, c, g, or t
acctcactatccaaggactgngg

SEQ ID NO: 296 - DNA, 23 bp, Artificial Sequence
  Description of Artificial Sequence Synthetic polynucleotide
  misc_feature (21)..(21): n is a, c, g, or t
cctgccgaagcactgtcaccngg

SEQ ID NO: 297 - DNA, 23 bp, Artificial Sequence
  Description of Artificial Sequence Synthetic polynucleotide
  misc_feature (21)..(21): n is a, c, g, or t
tggccagtaccacagcaggcngg

SEQ ID NO: 298 - DNA, 23 bp, Artificial Sequence
  Description of Artificial Sequence Synthetic polynucleotide
  misc_feature (21)..(21): n is a, c, g, or t
atctccaggcaaagccactgngg

SEQ ID NO: 299 - DNA, 23 bp, Artificial Sequence
  Description of Artificial Sequence Synthetic polynucleotide
  misc_feature (21)..(21): n is a, c, g, or t
gcacgtggcccagcctgctgngg

SEQ ID NO: 300 - DNA, 23 bp, Artificial Sequence
  Description of Artificial Sequence Synthetic polynucleotide
  misc_feature (21)..(21): n is a, c, g, or t
gtgtgtgagtatgcatctccngg

SEQ ID NO: 301 - DNA, 23 bp, Artificial Sequence
  Description of Artificial Sequence Synthetic polynucleotide
  misc_feature (21)..(21): n is a, c, g, or t
cactgtcacccggacctcagngg

SEQ ID NO: 302 - DNA, 23 bp, Artificial Sequence
  Description of Artificial Sequence Synthetic polynucleotide
  misc_feature (21)..(21): n is a, c, g, or t
gctggcgatgcctcggctgcngg

SEQ ID NO: 303 - DNA, 23 bp, Artificial Sequence
  Description of Artificial Sequence Synthetic polynucleotide
  misc_feature (21)..(21): n is a, c, g, or t
ctgctggccagtaccacagcngg

SEQ ID NO: 304 - DNA, 23 bp, Artificial Sequence
  Description of Artificial Sequence Synthetic polynucleotide
  misc_feature (21)..(21): n is a, c, g, or t
aggcaaagccactgaggtccngg

SEQ ID NO: 305 - DNA, 23 bp, Artificial Sequence
  Description of Artificial Sequence Synthetic polynucleotide
  misc_feature (21)..(21): n is a, c, g, or t
gcagatggaatcatctaggangg

SEQ ID NO: 306 - DNA, 23 bp, Artificial Sequence
  Description of Artificial Sequence Synthetic polynucleotide
  misc_feature (21)..(21): n is a, c, g, or t
cctagatgattccatctgcangg

SEQ ID NO: 307 - DNA, 23 bp, Artificial Sequence
  Description of Artificial Sequence Synthetic polynucleotide
  misc_feature (21)..(21): n is a, c, g, or t
ggccagtaccacagcaggctngg

SEQ ID NO: 308 - DNA, 23 bp, Artificial Sequence
  Description of Artificial Sequence Synthetic polynucleotide
  misc_feature (21)..(21): n is a, c, g, or t
tgcatactcacacacaaagcngg

SEQ ID NO: 309 - DNA, 23 bp, Artificial Sequence
  Description of Artificial Sequence Synthetic polynucleotide
  misc_feature (21)..(21): n is a, c, g, or t
gcttcggcaggctgacagccngg

SEQ ID NO: 310 - DNA, 23 bp, Artificial Sequence
  Description of Artificial Sequence Synthetic polynucleotide
  misc_feature (21)..(21): n is a, c, g, or t
caggcaaagccactgaggtcngg

SEQ ID NO: 311 - DNA, 23 bp, Artificial Sequence
  Description of Artificial Sequence Synthetic polynucleotide
  misc_feature (21)..(21): n is a, c, g, or t
tgtagcaccgcccagacgacngg

SEQ ID NO: 312 - DNA, 23 bp, Artificial Sequence
  Description of Artificial Sequence Synthetic polynucleotide
  misc_feature (21)..(21): n is a, c, g, or t
ggcgccctggccagtcgtctngg

SEQ ID NO: 313 - DNA, 23 bp, Artificial Sequence
  Description of Artificial Sequence Synthetic polynucleotide
  misc_feature (21)..(21): n is a, c, g, or t
cgtctgggcggtgctacaacngg

SEQ ID NO: 314 - DNA, 23 bp, Artificial Sequence
  Description of Artificial Sequence Synthetic polynucleotide
  misc_feature (21)..(21): n is a, c, g, or t
aggcgccctggccagtcgtcngg

SEQ ID NO: 315 - DNA, 23 bp, Artificial Sequence
  Description of Artificial Sequence Synthetic polynucleotide
  misc_feature (21)..(21): n is a, c, g, or t
caccgcccagacgactggccngg

SEQ ID NO: 316 - DNA, 23 bp, Artificial Sequence
  Description of Artificial Sequence Synthetic polynucleotide
  misc_feature (21)..(21): n is a, c, g, or t
accgcccagacgactggccangg

SEQ ID NO: 317 - DNA, 23 bp, Artificial Sequence
  Description of Artificial Sequence Synthetic polynucleotide
  misc_feature (21)..(21): n is a, c, g, or t
gggcggtgctacaactgggcngg

SEQ ID NO: 318 - DNA, 23 bp, Artificial Sequence
  Description of Artificial Sequence Synthetic polynucleotide
  misc_feature (21)..(21): n is a, c, g, or t
gtctgggcggtgctacaactngg

SEQ ID NO: 319 - DNA, 23 bp, Artificial Sequence
  Description of Artificial Sequence Synthetic polynucleotide
  misc_feature (21)..(21): n is a, c, g, or t
cgactggccagggcgcctgtngg

SEQ ID NO: 320 - DNA, 23 bp, Artificial Sequence
  Description of Artificial Sequence Synthetic polynucleotide
  misc_feature (21)..(21): n is a, c, g, or t
cggtgctacaactgggctggngg

SEQ ID NO: 321 - DNA, 23 bp, Artificial Sequence
  Description of Artificial Sequence Synthetic polynucleotide
  misc_feature (21)..(21): n is a, c, g, or t
tggcggccaggatggttcttngg

SEQ ID NO: 322 - DNA, 23 bp, Artificial Sequence
  Description of Artificial Sequence Synthetic polynucleotide
  misc_feature (21)..(21): n is a, c, g, or t
acgactggccagggcgcctgngg

SEQ ID NO: 323 - DNA, 23 bp, Artificial Sequence
  Description of Artificial Sequence Synthetic polynucleotide
  misc_feature (21)..(21): n is a, c, g, or t
ctacaactgggctggcggccngg

SEQ ID NO: 324 - DNA, 23 bp, Artificial Sequence
  Description of Artificial Sequence Synthetic polynucleotide
  misc_feature (21)..(21): n is a, c, g, or t
gccctggccagtcgtctgggngg

SEQ ID NO: 325 - DNA, 23 bp, Artificial Sequence
  Description of Artificial Sequence Synthetic polynucleotide
  misc_feature (21)..(21): n is a, c, g, or t
tgcagatcccacaggcgcccngg

SEQ ID NO: 326 - DNA, 23 bp, Artificial Sequence
  Description of Artificial Sequence Synthetic polynucleotide
  misc_feature (21)..(21): n is a, c, g, or t
aactgggctggcggccaggangg

SEQ ID NO: 327 - DNA, 23 bp, Artificial Sequence
  Description of Artificial Sequence Synthetic polynucleotide
  misc_feature (21)..(21): n is a, c, g, or t
cggagagcttcgtgctaaacngg

SEQ ID NO: 328 - DNA, 23 bp, Artificial Sequence
  Description of Artificial Sequence Synthetic polynucleotide
  misc_feature (21)..(21): n is a, c, g, or t
gcgtgacttccacatgagcgngg

SEQ ID NO: 329 - DNA, 23 bp, Artificial Sequence
  Description of Artificial Sequence Synthetic polynucleotide
  misc_feature (21)..(21): n is a, c, g, or t
atgtggaagtcacgcccgttngg

SEQ ID NO: 330 - DNA, 23 bp, Artificial Sequence
  Description of Artificial Sequence Synthetic polynucleotide
  misc_feature (21)..(21): n is a, c, g, or t
gccctgctcgtggtgaccgangg

SEQ ID NO: 331 - DNA, 23 bp, Artificial Sequence
  Description of Artificial Sequence Synthetic polynucleotide
  misc_feature (21)..(21): n is a, c, g, or t
cacgaagctctccgatgtgtngg

SEQ ID NO: 332 - DNA, 23 bp, Artificial Sequence
  Description of Artificial Sequence Synthetic polynucleotide
  misc_feature (21)..(21): n is a, c, g, or t
cctgctcgtggtgaccgaagngg

SEQ ID NO: 333 - DNA, 23 bp, Artificial Sequence
  Description of Artificial Sequence Synthetic polynucleotide
  misc_feature (21)..(21): n is a, c, g, or t
tgacacggaagcggcagtccngg

SEQ ID NO: 334 - DNA, 23 bp, Artificial Sequence
  Description of Artificial Sequence Synthetic polynucleotide
  misc_feature (21)..(21): n is a, c, g, or t
ccccttcggtcaccacgagcngg

SEQ ID NO: 335 - DNA, 23 bp, Artificial Sequence
  Description of Artificial Sequence Synthetic polynucleotide
  misc_feature (21)..(21): n is a, c, g, or t
cagcaaccagacggacaagcngg

SEQ ID NO: 336 - DNA, 23 bp, Artificial Sequence
  Description of Artificial Sequence Synthetic polynucleotide
  misc_feature (21)..(21): n is a, c, g, or t
gcagttgtgtgacacggaagngg

SEQ ID NO: 337 - DNA, 23 bp, Artificial Sequence
  Description of Artificial Sequence Synthetic polynucleotide
  misc_feature (21)..(21): n is a, c, g, or t
cccttcggtcaccacgagcangg

SEQ ID NO: 338 - DNA, 23 bp, Artificial Sequence
  Description of Artificial Sequence Synthetic polynucleotide
  misc_feature (21)..(21): n is a, c, g, or t
ccgggctggctgcggtcctcngg

SEQ ID NO: 339 - DNA, 23 bp, Artificial Sequence
  Description of Artificial Sequence Synthetic polynucleotide
  misc_feature (21)..(21): n is a, c, g, or t
aggcggccagcttgtccgtcngg

SEQ ID NO: 340 - DNA, 23 bp, Artificial Sequence
  Description of Artificial Sequence Synthetic polynucleotide
  misc_feature (21)..(21): n is a, c, g, or t
cagcttgtccgtctggttgcngg

SEQ ID NO: 341 - DNA, 23 bp, Artificial Sequence
  Description of Artificial Sequence Synthetic polynucleotide
  misc_feature (21)..(21): n is a, c, g, or t
cggtcaccacgagcagggctngg

SEQ ID NO: 342 - DNA, 23 bp, Artificial Sequence
  Description of Artificial Sequence Synthetic polynucleotide
  misc_feature (21)..(21): n is a, c, g, or t
gtgtcacacaactgcccaacngg

SEQ ID NO: 343 - DNA, 23 bp, Artificial Sequence
  Description of Artificial Sequence Synthetic polynucleotide
  misc_feature (21)..(21): n is a, c, g, or t
ctgcagcttctccaacacatngg

SEQ ID NO: 344 - DNA, 23 bp, Artificial Sequence
  Description of Artificial Sequence Synthetic polynucleotide
  misc_feature (21)..(21): n is a, c, g, or t
caagctggccgccttccccgngg

SEQ ID NO: 345 - DNA, 23 bp, Artificial Sequence
  Description of Artificial Sequence Synthetic polynucleotide
  misc_feature (21)..(21): n is a, c, g, or t
cgtgtcacacaactgcccaangg

SEQ ID NO: 346 - DNA, 23 bp, Artificial Sequence
  Description of Artificial Sequence Synthetic polynucleotide
  misc_feature (21)..(21): n is a, c, g, or t
cgttgggcagttgtgtgacangg

SEQ ID NO: 347 - DNA, 23 bp, Artificial Sequence
  Description of Artificial Sequence Synthetic polynucleotide
  misc_feature (21)..(21): n is a, c, g, or t
gcttgtccgtctggttgctgngg

SEQ ID NO: 348 - DNA, 23 bp, Artificial Sequence
  Description of Artificial Sequence Synthetic polynucleotide
  misc_feature (21)..(21): n is a, c, g, or t
cggaagcggcagtcctggccngg

SEQ ID NO: 349 - DNA, 23 bp, Artificial Sequence
  Description of Artificial Sequence Synthetic polynucleotide
  misc_feature (21)..(21): n is a, c, g, or t
cgatgtgttggagaagctgcngg

SEQ ID NO: 350 - DNA, 23 bp, Artificial Sequence
  Description of Artificial Sequence Synthetic polynucleotide
  misc_feature (21)..(21): n is a, c, g, or t
catgtggaagtcacgcccgtngg

SEQ ID NO: 351 - DNA, 23 bp, Artificial Sequence
  Description of Artificial Sequence Synthetic polynucleotide
  misc_feature (21)..(21): n is a, c, g, or t
ccctgctcgtggtgaccgaangg

SEQ ID NO: 352 - DNA, 23 bp, Artificial Sequence
  Description of Artificial Sequence Synthetic polynucleotide
  misc_feature (21)..(21): n is a, c, g, or t
cgggctggctgcggtcctcgngg

SEQ ID NO: 353 - DNA, 23 bp, Artificial Sequence
  Description of Artificial Sequence Synthetic polynucleotide
  misc_feature (21)..(21): n is a, c, g, or t
agcttgtccgtctggttgctngg

SEQ ID NO: 354 - DNA, 23 bp, Artificial Sequence
  Description of Artificial Sequence Synthetic polynucleotide
  misc_feature (21)..(21): n is a, c, g, or t
gaaggtggcgttgtccccttngg

SEQ ID NO: 355 - DNA, 23 bp, Artificial Sequence
  Description of Artificial Sequence Synthetic polynucleotide
  misc_feature (21)..(21): n is a, c, g, or t
acttccacatgagcgtggtcngg

SEQ ID NO: 356 - DNA, 23 bp, Artificial Sequence
  Description of Artificial Sequence Synthetic polynucleotide
  misc_feature (21)..(21): n is a, c, g, or t
gccgggctggctgcggtcctngg

SEQ ID NO: 357 - DNA, 23 bp, Artificial Sequence
  Description of Artificial Sequence Synthetic polynucleotide
  misc_feature (21)..(21): n is a, c, g, or t
tcggtcaccacgagcagggcngg

SEQ ID NO: 358 - DNA, 23 bp, Artificial Sequence
  Description of Artificial Sequence Synthetic polynucleotide
  misc_feature (21)..(21): n is a, c, g, or t
tctggttgctggggctcatgngg

SEQ ID NO: 359 - DNA, 23 bp, Artificial Sequence
  Description of Artificial Sequence Synthetic polynucleotide
  misc_feature (21)..(21): n is a, c, g, or t
acggaagcggcagtcctggcngg

SEQ ID NO: 360 - DNA, 23 bp, Artificial Sequence
  Description of Artificial Sequence Synthetic polynucleotide
  misc_feature (21)..(21): n is a, c, g, or t
cccgaggaccgcagccagccngg

SEQ ID NO: 361 - DNA, 23 bp, Artificial Sequence
  Description of Artificial Sequence Synthetic polynucleotide
  misc_feature (21)..(21): n is a, c, g, or t
ctggctgcggtcctcggggangg

SEQ ID NO: 362 - DNA, 23 bp, Artificial Sequence
  Description of Artificial Sequence Synthetic polynucleotide
  misc_feature (21)..(21): n is a, c, g, or t
catgagccccagcaaccagangg

SEQ ID NO: 363 - DNA, 23 bp, Artificial Sequence
  Description of Artificial Sequence Synthetic polynucleotide
  misc_feature (21)..(21): n is a, c, g, or t
agtcctggccgggctggctgngg

SEQ ID NO: 364 - DNA, 23 bp, Artificial Sequence
  Description of Artificial Sequence Synthetic polynucleotide
  misc_feature (21)..(21): n is a, c, g, or t
gggggttccagggcctgtctngg

SEQ ID NO: 365 - DNA, 23 bp, Artificial Sequence
  Description of Artificial Sequence Synthetic polynucleotide
  misc_feature (21)..(21): n is a, c, g, or t
ggtcaccacgagcagggctgngg

SEQ ID NO: 366 - DNA, 23 bp, Artificial Sequence
  Description of Artificial Sequence Synthetic polynucleotide
  misc_feature (21)..(21): n is a, c, g, or t
gctgcggtcctcggggaaggngg

SEQ ID NO: 367 - DNA, 23 bp, Artificial Sequence
  Description of Artificial Sequence Synthetic polynucleotide
  misc_feature (21)..(21): n is a, c, g, or t
ggaccgcagccagcccggccngg

SEQ ID NO: 368 - DNA, 23 bp, Artificial Sequence
  Description of Artificial Sequence Synthetic polynucleotide
  misc_feature (21)..(21): n is a, c, g, or t
gagaaggtgggggggttccangg

SEQ ID NO: 369 - DNA, 23 bp, Artificial Sequence
  Description of Artificial Sequence Synthetic polynucleotide
  misc_feature (21)..(21): n is a, c, g, or t
ggagaaggtgggggggttccngg

SEQ ID NO: 370 - DNA, 23 bp, Artificial Sequence
  Description of Artificial Sequence Synthetic polynucleotide
  misc_feature (21)..(21): n is a, c, g, or t
agcggcagtcctggccgggcngg

SEQ ID NO: 371 - DNA, 23 bp, Artificial Sequence
  Description of Artificial Sequence Synthetic polynucleotide
  misc_feature (21)..(21): n is a, c, g, or t
ggggttccagggcctgtctgngg

SEQ ID NO: 372 - DNA, 23 bp, Artificial Sequence
  Description of Artificial Sequence Synthetic polynucleotide
  misc_feature (21)..(21): n is a, c, g, or t
cttctccccagccctgctcgngg

SEQ ID NO: 373 - DNA, 23 bp, Artificial Sequence
  Description of Artificial Sequence Synthetic polynucleotide
  misc_feature (21)..(21): n is a, c, g, or t
gttggagaagctgcaggtgangg

SEQ ID NO: 374 - DNA, 23 bp, Artificial Sequence
  Description of Artificial Sequence Synthetic polynucleotide
  misc_feature (21)..(21): n is a, c, g, or t
ggggggttccagggcctgtcngg

SEQ ID NO: 375 - DNA, 23 bp, Artificial Sequence
  Description of Artificial Sequence Synthetic polynucleotide
  misc_feature (21)..(21): n is a, c, g, or t
ggagaagctgcaggtgaaggngg

SEQ ID NO: 376 - DNA, 23 bp, Artificial Sequence
  Description of Artificial Sequence Synthetic polynucleotide
  misc_feature (21)..(21): n is a, c, g, or t
cacgagcagggctggggagangg

SEQ ID NO: 377 - DNA, 23 bp, Artificial Sequence
  Description of Artificial Sequence Synthetic polynucleotide
  misc_feature (21)..(21): n is a, c, g, or t
gcagggctggggagaaggtgngg

SEQ ID NO: 378 - DNA, 23 bp, Artificial Sequence
  Description of Artificial Sequence Synthetic polynucleotide
  misc_feature (21)..(21): n is a, c, g, or t
cagggctggggagaaggtggngg

SEQ ID NO: 379 - DNA, 23 bp, Artificial Sequence
  Description of Artificial Sequence Synthetic polynucleotide
  misc_feature (21)..(21): n is a, c, g, or t
gagcagggctggggagaaggngg

SEQ ID NO: 380 - DNA, 23 bp, Artificial Sequence
  Description of Artificial Sequence Synthetic polynucleotide
  misc_feature (21)..(21): n is a, c, g, or t
agcagggctggggagaaggtngg

SEQ ID NO: 381 - DNA, 23 bp, Artificial Sequence
  Description of Artificial Sequence Synthetic polynucleotide
  misc_feature (21)..(21): n is a, c, g, or t
agggctggggagaaggtgggngg

SEQ ID NO: 382 - DNA, 23 bp, Artificial Sequence
  Description of Artificial Sequence Synthetic polynucleotide
  misc_feature (21)..(21): n is a, c, g, or t
gggctggggagaaggtggggngg

SEQ ID NO: 383 - DNA, 23 bp, Artificial Sequence
  Description of Artificial Sequence Synthetic polynucleotide
  misc_feature (21)..(21): n is a, c, g, or t
agaagtggaatacagagcggngg

SEQ ID NO: 384 - DNA, 23 bp, Artificial Sequence
  Description of Artificial Sequence Synthetic polynucleotide
  misc_feature (21)..(21): n is a, c, g, or t
aatgtggcaacgtggtgctcngg

SEQ ID NO: 385 - DNA, 23 bp, Artificial Sequence
  Description of Artificial Sequence Synthetic polynucleotide
  misc_feature (21)..(21): n is a, c, g, or t
ctaaatggggatttccgcaangg

SEQ ID NO: 386 - DNA, 23 bp, Artificial Sequence
  Description of Artificial Sequence Synthetic polynucleotide
  misc_feature (21)..(21): n is a, c, g, or t
catccagatactggctaaatngg

SEQ ID NO: 387 - DNA, 23 bp, Artificial Sequence
  Description of Artificial Sequence Synthetic polynucleotide
  misc_feature (21)..(21): n is a, c, g, or t
cagacgggcacgaggttcccngg

SEQ ID NO: 388 - DNA, 23 bp, Artificial Sequence
  Description of Artificial Sequence Synthetic polynucleotide
  misc_feature (21)..(21): n is a, c, g, or t
gcggctggggtgtagaagcangg

SEQ ID NO: 389 - DNA, 23 bp, Artificial Sequence
  Description of Artificial Sequence Synthetic polynucleotide
  misc_feature (21)..(21): n is a, c, g, or t
gaacctcgtgcccgtctgctngg

SEQ ID NO: 390 - DNA, 23 bp, Artificial Sequence
  Description of Artificial Sequence Synthetic polynucleotide
  misc_feature (21)..(21): n is a, c, g, or t
gacgggcacgaggttccctgngg

SEQ ID NO: 391 - DNA, 23 bp, Artificial Sequence
  Description of Artificial Sequence Synthetic polynucleotide
  misc_feature (21)..(21): n is a, c, g, or t
atccccatttagccagtatcngg

SEQ ID NO: 392 - DNA, 23 bp, Artificial Sequence
  Description of Artificial Sequence Synthetic polynucleotide
  misc_feature (21)..(21): n is a, c, g, or t
gtggaatacagagcggaggtngg

SEQ ID NO: 393 - DNA, 23 bp, Artificial Sequence
  Description of Artificial Sequence Synthetic polynucleotide
  misc_feature (21)..(21): n is a, c, g, or t
agacgggcacgaggttccctngg

SEQ ID NO: 394 - DNA, 23 bp, Artificial Sequence
  Description of Artificial Sequence Synthetic polynucleotide
  misc_feature (21)..(21): n is a, c, g, or t
ggaacctcgtgcccgtctgcngg

SEQ ID NO: 395 - DNA, 23 bp, Artificial Sequence
  Description of Artificial Sequence Synthetic polynucleotide
  misc_feature (21)..(21): n is a, c, g, or t
gagtcacattctctatggtcngg

SEQ ID NO: 396 - DNA, 23 bp, Artificial Sequence
  Description of Artificial Sequence Synthetic polynucleotide
  misc_feature (21)..(21): n is a, c, g, or t
atgtgactctagcagacagtngg

SEQ ID NO: 397 - DNA, 23 bp, Artificial Sequence
  Description of Artificial Sequence Synthetic polynucleotide
  misc_feature (21)..(21): n is a, c, g, or t
ttttcatcattcattatgccngg

SEQ ID NO: 398 - DNA, 23 bp, Artificial Sequence
  Description of Artificial Sequence Synthetic polynucleotide
  misc_feature (21)..(21): n is a, c, g, or t
aatgtgactctagcagacagngg

SEQ ID NO: 399 - DNA, 23 bp, Artificial Sequence
  Description of Artificial Sequence Synthetic polynucleotide
  misc_feature (21)..(21): n is a, c, g, or t
atccagatactggctaaatgngg

SEQ ID NO: 400 - DNA, 23 bp, Artificial Sequence
  Description of Artificial Sequence Synthetic polynucleotide
  misc_feature (21)..(21): n is a, c, g, or t
tgctgccggatccaaatcccngg

SEQ ID NO: 401 - DNA, 23 bp, Artificial Sequence
  Description of Artificial Sequence Synthetic polynucleotide
  misc_feature (21)..(21): n is a, c, g, or t
tctacaccccagccgccccangg

SEQ ID NO: 402 - DNA, 23 bp, Artificial Sequence
  Description of Artificial Sequence Synthetic polynucleotide
  misc_feature (21)..(21): n is a, c, g, or t
ttatgcctgggatttggatcngg

SEQ ID NO: 403 - DNA, 23 bp, Artificial Sequence
  Description of Artificial Sequence Synthetic polynucleotide
  misc_feature (21)..(21): n is a, c, g, or t
cgctctgtattccacttctgngg

SEQ ID NO: 404 - DNA, 23 bp, Artificial Sequence
  Description of Artificial Sequence Synthetic polynucleotide
  misc_feature (21)..(21): n is a, c, g, or t
gaggttccctggggcggctgngg

SEQ ID NO: 405 - DNA, 23 bp, Artificial Sequence
  Description of Artificial Sequence Synthetic polynucleotide
  misc_feature (21)..(21): n is a, c, g, or t
tgccccagcagacgggcacgngg

SEQ ID NO: 406 - DNA, 23 bp, Artificial Sequence
  Description of Artificial Sequence Synthetic polynucleotide
  misc_feature (21)..(21): n is a, c, g, or t
acagtgggatctactgctgcngg

SEQ ID NO: 407 - DNA, 23 bp, Artificial Sequence
  Description of Artificial Sequence Synthetic polynucleotide
  misc_feature (21)..(21): n is a, c, g, or t
tgtgtttgaatgtggcaacgngg

SEQ ID NO: 408 - DNA, 23 bp, Artificial Sequence
  Description of Artificial Sequence Synthetic polynucleotide
  misc_feature (21)..(21): n is a, c, g, or t
tgaaaaatttaacctgaagtngg

SEQ ID NO: 409 - DNA, 23 bp, Artificial Sequence
  Description of Artificial Sequence Synthetic polynucleotide
  misc_feature (21)..(21): n is a, c, g, or t
acatccagatactggctaaangg

SEQ ID NO: 410 - DNA, 23 bp, Artificial Sequence
  Description of Artificial Sequence Synthetic polynucleotide
  misc_feature (21)..(21): n is a, c, g, or t
atgaaagggatgtgaattatngg

SEQ ID NO: 411 - DNA, 23 bp, Artificial Sequence
  Description of Artificial Sequence Synthetic polynucleotide
  misc_feature (21)..(21): n is a, c, g, or t
tggtgctcaggactgatgaangg

SEQ ID NO: 412 - DNA, 23 bp, Artificial Sequence
  Description of Artificial Sequence Synthetic polynucleotide
  misc_feature (21)..(21): n is a, c, g, or t
ggtgtagaagcagggcagatngg

SEQ ID NO: 413 - DNA, 23 bp, Artificial Sequence
  Description of Artificial Sequence Synthetic polynucleotide
  misc_feature (21)..(21): n is a, c, g, or t
acgttgccacattcaaacacngg

SEQ ID NO: 414 - DNA, 23 bp, Artificial Sequence
  Description of Artificial Sequence Synthetic polynucleotide
  misc_feature (21)..(21): n is a, c, g, or t
acgaggttccctggggcggcngg

SEQ ID NO: 415 - DNA, 23 bp, Artificial Sequence
  Description of Artificial Sequence Synthetic polynucleotide
  misc_feature (21)..(21): n is a, c, g, or t
gcctgtcctgtgtttgaatgngg

SEQ ID NO: 416 - DNA, 23 bp, Artificial Sequence
  Description of Artificial Sequence Synthetic polynucleotide
  misc_feature (21)..(21): n is a, c, g, or t
gtgcccgtctgctggggcaangg

SEQ ID NO: 417 - DNA, 23 bp, Artificial Sequence
  Description of Artificial Sequence Synthetic polynucleotide
  misc_feature (21)..(21): n is a, c, g, or t
aacctcgtgcccgtctgctgngg

SEQ ID NO: 418 - DNA, 23 bp, Artificial Sequence
  Description of Artificial Sequence Synthetic polynucleotide
  misc_feature (21)..(21): n is a, c, g, or t
ggcggctggggtgtagaagcngg

SEQ ID NO: 419 - DNA, 23 bp, Artificial Sequence
  Description of Artificial Sequence Synthetic polynucleotide
  misc_feature (21)..(21): n is a, c, g, or t
agtcacattctctatggtcangg

SEQ ID NO: 420 - DNA, 23 bp, Artificial Sequence
  Description of Artificial Sequence Synthetic polynucleotide
  misc_feature (21)..(21): n is a, c, g, or t
ctggtttgatgaccaacttcngg

SEQ ID NO: 421 - DNA, 23 bp, Artificial Sequence
  Description of Artificial Sequence Synthetic polynucleotide
  misc_feature (21)..(21): n is a, c, g, or t
cattcattatgcctgggattngg

SEQ ID NO: 422 - DNA, 23 bp, Artificial Sequence
  Description of Artificial Sequence Synthetic polynucleotide
  misc_feature (21)..(21): n is a, c, g, or t
tgctagagtcacattctctangg

SEQ ID NO: 423 - DNA, 23 bp, Artificial Sequence
  Description of Artificial Sequence Synthetic polynucleotide
  misc_feature (21)..(21): n is a, c, g, or t
gggcacgaggttccctggggngg

SEQ ID NO: 424 - DNA, 23 bp, Artificial Sequence
  Description of Artificial Sequence Synthetic polynucleotide
  misc_feature (21)..(21): n is a, c, g, or t
ggctcctttgccccagcagangg

SEQ ID NO: 425 - DNA, 23 bp, Artificial Sequence
  Description of Artificial Sequence Synthetic polynucleotide
  misc_feature (21)..(21): n is a, c, g, or t
attattggacatccagatacngg

SEQ ID NO: 426 - DNA, 23 bp, Artificial Sequence
  Description of Artificial Sequence Synthetic polynucleotide
  misc_feature (21)..(21): n is a, c, g, or t
tttcatcattcattatgcctngg

SEQ ID NO: 427 - DNA, 23 bp, Artificial Sequence
  Description of Artificial Sequence Synthetic polynucleotide
  misc_feature (21)..(21): n is a, c, g, or t
ttctacaccccagccgccccngg

SEQ ID NO: 428 - DNA, 23 bp, Artificial Sequence
  Description of Artificial Sequence Synthetic polynucleotide
  misc_feature (21)..(21): n is a, c, g, or t
tcagggacacatctcctttgngg

SEQ ID NO: 429 - DNA, 23 bp, Artificial Sequence
  Description of Artificial Sequence Synthetic polynucleotide
  misc_feature (21)..(21): n is a, c, g, or t
gctcctttgccccagcagacngg

SEQ ID NO: 430 - DNA, 23 bp, Artificial Sequence
  Description of Artificial Sequence Synthetic polynucleotide
  misc_feature (21)..(21): n is a, c, g, or t
ctcagaagtggaatacagagngg

SEQ ID NO: 431 - DNA, 23 bp, Artificial Sequence
  Description of Artificial Sequence Synthetic polynucleotide
  misc_feature (21)..(21): n is a, c, g, or t
cgaggttccctggggcggctngg

SEQ ID NO: 432 - DNA, 23 bp, Artificial Sequence
  Description of Artificial Sequence Synthetic polynucleotide
  misc_feature (21)..(21): n is a, c, g, or t
gccacattcaaacacaggacngg

SEQ ID NO: 433 - DNA, 23 bp, Artificial Sequence
  Description of Artificial Sequence Synthetic polynucleotide
  misc_feature (21)..(21): n is a, c, g, or t
ggtgctcaggactgatgaaangg

SEQ ID NO: 434 - DNA, 23 bp, Artificial Sequence
  Description of Artificial Sequence Synthetic polynucleotide
  misc_feature (21)..(21): n is a, c, g, or t
aggtcacccctgcaccgactngg

SEQ ID NO: 435 - DNA, 23 bp, Artificial Sequence
  Description of Artificial Sequence Synthetic polynucleotide
  misc_feature (21)..(21): n is a, c, g, or t
ctctctgccgagtcggtgcangg

SEQ ID NO: 436 - DNA, 23 bp, Artificial Sequence
  Description of Artificial Sequence Synthetic polynucleotide
  misc_feature (21)..(21): n is a, c, g, or t
tctctctgccgagtcggtgcngg

SEQ ID NO: 437 - DNA, 23 bp, Artificial Sequence
  Description of Artificial Sequence Synthetic polynucleotide
  misc_feature (21)..(21): n is a, c, g, or t
ccaaggatgcttaccaccagngg

SEQ ID NO: 438 - DNA, 23 bp, Artificial Sequence
  Description of Artificial Sequence Synthetic polynucleotide
  misc_feature (21)..(21): n is a, c, g, or t
tctctgccgagtcggtgcagngg

SEQ ID NO: 439 - DNA, 23 bp, Artificial Sequence
  Description of Artificial Sequence Synthetic polynucleotide
  misc_feature (21)..(21): n is a, c, g, or t
cccctggtggtaagcatcctngg

SEQ ID NO: 440 - DNA, 23 bp, Artificial Sequence
  Description of Artificial Sequence Synthetic polynucleotide
  misc_feature (21)..(21): n is a, c, g, or t
tccaaggatgcttaccaccangg

SEQ ID NO: 441 - DNA, 23 bp, Artificial Sequence
  Description of Artificial Sequence Synthetic polynucleotide
  misc_feature (21)..(21): n is a, c, g, or t
ggtggtaagcatccttggaangg

SEQ ID NO: 442 - DNA, 23 bp, Artificial Sequence
  Description of Artificial Sequence Synthetic polynucleotide
  misc_feature (21)..(21): n is a, c, g, or t
gtgaagtctctctgccgagtngg

SEQ ID NO: 443 - DNA, 23 bp, Artificial Sequence
  Description of Artificial Sequence Synthetic polynucleotide
  misc_feature (21)..(21): n is a, c, g, or t
atgcttaccaccaggggacangg

SEQ ID NO: 444 - DNA, 23 bp, Artificial Sequence
  Description of Artificial Sequence Synthetic polynucleotide
  misc_feature (21)..(21): n is a, c, g, or t
ttccaaggatgcttaccaccngg

SEQ ID NO: 445 - DNA, 23 bp, Artificial Sequence
  Description of Artificial Sequence Synthetic polynucleotide
  misc_feature (21)..(21): n is a, c, g, or t
agtcggtgcaggggtgacctngg

SEQ ID NO: 446 - DNA, 23 bp, Artificial Sequence
  Description of Artificial Sequence Synthetic polynucleotide
  misc_feature (21)..(21): n is a, c, g, or t
acttcactgcagcctttccangg

SEQ ID NO: 447 - protein, 30 aa, Artificial Sequence
  Description of Artificial Sequence Synthetic polypeptide
  MISC_FEATURE: This sequence may encompass 2-6 “Gly Gly Gly Gly Ser” repeating units
GGGGSGGGGSGGGGSGGGGSGGGGSGGGGS

SEQ ID NO: 448 - protein, 50 aa, Artificial Sequence
  Description of Artificial Sequence Synthetic polypeptide
  MISC_FEATURE: This sequence may encompass 1-3 “Gly Gly Gly Gly Ser” repeating units, 1 “Gly Gly Gly Gly Pro” repeating unit, and 1 “Gly Gly Gly Gly Cys” repeating unit.
  MISC_FEATURE: This sequence may encompass 1-3 “Gly Gly Gly Gly Ser” repeating units, 1 “Gly Gly Gly Gly Pro” unit, and 1 “Gly Gly Gly Gly Cys” unit.
GGGGSGGGGCGGGGSGGGGSGGGGPGGGGSGGGGSGGGGSGGGGCGGGGS

SEQ ID NO: 449 - protein, 20 aa, Artificial Sequence
  Description of Artificial Sequence Synthetic polypeptide
  MISC_FEATURE: This sequence may encompass 3-4 “Gly Gly Gly Gly Ser” repeating units
GGGGSGGGGSGGGGSGGGGS

SEQ ID NO: 450 - protein, 20 aa, Artificial Sequence
  Description of Artificial Sequence Synthetic polypeptide
GGGGSGGGGSGGGGSGGGGS

SEQ ID NO: 451 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS